(19) United States
(12) Patent Application Publication
(10) Pub. No.: US 2004/0116448 A1
(43) Pub. Date: Jun. 17, 2004

(54) Substances for the therapy of diseases caused by highly proliferating cells

(76) Inventors: Hans-Gunther Schmalz, Koln (DE); Aram Prokop, Berlin (DE); Thomas P. Wieder, Dusslingen (DE); Peter T. Daniel, Berlin (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/474,318

(22) PCT Filed: Apr. 6, 2002
(86) PCT No.: PCT/EP02/03835

(30) Foreign Application Priority Data
Apr. 7, 2001 (DE) 10117479.9
May 29, 2001 (EP) 0111305.1

(51) Int. Cl. A61K 31/52; A61K 31/513

(52) U.S. Cl. 514/263.24; 514/269

(57) ABSTRACT

The invention relates to medicaments and compounds for the treatment of diseases caused by highly proliferating cells, such as tumor cells, especially blasts of children suffering from acute leukemia. The invention also relates to the preparation of the corresponding medicaments and compounds.

The present invention relates to substances and medicaments for the treatment of diseases caused by highly proliferating cells, and to methods for the preparation of such substances and medicaments.

The production of tumors or other, benign, hyperproliferative diseases, such as psoriasis or keloid, can be attributed to a disturbed balance between tissue regeneration and the regulated death of cells from the tissue. In clinical practice, cytotoxic treatment methods, such as chemotherapy, radiation therapy and hyperthermia, are used to attempt to restore this disturbed balance and to kill the excess tumor cells at the same time. It is generally recognized that most chemotherapeutic agents currently employed in clinical practice display their activity by inducing apoptosis (programmed cell death) (Hannun, 1997). However, part of the patients afflicted with malignant tumors early develop a resistance against chemotherapy and radiation therapy, or they are primarily refractory towards therapy (Hickman, 1996). It is further known that the primary tumor and the metastases often respond quite differently to cytotoxic therapies. From more recent studies, it is probable that the cause of resistances resides in different defects in the apoptosis signal cascade (Raisova et al., 2000).

Especially the therapy of recurrence of childhood acute lymphoblastic leukemia (ALL), the most frequent malignant disease in childhood, still fails to be satisfactorily solved. Thus, despite of aggressive therapies, about 70% of the afflicted children will die in recurrent ALL. The situation is similar with other tumor diseases, which are partly difficult to cure, such as thyroid carcinoma, the very frequent mamma carcinoma, medulloblastoma, which is difficult to deal with, or gliomas. A benign skin disease which is also treated with such therapies is psoriasis. It is one of the most frequent diseases of the skin from which two to four percent of humans are suffering. The therapy of this disease is also highly in need of improvement.

Thus, there is a strongly felt need to develop substances and medicaments with which the percentage of successful healings and survival chances of patients suffering from the above stated diseases are improved. Especially for tumor diseases and leukemias, it is important to provide new medicaments which act against unnaturally proliferating cells in a highly selective way and attack healthy cells as little as possible. In addition, it is of particular importance to provide therapeutic agents against those tumors which prove resistant towards previously known substances.

Surprisingly, this object is achieved by medicaments of the following structural formulas (1a or 1b):

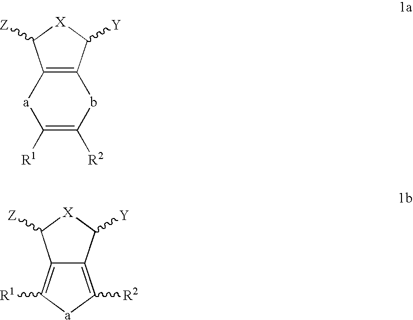

wherein

X=O, S, CH₂, NR, no bond, with the proviso that, if X=O, then the compound of formula 1a is associated with a metal-ligand complex according to formula 1′a;

Y=adenine, cytosine, guanine, uracil, thymine, bromouracil, purines or pyrimidines and their derivatives, nucleobases, heterocycles, aminoalkyl, aminoaryl or other residues which are capable of hydrogen bonding;

Z=alkyl, fluoroalkyl, aryl, fluoroaryl, —(CH₂)ₙOR′, with R′=H, SiR₃, alkyl, fluoroalkyl, aryl, fluoroaryl, acyl, and n=0 to 5, wherein preferably n=1;

a=no bond, a single bond or CH₂, in formula 1b also CH₂CH₂;

b=no bond, a single bond or CH₂; a and b being selected in such a way that the moiety comprising a and b includes a 1,3-diene;

R¹=—H, F, alkyl, fluoroalkyl, aryl, fluoroaryl, OR, —CO₂R, —SO₂OR, —CONR₂;

R²=—H, F, alkyl, fluoroalkyl, aryl, fluoroaryl, OR, —CO₂R, —CONR₂, —SO₂OR;

R=H, branched and linear alkyls, especially methyl, ethyl, propyl, thexyl, tert-butyl;

and/or salts thereof.

When a, b or X is stated as “no bond”, this means that the respective two carbon atoms which are linked to a, b or X in the structural formula, are not interlinked. However, since the moiety comprising a and b is supposed to include a 1,3-diene, it is not possible that both a and b are “no bond”.

The moiety comprising a and b may be present, for example, as a cyclobutadiene ring, cyclopentadiene ring or as an open 1,3-butadiene; in the latter case, either R¹ or R² may be linked with the terminal carbon atom.

In the SiR₃ group, R are preferably methyl, ethyl, propyl, tert-butyl or thexyl residues.

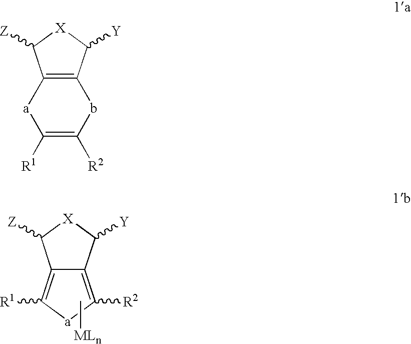

Further suitable are medicaments of structural formulas 1′a and 1′b in which the 1,3-diene is bonded to a metal fragment MLₙ, wherein:

n is an integer of from 2 to 4;

M=Mn, Fe, Co, Ru; and

L=CO, CN—R, CN, CR₂, COR, Hal, cyclopentadienyl (Cp) or a substituted Cp derivative, and R is selected as stated for structural formulae 1a and 1b;

and/or salts thereof.

The same or different Ls may be bonded to one M.

The metal fragment is preferably an Fe(CO)₃ complex.

Particularly preferred are medicaments having a structural formula 2, 3, 4a, 4b or 4c:

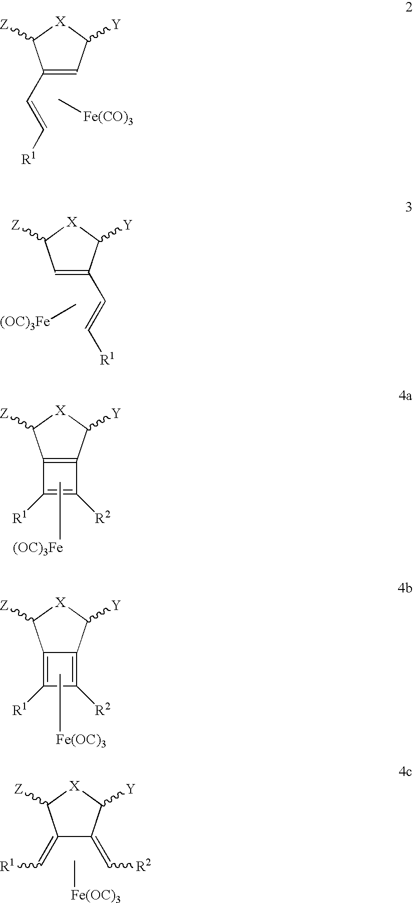

wherein the Fe(CO)₃ unit is η⁴-bonded;

X=O, CH₂ or no bond; and

Y, Z, R¹ and R² are selected as for structural formulae 1a and 1b.

The structural formulae 4a and 4b are resonance formulae 4c can be obtained from 4b by a formal ring opening.

Particularly suitable are medicaments of structural formula 5:

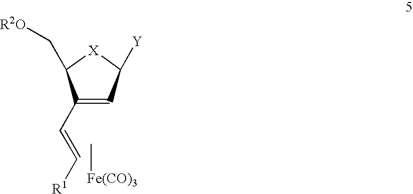

wherein

X=O, CH₂;

Y=adenine, cytosine, guanine, uracil, thymine, bromouracil, purines or pyrimidines and their derivatives, nucleobases, heterocycles;

R¹=—H, —CO₂R; and

R²=H, SiR₃, alkyl, fluoroalkyl,aryl, fluoroaryl, acyl.

The invention also relates to substances, and methods for their preparation, of structural formula 5 in which:

R²=SiR₃, X=O;

Y and R¹ are selected as stated for structural formula 5, and R is selected as stated for structural formulae 1a and 1b. However, these compounds and methods for their preparation are not included in the subject matter of the invention if Y is bromouracil, uracil or methyluracil and at the same time R² is thexyl-(CH₃)₂Si. In these cases, the invention relates to the corresponding medicaments.

In particular, the invention relates to the compounds of the following structural formulae:

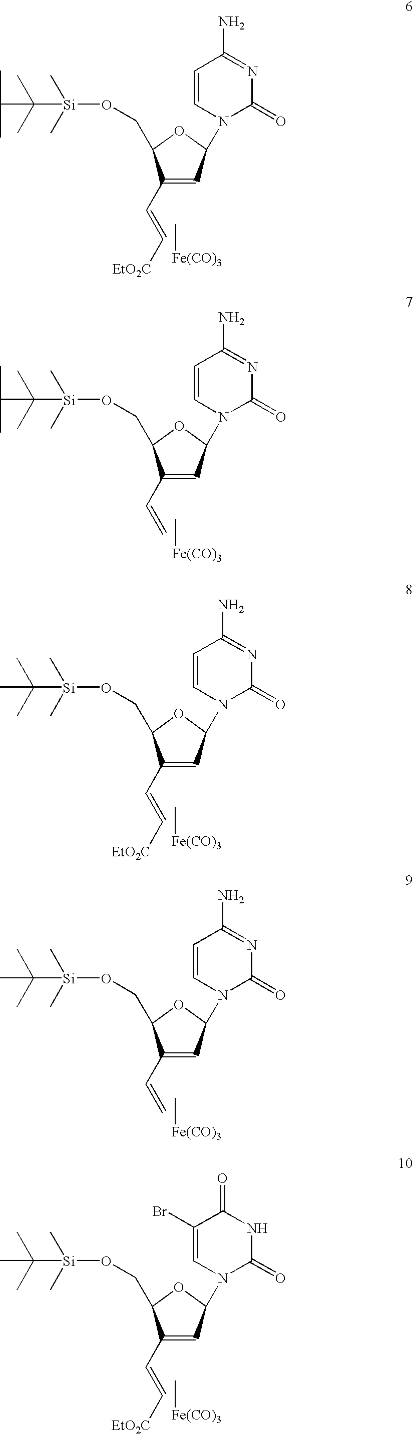

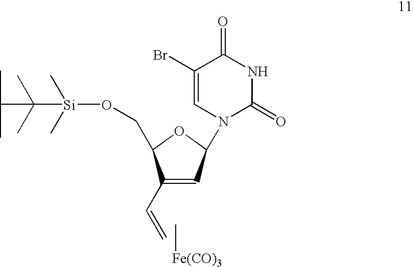

The invention also relates to a compound of structural formula 35:

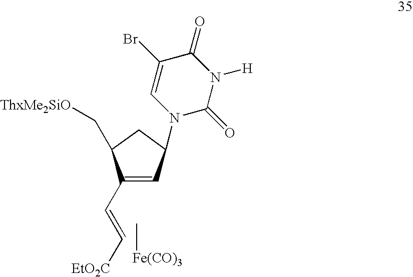

The invention also relates to the racemates and enantiomers of the compounds according to the invention.

According to the invention, the compounds according to the invention and the compounds of structural formula 1a

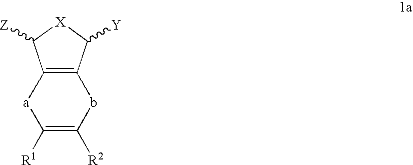

wherein X=O, S, CH₂, NR or no bond; and

Y, Z, a, b, R¹ and R² are selected as in claim 1;

are used for preparing a medicament for the treatment of malignant diseases of the bone marrow or other hematopoetic organs' solid tumors, epithelial tumors, benign or semimalignant fast-proliferating tumors or skin diseases, especially psoriasis vulgaris, keloids and basaliomas, lymphomas, especially Hodgkin's and non-Hodgkin lymphomas, inflammatory, chronic inflammatory, bacterial and auto-immune diseases, and for antibacterial, antimycotic, antiprotozoan, antiplasmodium, antihelminthic or immunosuppressant therapies.

The medicaments and compounds according to the invention are unexpectedly suitable for the therapy of pathologically fast proliferating tissues, especially bone marrow, but also solid tumors, such as epithelial tumors or, in particular, brain tumors. Further, the applicability of the substances described also extends to the treatment of benign hyperproliferative diseases of the skin, such as psoriasis or keloid. The medicaments and substances of the invention are characterized by being particularly suitable for selectively inhibiting the growth of highly proliferating cells. They thereby induce the apoptosis of highly proliferating cells and thus cause their destruction, healthy cells being affected very little.

The substances are particularly capable of penetrating membranes, which results in a high intracellular concentration of the active substance. Therefore, the high effectiveness is probably achieved by the pronounced lipophilicity of the substances. The substances are basically different from previously known nucleoside analogues used for therapy, such as cytarabine and fludarabine-5′ dihydrogen-phosphate. They are capable of breaking existing resistances towards cytostatic agents.

The medicaments and compounds of the invention are especially suitable for the treatment of tumor diseases and leukemia. They induce apoptotic cell death not only in permanent cell lines produced from tumor cells (BJAB cells), but also in primary cells of patients suffering from acute lymphoblastic leukemia (ALL). Thus, the substances according to the invention can be employed against tumor diseases of the bone marrow, but also against tumors of different origin, such as epithelial tumors, sarcomas or malignant diseases of the skin etc. In particular, it may be noted that trespassing the blood-brain barrier is possible with the developed substances due to their lipophilicity, and therefore they may also be employed for malignant brain tumors, such as medulloblastoma or gliomas. All in all, the substances according to the invention can be used for the treatment of malignant diseases of the bone marrow or other hematopoetic organs, solid tumors, epithelial tumors, benign or semimalignant fast-proliferating skin diseases, especially psoriasis vulgaris, keloids and basaliomas, as well as inflammatory and chronic inflammatory diseases. They are also suitable for antiviral, antibacterial, antimycotic, antiprotozoan, antihelminthic or immunosuppressant therapies.

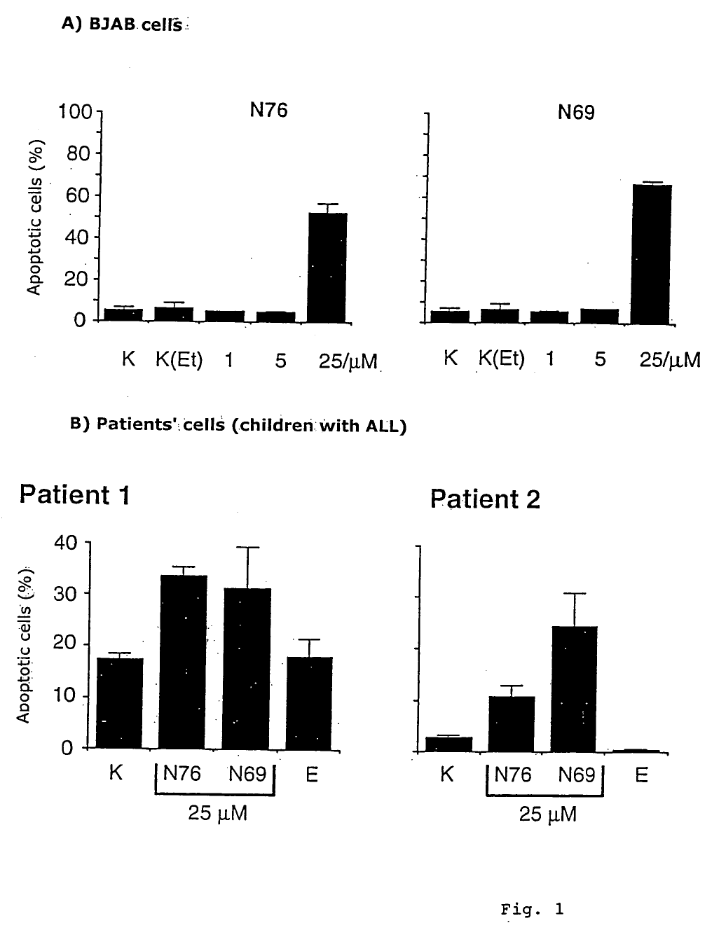

FIG. 1A shows that the substances N76 (compound of structural formula 5 with R¹=CO₂Et, R²=thexyldimethylsilyl, X=O, Y=bromouracil) and N69 (compound of structural formula 6) induce apoptosis in 60-70% of BJAB cells in a concentration of 25 μmol/l in the culture medium. The measurement of apoptosis is based on a method which detects the fragmentation of DNA on the level of individual cells, which is typical of apoptosis and distinguishes this form of cell death from necrosis. Thus, BJAB cells were treated with different concentrations of N76 or N69 for 24 h. Controls contained corresponding amounts of the solubilizer ethanol. After the treatment, the fragmentation of DNA was measured by staining with propidium iodide followed by quantification by flow cytometry as described by Essmann et al. (2000). The values are stated as % of apoptotic cells based on the total population±SD (n=3).

However, the substances exhibit pro-apoptotic activity not only on permanent cell lines, but also on lymphoblasts obtained directly from children suffering from ALL (FIG. 1B). In contrast to the previously known and very widely employed chemotherapeutic agent epirubicin (abbreviated by E), the substances N76 and N69 induce apoptosis in vitro also in therapy-resistant cells of patients (FIG. 1B). In this experiment, lymphoblasts from children afflicted with acute lymphoblastic leukemia (ALL) were isolated and, after dilution with cell culture medium, treated for 36 h with 25 μM N76, 25 μM N69 or with 9 μM epirubicin (E) as a positive control. Controls contained corresponding amounts of the solubilizer ethanol. After the treatment, the fragmentation of the DNA was measured by staining with propidium iodide followed by quantification by flow cytometry as described by Essmann et al. (2000). The values are stated as % of apoptotic cells based on the total population±SD (n=3).

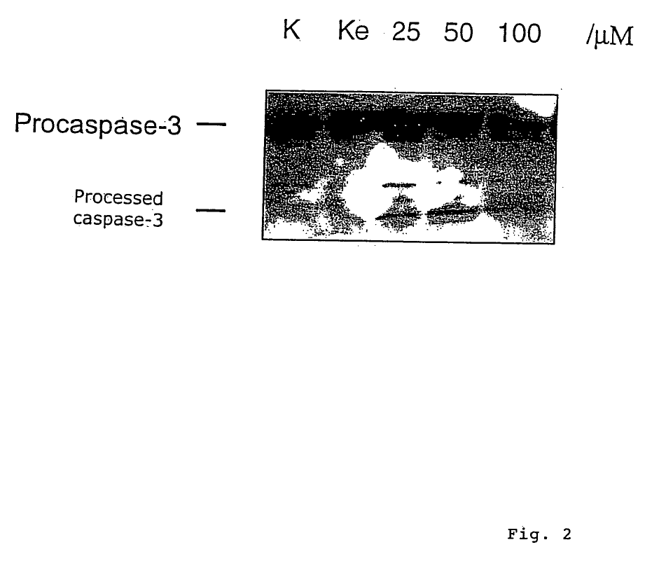

In the course of apoptosis, a family of cysteine proteases, the so-called caspases, which lyse the cells from inside in the course of the death program (Cohen, 1997), are activated. To further examine the specificity of the substances according to the invention, the processing and activation of caspase-3 was demonstrated in a Western blot (FIG. 2). Thus, BJAB cells were treated for 24 h with different concentrations of N76. Controls contained corresponding amounts of the solubilizer ethanol (Ke). After the treatment, the processing of procaspase-3 was determined by specific immunodetection in a Western blot as described by Essmann et al. (2000). The positions of procaspase-3 and the processed subunit in the SDS polyacrylamide gel are marked by horizontal bars on the left margin of FIG. 2. The addition of 25 or 50 μmol/l of substance N76 to the medium of BJAB cells induces the processing of procaspase-3 within these cells. The specific immunochemical detection of the active subunit of caspase-3 in treated cells in contrast to the corresponding control cells can be seen. The result shows clearly that the substances according to the invention specifically induce an apoptotic cascade.

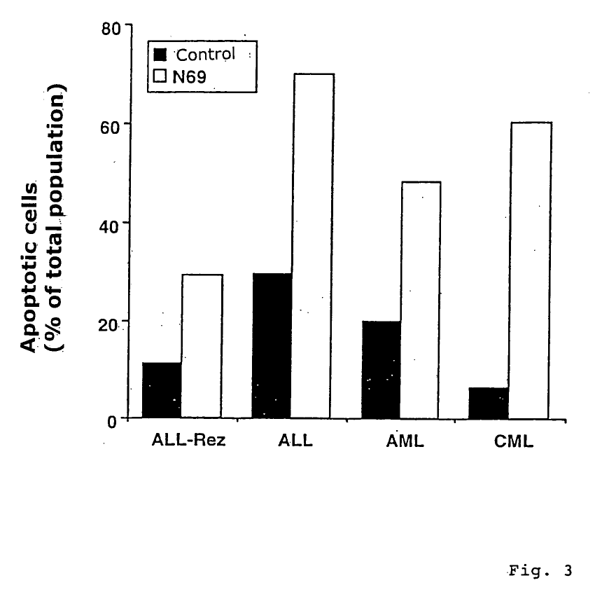

The usefulness of substances according to the invention for the therapy of various malignant diseases of the hematopoetic system was tested on cells from patients suffering from different diseases. Thus, cells from patients suffering from different leukemias were isolated and, after dilution with cell culture medium, treated for 36 h with 25 μM N69. Controls contained corresponding amounts of the solubilizer ethanol. After the treatment, the fragmentation of the DNA was measured by staining with propidium iodide followed by quantification by flow cytometry as described by Essmann et al. (2000). The result can be seen in FIG. 3. The measured values are stated as % of apoptotic cells based on the total population and represent the mean value from duplicate experiments. FIG. 3 shows that 25 μmol/l N69 induces apoptotic cell death both in cells from patients with a recurrence of acute lymphoblastic leukemia .(ALL-Rez) and in cells from patients with a first manifestation of this disease (ALL), in cells from patients suffering from acute myeloblastic leukemia (AML) and in cells from patients suffering from chronic myeloblastic leukemia, (CML).

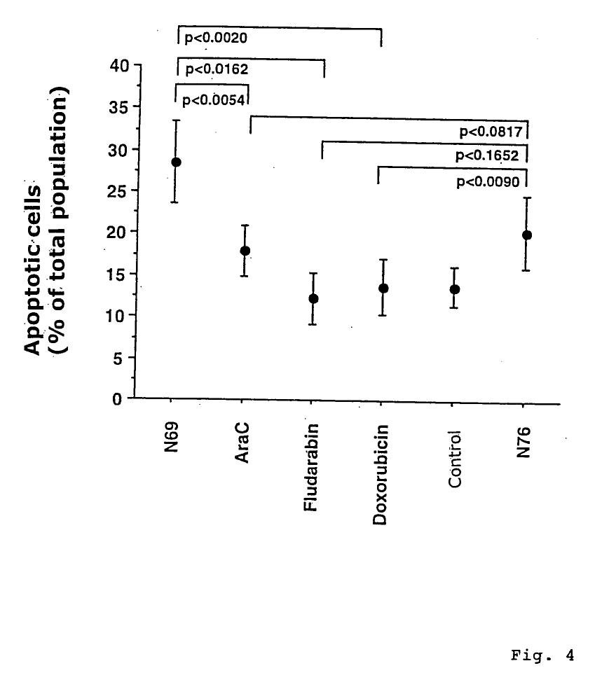

Thus, with N76 and N69, two substances of general structural formula 2 are provided as effective agents against certain tumor cells, especially those of childhood ALL, but also against other malignant diseases of different origin. In summary, FIG. 4 shows that the substance N69, in particular, exhibits a significantly better pro-apoptotic effect in vitro towards primary ALL cells as compared to the established cytostatic agents doxorubicin, cytarabin and fludarabin-5′ dihydrogenphosphate. In these experiments, the cells from eleven ALL patients were isolated and, after dilution with cell culture medium, treated for 36 h with 25 μM N69, 25 μM N76 or corresponding amounts of cytarabin (AraC), fludarabin or doxorubicin. Controls contained corresponding amounts of the solubilizer ethanol. After the treatment, the fragmentation of the DNA was measured by staining with propidium iodide followed by quantification by flow cytometry as described by Essmann et al. (2000). P values were calculated from a paired t test and are stated in FIG. 4 directly for the individual pairs to be compared. P values of <0.05 are considered statistically significant.

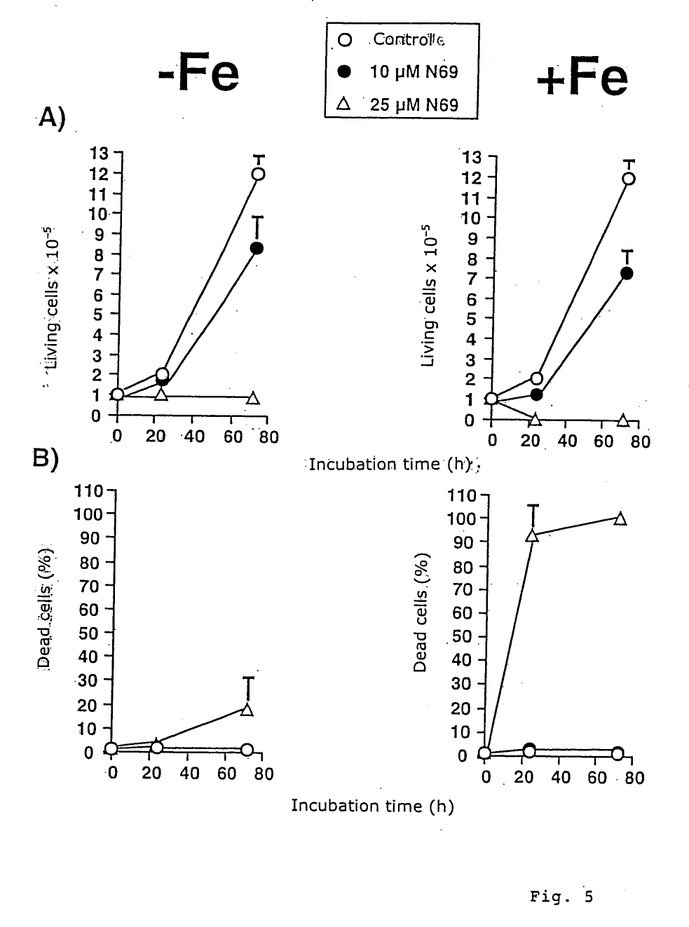

To further characterize the role of the various components of the newly described substances, the iron-complexed compound N69 and the compound obtainable from N69 by cleaving the Fe(CO)₃ group were prepared and employed in a combined proliferation and cell death assay. Thus, BIAB cells were treated for 24 h and 72 h. with different concentrations of N69 or the corresponding compound lacking the iron complex. Controls contained corresponding amounts of the solubilizer ethanol. After the treatment, the number of cells was counted in a Neubauer counting chamber, and the number of dead cells was counted by trypan blue staining as described by Wieder et al. (1995). FIG. 5A shows the total number of living cells times 10⁻⁵±SD (n=3) after incubation with control medium (open circles), 10 μM N69 (solid circles) or 25 μM N69 (open triangles) after 24 h and 72 h. FIG. 5B states the proportion of dead cells in %±SD (n=3) as determined at these times at the respective concentration. The left column shows the result after treatment with the decomplexed substance, and the right column shows the result for the complexed N69. As can be easily seen, the complexed iron plays an important role in the cell-death inducing activity of the substances. The complexed iron shifts the dose-effect curve of the cell-death inducing effect of N69 towards lower concentrations. While 25 μM of the iron-free compound induces cell death in only about 20% of the cells after 72 h, about 90% of the treated cells have died in the presence of 25 μM of iron-containing N69 already after 24 h (FIG. 5B). In contrast, for the antiproliferative properties of N69, the nucleoside fraction is rather the responsible one since both tested substances significantly reduce proliferation at 10 μM and completely block it at 25 μM (see FIG. 5A). That means, the present specification describes new classes of substances which possess two structural features which are essential to activity. However, it may be pointed out that those substances of the invention which are not applied as iron complexes also exhibit a high therapeutic effectiveness.

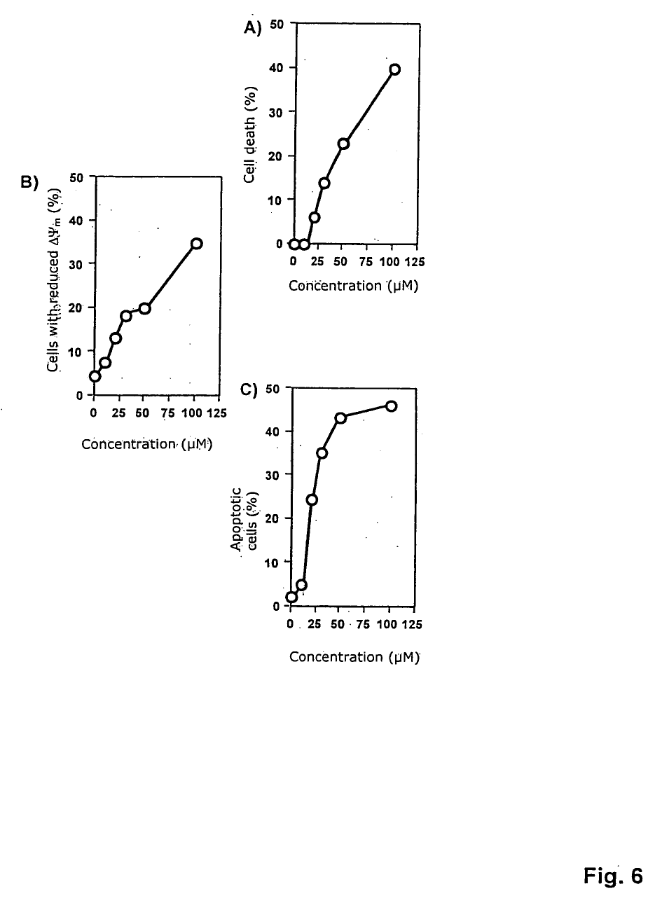

In a further embodiment, a pro-apoptotic activity is demonstrated for the newly synthesized carbocyclic nucleoside analogue JV-206-1 (structural formula). FIG. 6A shows that incubation of BJAB cells with JV-206-1 (35) for 48 hours induces cell death. Further studies had the result that the BJAB cells are driven to the mitochondrial apoptosis signal pathway by JV-206-1. This was demonstrated by staining the cells with the mitochondrion-specific stain JC-1 as described by Wieder et al. Thus, the incubation of the cells with JV-206-1 resulted in a concentration-dependent increase of the fraction of cells having a reduced mitochondrial membrane potential (ΔΨₘ), which indicates a strong activation of the mitochondria during the apoptotic process (FIG. 6B). In addition, the fragmentation of the DNA was measured as a specific characteristic of apoptosis as described by Essmann et al. (2000). It was established that the major part of the cells die at JV-206-1 concentrations of 20 μM by apoptosis (FIG. 6C). In these experiments, the proportion of apoptotic cells was between 20% and 48% of the total population, depending on the concentration and incubation time.

The medicaments according to the invention can be applied topically or intravenously. In an intravenous application, the substances are administered in a concentration range of between 0.1 and 100 μg/ml, based on the blood volume of the patient. The substances are rubbed into the afflicted skin in a concentration of from 0.1 to 5% by weight, based on the final preparation.

The present invention also relates to methods for the synthesis of the compounds according to the invention, which are summarized in schemes 1 to 5. In one embodiment of the invention, the method comprises the following steps:

Step (a): conversion of a glycoside (preferably a glucose derivative) 12 into a protected compound 13 by reaction with a trialkylsilyl reagent;

step (b): conversion of 13 into one or more epoxides of type 14 under Mitsunobu conditions (azodicarboxylic acid diester/trialkylphosphane);

step (c): ringcontraction of 14 (also as a mixture) to form aldehydes of the type 15 by heating with LiBr in toluene in the presence of donor cosolvents, such as tetraalkylureas;

step (d): olefination of aldehyde 15 to form dienes of the type 16, preferably by a Wittig reaction;

step (e): complexing of 16 to form 17 using Fe₂(CO)₉ or other reagents capable of transferring an Fe(CO)₃ group;

step (f): introduction of a nucleobase or a group Y (according to formula 5) by a diastereoselective iron-supported nucleophilic substitution, preferably using silylated nucleobases in the presence of a Lewis acid (Hessler 1994).

The general synthetic route (scheme 1) was developed and published within the scope of the dissertation of Erik Hessler (Universität Frankfurt am Main 1993) and the Diplomarbeit of Andre Majdalani (Universität Frankfurt am Main 1994). Steps (a), (b) and (c) (preparation of the aldehyde intermediates of type 15 with R₃Si=thexyldimethylsilyl) are based on a method known from the literature (Rehnberg 1990). The separation of diastereomeric by-products is effected by chromatography and/or crystallization on the stage of 17, 18 or 19.

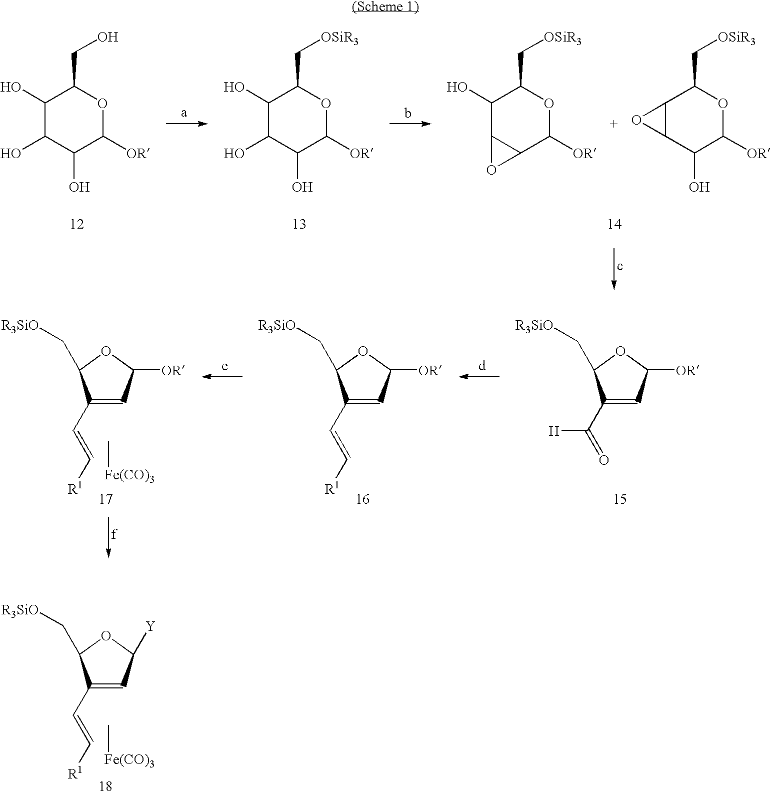

For varying the group R² in the preparation of compounds of type 5 (Scheme 2):

(g) the silyl group of 17 is cleaved off with fluoride;

(h) the free OH function is again etherified or esterified; and

(f) the group Y is again introduced by diastereoselective iron-supported nucleophilic substitution, preferably using silylated nucleobases in the presence of a Lewis acid.

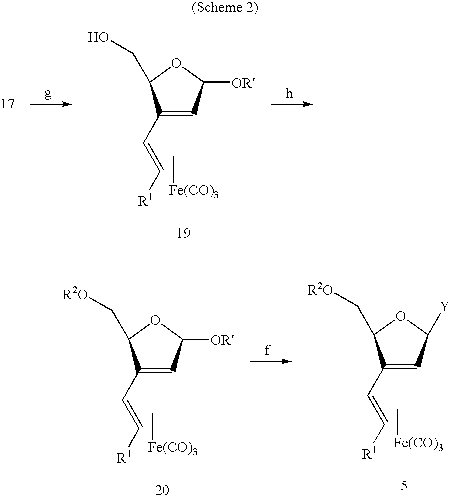

For example, step (f) may comprise the following reaction:

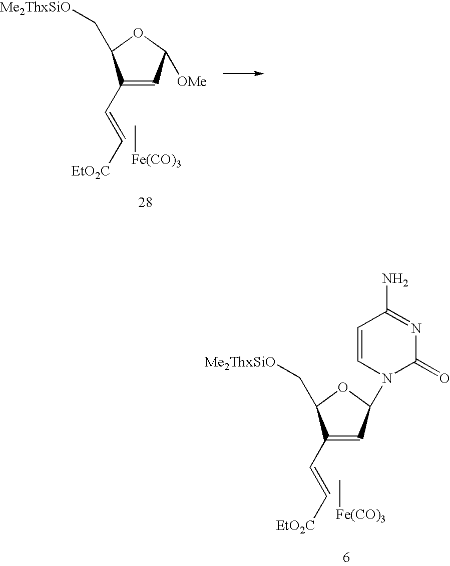

The present invention also relates to a method for the synthesis of substances of type 44 (type 2 with X=CH₂, e.g., compound 35) which are used as medicaments (Scheme 3). It comprises the following steps:

Steps (a, b, c): conversion of propargyl alcohol 36 into acetal 37 by C-silylation, oxidation of the alcohol to form the aldehyde, and acid-catalyzed acetalization with allyl alcohol;

step (d): cyclization of 37 by Pauson-Khand reaction or a Pauson-Khand-like reaction;

step (e): diastereoselective reduction of 38, preferably with sodium borohydride in the presence of cerium(III) chloride;

steps (f, g): desilylation and acetylation to form 40;

step (h): diastereoselective Pd-catalyzed introduction of a nucleobase or another (nucleophilic) group Y (according to formula 5);

steps (i, j): acid-catalyzed acetal hydrolysis to form a hydroxyaldehyde, followed by silylation, etherification or esterification of the OF function (variable introduction of R²);

step (k): olefination of aldehydes 42 to form dienes of type 43, preferably by a Wittig reaction (variable definition of R¹);

step (l): diastereoselective complexing of 43 to form 44 using Fe₂(CO)₉ or other reagents capable of transferring an Fe(CO)₃ group.

The general synthetic route up to the intermediates of type 42 (Scheme 3) has already been published (J. Velcicky, J. Lex, H.-G. Schmalz, Org. Lett. 2002, 4, 565-568). Steps (a) to (d) (preparation of the racemic Pauson-Khand product 38) were performed by analogy with a literature method (N. Jeong, B. Y. Lee, S. M. Lee, Y. K. Chung, S.-G. Lee, Tetrahedron Lett. 1993, 34, 4023).

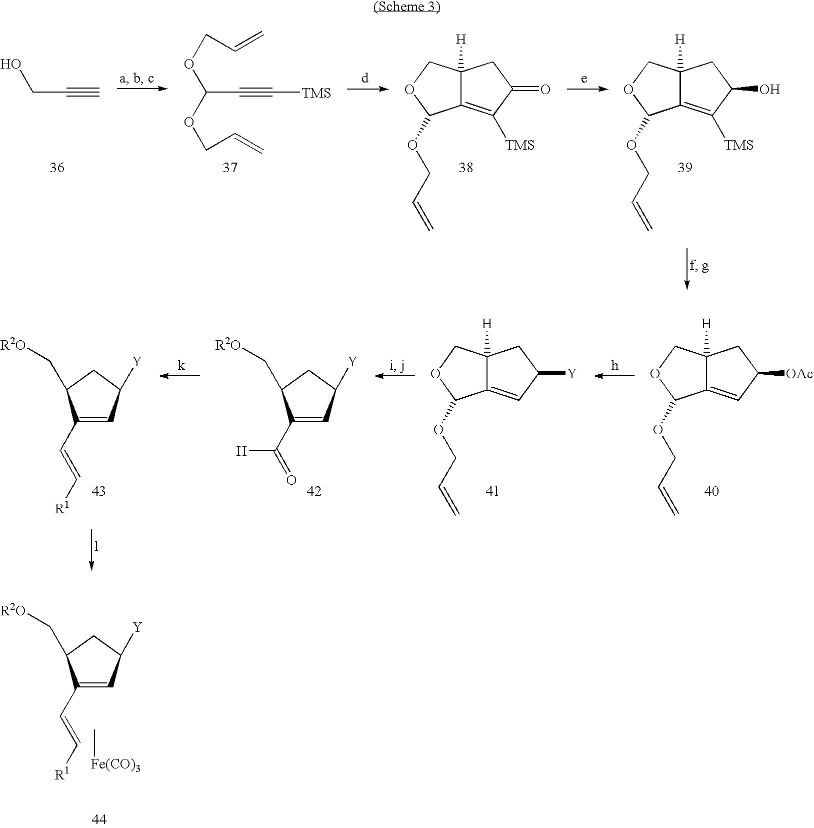

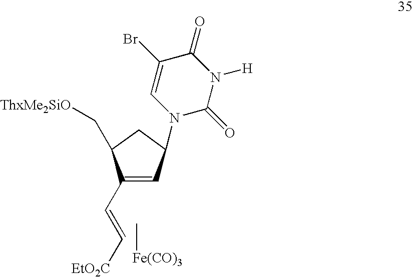

For checking the absolute configuration of the Pauson-Khand product 38, the chirogenic step (d) can be performed either enantioselectively (in the presence of chiral rhodium or iridium complexes), or the product can be resolved by kinetic optical resolution (e.g., as represented in Scheme 4 by oxazaborolidine-catalyzed borane reduction).

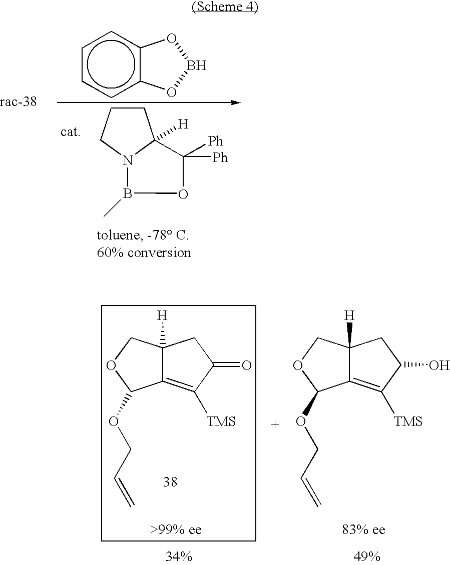

The present invention also relates to a method for the synthesis of substances of type 52 (type 3 with X=O, Z=CH₂OR², e.g., compound 53) which are used as medicaments (Scheme 5). It comprises the following steps:

Steps (a, b): conversion of ribonolactone 45 into the trityl-protected enol triflate 46 by tritylation of the primary OH function and reaction with trifluoromethanesulfonic acid anhydride in pyridine;

step (c): Pd-catalyzed coupling of 46 with a vinylstannane or vinylborane to form 47;

step (d): diastereoselective complexing of 47 to form 48 using Fe₂(CO)₉ or other reagents capable of transferring an Fe(CO)₃ group;

step (e): reduction of the lactone to form lactol 49 using diisobutylaluminum hydride;

step (f): acid-catalyzed trityl cleavage and parallel substitution of the OH group by an OMe group (methanol as solvent);

step (g): silylation, etherification or esterification of the OH function under basic conditions (variable introduction of R²);

step (h): introduction of a nucleobase or a group Y (according to formula 5) by a diastereoselective iron-supported nucleophilic substitution, preferably using silylated nucleobases in the presence of a Lewis acid.

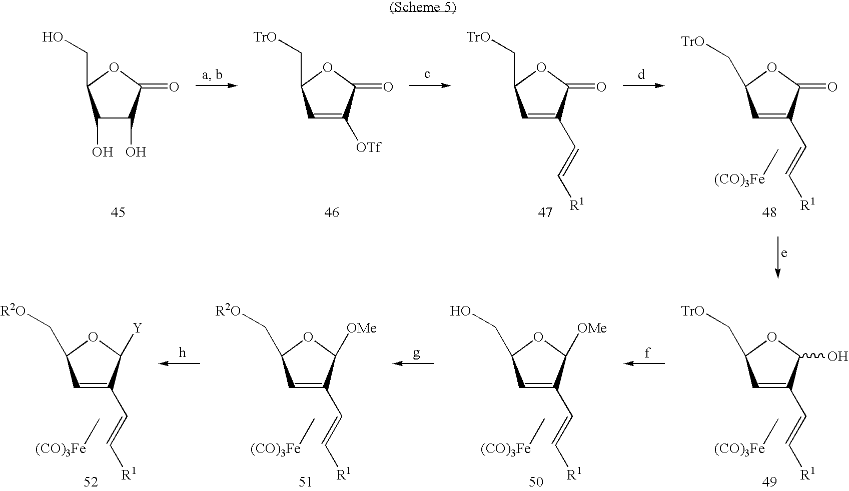

### Synthesis Protocols

1. Preparation of (+)-methyl-6-O-(dimethylthexylsilyl)-α-D-glucopyranoside (21)

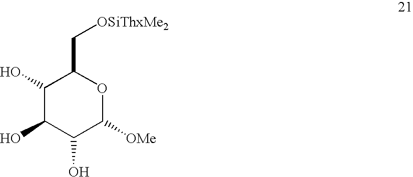

In a 100 ml Schlenk flask, 4.70 g (24.20 mmol) of (+)-methyl-α-D-glucopyranoside was dissolved in 30 ml of abs. pyridine in an argon atmosphere and subsequently cooled to 0° C. in an ice bath. With magnetic stirring and with an argon countercurrent, 5.0 ml (24.60 mmol) of thexyldimethylsilyl chloride was injected with a syringe, stirring was continued for 1 h at 0° C., and then the cooling bath was removed. After another 22.5 h at room temperature, two additions of 50 ml each of MeOH were made, followed by concentration under vacuum in a rotary evaporator. Subsequently, the reaction mixture was transferred into a separation funnel with 200 ml of ethyl acetate, the organic phase was washed twice with 100 ml each of a solution of 190 ml of H₂O, 20 ml of conc. HCl and 15 g of NH₄Cl, and with 100 ml each of saturated aqueous NaHCO₃ solution and saturated aqueous NaCl solution. The aqueous phases were extracted with 2×100 ml of ethyl acetate, and the combined organic phases were dried with Na₂SO₄. After filtration, the solution was concentrated under vacuum in a rotary evaporator, and the residue was purified by flash chromatography on 190 g of silica gel (ethyl acetate). After thorough drying in an oil-pump vacuum, 6.27 g (77%) of the analytically pure silyl ether 21 was obtained as a colorless solid. Melting point: 88-89° C. (ethyl acetate), colorless solid.—TLC: ethyl acetate, R_{f}=0.34.—FT-IR (KBr): 3375 (s and br., O—H), 2958, 2902, 2876 (s,s,s, sat. C—H), 1466, 1379, 1251, 1152, 1118, 1081 (m,m,m,s,s,s), 1052 (s, C—O), 831, 778 (s,m).—¹H NMR: (400 MHz, CD₃OD): δ=0.12 (ψs, 6H, (CH₃)₂Si), 0.87 (ψs, 6H, (CH₃)₂C), 0.91 (d, J=6.9 Hz, 6H, (CH₃)₂CH), 1.65 (sept, J=6.9 Hz, 1H, (CH₃)₂CH), 3.28 (ψt, J=8.9 Hz, 1H, 4-H), 3.35 (dd, ³J_{2,1}=3.7 Hz, ³J_{2,3}=9.7 Hz, 1H, 2-H); 3.39 (s, 3H, OMe), 3.51 (ddd, ³J_{5,6b}=2.1 Hz, ³J_{5,6a}=5.5 Hz, ³J_{5,4}=9.9 Hz, 1H, 5-H), 3.60 (ψt, J=9.5 Hz, 1H, 3-H),3.75 (dd, ³J_{6a,5}=5.5 Hz, ²J_{6a,6b}=11.2 Hz, 1H, 6-Ha), 3.89 (dd, ³J_{6b,5}=2.1 Hz, ²J_{6b,6a}=11.2 Hz, 1H, 6-Hb), 4.63 (d, J=3.7 Hz, 1H, 1-H).—Analysis: C₁₅H₃₂O₆Si (336.50) calc.: C: 53.54%, H: 9.58%; found: C: 53.28%, H: 9.88%.

2. Preparation of Epoxides of Types 22 and 23 as a Mixture

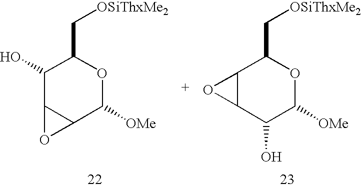

In a 500 ml three-necked flask equipped with a reflux condenser and Hg bubbler headpiece, 10.35 g (30.76 mmol) of the silyl ether 21 and 9.70 g (36.98 mmol) of triphenylphosphane were charged in 250 ml of abs. benzene under a protective gas and substantially dissolved by vigorous magnetic stirring. To this solution was added (under argon countercurrent) 5.3 ml (33.66 mmol) of DEAD with a syringe, and reaction was allowed to proceed for 1h at room temperature (the solution becoming turbid after 30 min) before it was heated to boil for 4 h. After cooling to room temperature, the reaction solution was transferred into a separation funnel and washed with 250 ml each of 1 N HCl solution and saturated aqueous NaCl solution. The aqueous phases were extracted with 2×300 ml and 1×150 ml of ether, and the combined organic phases were dried with Na₂SO₄. After filtration, the solution was concentrated under vacuum in a rotary evaporator, the residue was applied to silica gel with CH₂Cl₂ and subsequently purified (in two portions) by flash chromatography on 190 g of silica gel (n-hexane/ethyl acetate=2+1, then 5+3). After drying in an oil-pump vacuum, 8.71 g (89%) of a colorless solid was obtained which, as seen from ¹H NMR, (probably) was a mixture of four isomeric epoxides. The main product could be isolated by preparative HPLC (conditions see below) as a colorless solid in an analytically pure form.

Raw mixture:—TLC: n-hexane/ethyl acetate=1+1, R_{f}=0.34 and 0.41.—analyt. HPLC: n-hexane/ethyl acetate=10+6.67; MN Nucleosil 50-10; 2 ml/min; refractometry; retention times: 3.21 min, 3.43 min, 4.20 min, 5.13 min.—prep. HPLC: n-hexane/ethyl acetate=10+6.67; 0.1 L/min; RI 20.—Analysis: C₁₅H₃₀O₅Si (318.48); calc.: C: 56.57%, H: 9.49%; found: C: 56.80%, H: 9.39%.

Main product:—TLC: n-hexane/ethyl acetate=1+1, R_{f}=0.34.—analyt. HPLC: n-hexane/ethyl acetate=10+6.67; MN Nucleosil 50-10; 2 ml/min; refractometry; retention time: 4.20 min.—prep. HPLC: n-hexane/ethyl acetate=10+6.67; 0.1 l/min; RI 20.—FT-IR (KBr): \tilde{ν}=3441 (s and br., O—H), 2959, 2878 (s,m, sat. C—H), 1463, 1408 (m,m), 1257, 1149, 1130, 1105 (each s), 1059 (s, C—O), 1001, 983, 906, 822, 780, 763 (each s).—¹H NMR: (250 MHz, CDCl₃): δ=0.13 (ψs, 6H, (CH₃)₂Si), 0.85 (ψs, 6H, (CH₃)₂C), 0.88 (d, J=6.9 Hz, 6H, (CH₃)₂CH), 1.62 (sept, J=6.9 Hz, 1H, (CH₃)₂CH), 2.52 (broad s, 1H, D₂O exchangeable, 4-OH), 3.45 (s, 3H, OMe), 3.46 (dd, ³J_{3,4}=1.8 Hz, ³J_{3,2}=4.2 Hz, 1H, 3-H), 3.54 (dd, ³J_{2,1}=3.1 Hz, ³J_{3,2}=4.2 Hz, 1H, 2-H), 3.67 (ψdt, ³J_{5,6}=4.9 Hz, ³J=9.0 Hz, 1H, 5-H), 3.74-3.85 (m, inter alia, with J=5.3 Hz, J=10.5 Hz, 2H, 6-H), 3.93 (broad d, J=8.7 Hz, 1H, 4-H), 4.88 (dd, ⁴J_{1,5}=0.5 Hz, ³J_{1,2}=3.1 Hz, 1H, 1-H).—Analysis: C₁₅H₃₀O₅Si (318.48); calc.: C: 56.570%, H: 9.49%; found: C: 56.030/0, H: 9.30%.

3. Preparation of (−)-(2S,5S)-5-((dimethylthexylsilyloxy)methyl)-2-methoxy-2,5-dihydrofuran-4-carbaldehyde (24)

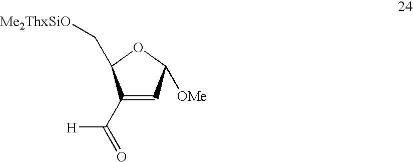

In a 500 ml three-necked flask equipped with a dropping funnel, water separator and reflux condenser (with Hg bubbler headpiece), 3.66 g (42.15 mmol) of LiBr and 5.10 ml (42.46 mmol) of tetramethylurea freshly distilled in a bulb tube (40-60° C., ca. 2 mbar) were charged in 200 ml of toluene in an argon atmosphere and heated to reflux in an oil bath with magnetic stirring. Subsequently, 3.14 g (9.86 mmol) of the above described mixture of epoxides (22, 23) dissolved in 150 ml of toluene was added dropwise to the boiling reaction solution within 2.5 h. The reaction was allowed to proceed for another 30 min, cooled to room temperature, and then 170 ml of ether was added. For separating off a brown polymerization product, the mixture was filtered through 50 g of silica gel, the residue washed with 150 ml of toluene/ether=2+1, and the solvent was removed under vacuum in a rotary evaporator. The brown oily residue was purified by flash chromatography on 220 g of silica gel (n-hexane/ethyl acetate=6+1). After drying in an oil-pump vacuum, 2.23 g (75%) of aldehyde 24 was obtained as an analytically pure red-brown oil.—TLC: n-hexane/ethyl acetate=4+1, R_{f}=0.37.—angle of rotation: [α]₅₈₉²⁰ =−68.7, (c=1.0 in CHCl₃);—CD: Θ (λ)=−19352 (233.0 nm), −2429 (335.0 nm), −2052 (341.0 nm), −2832 (348.0 nm), −1701 (357.0 nm), −2321 (365.0 nm), −679 (378.0 nm), −947 (384.0 nm), (c=0.007 in n-hexane).—UV (MeOH): λₘₐₓ(ε)=214.0 nm (8935).—FT-IR (film): \tilde{ν}=2958, 2868 (m,m, sat. C—H), 2830, 2717 (m,w, C—H-aldehyde), 1692 (s, C═O), 1465, 1354, 1252, 1190, 1136 (each m), 1042 (s, C—O), 969, 903, 833, 778 (each m).—¹H NMR: (270 MHz, CDCl₃): δ=0.02+0.06 (s+s, each 3H, (CH₃)₂Si), 0.78 (ψs, 6H, (CH₃)₂C), 0.82 (ψd, J=6.9 Hz, 6H, (CH₃)₂CH), 1.55 (sept, J=6.9 Hz, 1H, (CH₃)₂CH), 3.43 (s, 3H, OMe), 3.90 (ψd, J=2.5 Hz, 2H, CH₂O), 5.12-5.16 (m, 1H, 5-H), 5.91 (ψd, J=4.3 Hz, 1H, 2-H), 6.68 (ψs, 1H, 3-H), 9.88 (s, 1H, CHO).—Analysis: C₁₅H₂₈O₄Si (300.47); calc.: C: 59.96%, H: 9.39%; found: C: 59.99%, H: 9.34%.

4. Preparation of (−)-(2S,5S)-5-((dimethylthexylsilyloxy)methyl)-2-methoxy-4-vinyl-2,5-dihydrofuran (25)

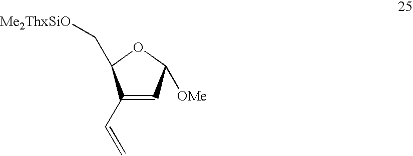

In a 250 ml Schlenk flask, 1.85 g (5.18 mmol) of methyltriphenylphosphonium bromide in 50 ml of abs. THF was charged in an argon atmosphere and cooled down to −40° C. With magnetic stirring and argon countercurrent, 3.0 ml (4.80 mmol) of n-butyllithium solution (1.6 M in hexane) was injected with a syringe, the reaction was allowed to proceed for 30 min (turning to yellow), cooled down to −78° C., and then 1.01 g (3.36 mmol) of aldehyde 24 dissolved in 20 ml of abs. THF was added to the reaction solution. The solution was allowed to slowly reach room temperature (ca. 4.5 h) while it turned orange, and transferred into a separation funnel with 200 ml of ether. After the addition of 200 ml of ice water, the phases were separated, and the organic phase was washed with 200 ml of saturated aqueous NaCl solution. The aqueous phases were extracted with 1×200 ml and 2×100 ml of ether, and the combined organic phases were dried with Na₂SO₄. After filtration, the solution was concentrated under vacuum in a rotary evaporator, and the residue was purified by flash chromatography on 60 g of silica gel (n-hexane/ethyl acetate=10+1). After drying in an oil-pump vacuum, 0.9741 g (97%) of analytically pure 25 was obtained as a colorless oil.—TLC: n-hexane/ethyl acetate=10+1, R_{f}=0.32.—angles of rotation: [α]₅₈₉²⁰=−49.5, [α]₅₇₈²⁰=−52.5, [α]₅₄₆²⁰=−62.5, [α]₄₃₆²⁰=−136.3, [α]₃₆₅²⁰=−282.7, (c=1.1 in n-hexane).—CD: Θ (λ)=−35370 (227.5 nm), (c=0.002 in n-hexane).—UV (n-hexane): λₘₐₓ(ε)=226.0 nm (15664).—FT-IR (film): \tilde{ν}=3092 (w, unsat. C—H), 2946, 2866 (s,s, sat. C—H), 1597 (m, C═C), 1465, 1367, 1251, 1194, 1140 (m,m,s,m,s), 1066 (s, C—O), 960, 914, 832, 778 (m,m,s,m).—¹H NMR: (270 MHz, C₆D₆): δ=0.09+0.11 (s+s, each 3H, (CH₃)₂Si), 0.89 (ψs, 6H, (CH₃)₂C), 0.94 (d, J=6.8 Hz, 6H, (CH₃)₂CH), 1.63 (sept, J=6.9 Hz, 1H, (CH₃)₂CH), 3.29 (s, 3H, OMe), 3.58 (dd, ³J=3.7 Hz, ²J=11.1 Hz, 1H, CH₂O), 3.79 (ψdd, ³J=2.7 Hz, ²J=10.9 Hz, CH₂O), 4.93 (d, J=11.2 Hz, 1H, 2′-H(E)), 4.97-5.02 (m, 1H, 5-H), 5.02 (d, J=17.7 Hz, 1H, 2′-H(Z)), 5.57 (ψs, 1H, 3-H), 5.87 (ψd, J=4.0 Hz, 1H, 2-H), 6.20 (dd, ³J=10.9 Hz, ³J_{1′,2′(Z)}=18.1 Hz, 1H, 1′-H).—¹³C NMR: (62.90 MHz, C₆D₆): δ=−3.4, −3.3 (each q, (CH₃)₂Si), 18.7, 18.8 (each q, (CH₃)₂CH), 20.5, 20.6 (each q, (CH₃)₂C), 25.4 (s, Si—C), 34.6 (d, (CH₃)₂CH), 53.3 (q, OCH₃), 64.8 (t, CH₂O), 85.6 (d, C-5), 108.6 (d, C-2), 117.6 (t, C-2′), 126.2 (d, C-3), 129.5 (d, C-1′), 143.5 (s, C-4).—Analysis: C₁₆H₃O₃Si (298.50); calc.: C: 64.38%, H: 10.13%; found: C: 64.350%, H: 10.13%.

5. Preparation of (−)-(2′S,5′S)-3-E-(5′-((dimethylthexylsilyloxy)methyl)-2′-methoxy-2′,5′-dihydrofuran-4-yl)acrylic acid ethyl ester (26)

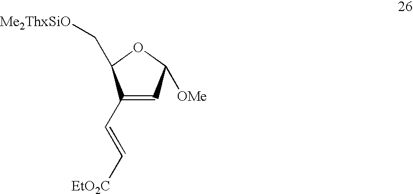

In a 250 ml two-necked flask equipped with a reflux condenser and Hg bubbler headpiece, 1.64 g (5.46 mmol) of aldehyde 24 and 2.85 g (8.18 mmol) of ethoxycarbonylmethylenetriphenylphosphoranine were dissolved in 100 ml of abs. THF and heated to boil in a water bath with magnetic stirring. Since the TLC reaction monitoring showed complete conversion after 1 h (conditions see below); the solution was allowed to cool down to room temperature, concentrated under vacuum in a rotary evaporator and shortly dried in an oil-pump vacuum. The remaining residue was charged onto silica gel with CH₂Cl₂ and purified by flash chromatography on 80 g of silica gel (n-hexane/ethyl acetate=6+1) to obtain 1.92 g (95%) of ester 26 as a yellow oil in an analytically pure form.—TLC: n-hexane/ethyl acetate=4+1, R_{f}=0.39.—angles of rotation: [α]₅₈₉²⁰=−16.8, [α]₅₇₈²⁰=−18.1, [α]₅₄₆²⁰=−23.3, [α]₄₃₆²⁰=−66.5, [α]₃₆₅²⁰=−171.8, (c=1.2 in n-hexane).—CD: Θ (λ)=+1631 (224.0 nm), −16944 (253.5 nm), (c=0.002 in n-hexane).—UV (n-hexane): λₘₐₓ(ε)=253.5 nm (19913).—FT-IR (film): \tilde{ν}=3080 (w, unsat. C—H), 2957, 2868 (m,m, sat. C—H), 1720 (s, C═O), 1648, 1609 (m,m, C═C), 1465, 1366, 1307, 1254, 1177 (m,m,m,m,s), 1041 (s, C—O), 833, 778 (s,m).—¹H NMR: (270 MHz, C₆D₆): δ=0.04+0.06 (s+s, each 3H, (CH₃)₂Si), 0.85 (ψs, 6H, (CH₃)₂C), 0.90 (d, J=6.9 Hz, 6H, (CH₃)₂CH), 0.98 (t, J=7.1 Hz, 3H, OCH₂CH₃), 1.59 (sept, J=6.9 Hz, 1H, (CH₃)₂CH), 3.22 (s, 3H, OMe), 3.53 (dd, ³J=3.2 Hz, ²J=11.1 Hz,1H, CH₂O), 3.65 (dd, ³J=3.5 Hz, ²J=11.1 Hz, 1H, CH₂O), 4.02 (q, J=7.1 Hz, 2H, OCH₂CH₃), 4.85-4.89 (m, 1H, 5′-H), 5.67 (ψs, 1H, 3′-H), 5.75 (□d, J=4.1 Hz, 1H, 2′-H),5.99 (d, J=16.2 Hz, 1H, 2-H), 7.46 (d, J=16.2 Hz, 1H, 3-H).—¹³C NMR: (62.90 MHz, C₆D₆): δ=−3.5, −3.4 (each q, (CH₃)₂Si), 14.2 (q, CH₃CH₂O), 18.7 (q, (CH₃)₂CH), 20.5 (q, (CH₃)₂C), 25.4 (s, Si—C), 34.5 (d, (CH₃)₂CH), 53.5 (q, OCH₃), 60.4 (t, CH₃CH₂O), 64.5 (t, CH₂O), 85.4 (d, C-5′), 108.3 (d, C-2′), 122.5 (d, C-3′), 132.4 (d, C-3), 135.8 (d, C-2), 142.0 (s, C-4′), 165.9 (s, C-1).—Analysis: C₁₉H₃₄O₅Si (370.56); calc.: C: 61.58%, H: 9.250%; found: C: 61.77%, H: 9.27%.

6. Preparation of (−)-endo-[1′,2′,3,4-η-((2S,5S)-5-((dimethylthexylsilyloxy)methyl)-2-methoxy-4-(s-cis-vinyl)-2,5-dihydrofuran)]tricarbonyliron (27)

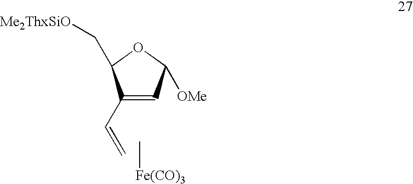

In a 100 ml two-necked flask equipped with a reflux condenser and Hg bubbler headpiece, 0.2425 g (0.81 mmol) of vinyldihydrofuran 25 and 0.44 g (1.21 mmol) of Fe₂(CO)₉ was charged in 40 ml of degassed ether (freshly filtered through basic Al₂O₃) in an argon atmosphere and heated to boil under substantial exclusion of light (turned green by forming Fe₃(CO)₁₂). After 2 h, another addition of 0.15 g (0.41 mmol) of Fe₂(CO)₉ was performed under an argon countercurrent, it was rinsed with a little ether, and the solution was heated under reflux until TLC (conditions see below) showed that all iron-containing by-products (probably η²-Fe(CO)₄ complexes) had disappeared (after a total of 6.5 h). For processing, the reaction solution was concentrated under vacuum in a rotary evaporator, shortly dried in an oil-pump vacuum, then transferred into a flash column with a little n-hexane with strict exclusion of oxygen and subjected to chromatography under argon pressure (90 g of degassed silica gel, first n-hexane for removing Fe(CO)₅ and Fe₃(CO)₁₂, then n-hexane/ethyl acetate=10+1). After drying in an oil-pump vacuum, 0.3091 g (87%) of raw product was obtained in the form of a red-brown oil, which, as seen from ¹H NMR, was a mixture of the endo-complex 27 and itsexo-diastereomer in a ratio of 77:23. The diastereomeric by-product could be separated off by preparative HPLC to isolate 0.1316 g (37%) of complex 27 as a red-brown oil in an analytically pure form.

TLC: n-hexane/ethyl acetate=10+1, R_{f}=0.39.—analyt. HPLC: n-hexane/Ether=10+0.25; MN Nucleosil 50-10; 2 ml/min; refractometry; retention time: 5.64 min.—semiprep. HPLC: n-hexane/ethyl acetate=10+0.3; 10 ml/min; RI detection—angles of rotation: [α]₅₈₉²⁰=−62.2, [α]₅₇₈²⁰=−66.7, [α]₅₄₆²⁰=−82.9, at 436 and 365 nm no transmission of light, (c=1.3 in CHCl₃).—CD: Θ (λ)=−9215 (258.5 nm), +1920(284.0 nm), −7087 (320.0 nm), (c=0.010 in CHCl₃).—UV (CHCl₃): λₘₐₓ(ε)=289.0 nm (2554).—FT-IR (film): \tilde{ν}=3046 (w, unsat. C—H), 2958, 2866 (m,m, sat. C—H), 2054, 1979 (s, s and br., C≡O), 1465, 1365, 1253, 1121 (each m), 1054 (m, C—O), 831, 778 (m,m).—¹H NMR: (270 MHz, CDCl₃): δ=−0.15 (dd, ²J_{2′anti,2′syn}=2.3 Hz, ³J_{2′anti,1′}=8.6 Hz,1H, 2′-Hₐₙₜᵢ), 0.12+0.13 (s+s, each 3H, (CH₃)₂Si), 0.86 (ψs, 6H, (CH₃)₂C), 0.88 (d, J=6.9 Hz, 6H, (CH₃)₂CH), 1.55-1.71 (m, inter alia, with ³J_{2′syn,1′}=6.5 Hz, 2H, 2′-H_{syn}, (CH₃)₂CH), 1.77 (ψd, J=3.0 Hz, 1H, 3-H), 3.42 (s, 3H, OMe), 3.73 (dd, ³J=5.9 Hz, ²J=10.3 Hz, 1H, CH₂O), 3.86 (dd, ³J=3.6 Hz, ²J=10.3 Hz, 1H, CH₂O), 4.81 (ψt, J=4.8 Hz, 1H, 5-H), 5.40-5.41(m, 1H, 2-H), 5.44 (ψt, J=8.1 Hz, 1H, 1′-H).—¹³C NMR: (100.61 MHz, CDCl₃): δ=−3.5, −3.4 (each q, (CH₃)₂Si), 18.6 (q, (CH₃)₂CH), 20.1, 20.3 (each q, (CH₃)₂C), 25.2 (s, Si—C), 34.1 (d, (CH₃)₂CH), 36.3 (t, C-2′), 56.1 (q, OCH₃), 62.5 (d, C-3), 66.4 (t, CH₂O), 74.4 (d, C-1′), 82.5 (d, C-5), 107.9 (d, C-2), 111.5 (s, C-4), 210.2 (br., Fe(CO)₃).—Analysis: C₁₉H₃₀FeO₆Si (438.38); calc.: C: 52.06% H: 6.90%; found: C: 52.04%, H: 6.76%.

7. Preparation of (−)-endo-[2,3,3′,4′-η-(2′S,5′S)-3-E-(5′-((dimethylthexylsilyloxy)methyl)-2′-methoxy-2′,5′-dihydrofuran-4-yl)acrylic acid ethyl ester]tricarbonyliron (28)

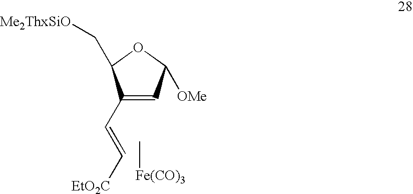

In a 100 ml two-necked flask equipped with a reflux condenser and Hg bubbler headpiece, 0.2467 g (0.67 mmol) of ester 26 and 0.36 g (0.99 mmol) of Fe₂(CO)₉ was charged in 50 ml of degassed ether (freshly filtered through basic Al₂O₃) in an argon atmosphere and heated to boil with substantial exclusion of light (turned green by forming Fe₃(CO)₁₂). After 2 h, another addition of 0.16 g (0.44 mmol) of Fe₂(CO)₉ was performed under an argon countercurrent, it was rinsed with a little ether, and the solution was heated under reflux until TLC (conditions see below) showed that all iron-containing by-products had disappeared (after a total of 28 h). For separating off insoluble Feₓ(CO)_{y} complexes, the reaction solution was filtered under protective gas through 30 g of silica gel, the residue was washed with n-hexane/ethyl acetate=1+1, and the solvent was removed under vacuum in a rotary evaporator. The dark oily residue was subsequently transferred into a flash column with a little n-hexane with strict exclusion of oxygen and subjected to chromatography under argon pressure (hexane/ethyl acetate=8+1). After drying in an oil-pump vacuum, 0.3115 g (92%) of raw product was obtained in the form of a red-brown oil, which, as seen from ¹H NMR, was a mixture of 28 and its exo-diastereomer in a ratio of 79:21. The separation of the diastereomeric by-product was performed by semi-preparative HPLC (conditions see below) to obtain 0.1938 g (57%) of the endo-complex 28 as a red-brown oil in an analytically pure form.

—TLC: n-hexane/ethyl acetate=10+1, R_{f}=0.29.—analyt. HPLC: n-hexane/ethyl acetate=10+0.5; MN Nucleosil 50-10; 2 ml/min; refractometry; retention time: 4.56 min.—semiprep. HPLC: n-hexane/ethyl acetate=10+0.5; 10 ml/min; RI detection—angles of rotation: [α]₅₈₉²⁰=−111.0, [α]₅₇₈²⁰=−117.0, [α]₅₄₆²⁰=−136.8, at 436 and 365 nm no transmission of light, (c=1.2 in CHCl₃).—CD: Θ (λ)=ca. −25985 (256.0 nm),+11968 (314.5 nm), −10338 (350.0 nm), +1566 (402.5 nm), (c=0.011 in CHCl₃).—UV (CHCl₃): λₘₐₓ(ε)=301.0 nm (2883).—FT-IR (film): 3070 (w, unsat. C—H), 2959, 2867 (m,m, sat. C—H), 2063, 1986 (s, s and br., C≡O), 1709 (m, C═O), 1466, 1368, 1253, 1194, 1125, 1052, 833 (each m).—¹H NMR: (270 MHz, CDCl₃): δ=0.11+0.12 (s+s, each 3H, (CH₃)₂Si), 0.59 (d, J=7.5 Hz, 1H, 2-H), 0.84 (ψs, 6H, (CH₃)₂C), 0.87 (d, J=6.9 Hz, 6H, (CH₃)₂CH), 1.25 (t, J=7.1 Hz, 3H, OCH₂CH₃), 1.62 (sept, J=6.9 Hz, 1H, (CH₃)₂CH), 2.03-2.04 (m, 1H, 3′-H), 3.42 (s, 3H, OMe), 3.78 (dd, ³J=5.2 Hz, ²J=10.5 Hz, 1H, CH₂O), 3.88 (dd, ³J=3.3 Hz, ²J=10.5 Hz,1H, CH₂O), 4.02-4.23 (m, inter alia, with J=7.2 Hz, 2H, OCH₂CH₃), 4.79-4.82 (m, 1H, 5′-H), 5.44-5.45 (m, 1H, 2′-H), 5.96 (d, J=8.2 Hz, 1H, 3-H).—¹³C NMR: (100.61 MHz, CDCl₃): δ=−3.5 (q, (CH₃)₂Si), 14.2 (q, CH₃CH₂O), 18.5, 18.6 (each q, (CH₃)₂CH), 20.2, 20.3 (each q, (CH₃)₂C), 25.2 (s, Si—C), 34.1 (d, (CH₃)₂CH), 43.4 (d, C-2), 56.2 (q, OCH₃), 60.5 (t, CH₃CH₂O), 63.1 (d, C-3′), 66.2 (t, CH₂O), 75.4 (d, C-3), 82.6 (d, C-5′), 107.7 (d, C-2′), 110.1 (s, C-4′), 172.4 (s, C-1), 212 (br., Fe(CO)₃).—Analysis: C₂₂H₃₄FeO₈Si (510.44); calc.: C: 51.77%, H: 6.71%; found: C: 51.97%, H: 6.57%.

8. Preparation of exo-[1″,2″,2′,3′-η-(2′,3′-didehydro-2′,3′-dideoxy-5′-O-(dimethylthexylsilyl)-3′-s-cis-vinyl-β-D-uridine]tricarbonyliron (29)

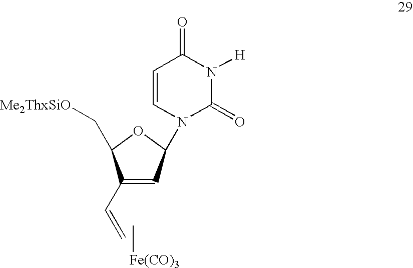

In a baked-out 100 ml three-necked flask with a reflux condenser and Hg bubbler headpiece, 0.3260 g (0.74mmol) of complex 27 and 100 ml of CH₂Cl₂ (freshly filtered through basic Al₂O₃ and degassed) were added to 0.5715 g (2.23 mmol) of 2,4-bis(trimethylsiloxy)pyrimidine with strict exclusion of oxygen and moistureand heated to boil with magnetic stirring. To this solution was added 1.75 ml (1.75 mmol) of SnCl₄ solution (1 M in heptane) within 2 h by means of a metering pump, and it was allowed to cool down to room temperature after the TLC reaction monitoring (conditions see below) showed complete conversion. For processing, the solution was transferred into a separation funnel with 20 ml of CH₂Cl₂ and quickly washed with 100 ml each of saturated aqueous NaHCO₃ solution and saturated aqueous NaCl solution. The aqueous phases were extracted with 2×50 ml of CH₂Cl₂, and the combined organic phases were dried with Na₂SO₄. To avoid possible oxidative decomplexing, the solution was immediately degassed by briefly applying a slight oil-pump vacuum, and then vacuum was broken with argon. After filtration, the solution was concentrated under vacuum in a rotary evaporator and shortly dried in an oil-pump vacuum. Subsequently, the yellow oily residue was transferred into a flash column with strict exclusion of oxygen and subjected to chromatography under argon pressure (90 g degassed silica gel,: n-hexane/ethyl acetate=3+2). After drying in a high vacuum, 0.3617 g (94%) of raw product was obtained in the form of a yellow foam which, as seen from ¹H NMR, was a mixture of complex 29 and its diastereomer in a ratio of 84:16.

The separation of the diastereomeric by-product was effected by semi-preparative HPLC (conditions see below) to obtain 0.2588 g (67%) of complex 29 and 0.0451 as a yellow congealed foam in an analytically pure form.

Melting point: 76-78° C. (CH₂Cl₂), yellow solid foam.—TLC: n-hexane/ethyl acetate=1+1, R_{f}=0.40.—analyt. HPLC: n-hexane/ethyl acetate=10+4.33; MN Nucleosil 50-10; 2 ml/min; refractometry; retention time: 4.65 min.—semiprep. HPLC: n-hexane/ethyl acetate=10+5; 10 ml/min; RI detection—angles of rotation: [α]₅₈₉²⁰=−165.7, [α]₅₇₈²⁰=−175.1, [α]₅₄₆²⁰=−206.8, at 436 and 365 nm no transmission of light, (c=0.9 in MeOH).—CD: Θ (λ)=−1079 (271.5 nm), −21924 (306.0 nm), (c=0.006 in MeOH).—UV (MeLOH): λₘₐₓ(ε)=260.0 nm (14251).—FT-IR (KBr): \tilde{ν}=3412, 3192 (w,w, each br., N—H), 3061 (w, unsat. C—H), 2959, 2867 (m,w, sat. C—H), 2057, 1987 (s, s and br., C≡O), 1686 (s and br., C═O), 1464, 1255, 1123, 832 (each m).—¹H NMR: (270 MHz, CDCl₃): δ=−0.03 (dd, ²J_{2″anti,2″syn}=2.4 Hz, ³J_{2″anti,1″}=8.6 Hz, 1H, 2″-Hₐₙₜᵢ), 0.15+0.18 (s+s, each 3H, (CH₃)₂Si), 0.86-0.88 (m, 12H, (CH₃)₂C, (CH₃)₂CH), 1.63 (sept, J=7.0 Hz, 1H, (CH₃)₂CH), 1.79-1.82 (m, 2H, 2′-H, 2″-H_{syn}) 3.94-4.05 (m, 2H, 5′-H), 4.99 (ψt, J=3.8 Hz, 1H, 4′-H), 5.59 (ψt, J₃=7.8 Hz, 1H, 1″-H), 5.67 (dd, J=2.2 Hz, ³J_{5,6}=8.2 Hz, 1H, 5-H), 6.15 (s, 1H, 1′-H), 7.85 (d, J=8.2 Hz, 1H, 6-H), 8.28 (broad s, 1H, D₂O exchangeable, N—H).—¹³C NMR: (62.90 MHz, CDCl₃): δ=. −3.4, −3.3 (each q, (CH₃)₂Si), 18.5, 18.6 (each q, (CH₃)₂CH), 20.2, 20.3 (each q, (CH₃)₂C), 25.5 (s, Si—C), 34.0 (d, (CH₃)₂CH), 37.6 (t, C-2″), 58.2 (d, C-2′), 66.3 (t, C-5′), 75.6 (d, C-1″), 85.0 (d, C-4′), 90.4 (d, C-1′), 101.9 (d, C-5), 109.7 (s, C-3′), 140.3 (d, C-6), 150.2 (s, C-4), 163.0 (s, C-2), 209.3 (br., Fe(CO)₃).—MS: (MAT 8222, EI, 70 eV): m/z (%)=518 (2) [M⁺], 490 (1) [M⁺-CO], 434 (40) [M⁺-3×CO],349 (48) [M⁺-3×CO, -thexyl].—Analysis: C₂₂H₃₀FeN₂0₇Si (518.42); calc.: C: 50.97%, H: 5.83%, N: 5.40%; found: C: 50.95%, H: 5.67%, N: 5.66%.

9. Preparation of exo-[1″,2″,2′,3′-η-(2′,3′-didehydro-2′,3′-dideoxy-5′-O-(dimethylthexylsilyl)-3′-s-cis-vinyl-β-D-thymidine]tricarbonyliron (30)

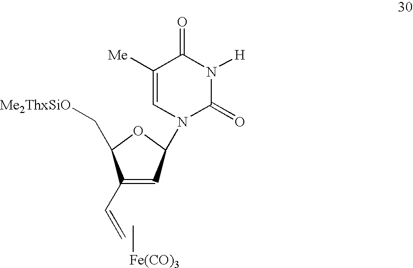

In a baked-out 100 ml two-necked flask with an Hg bubbler, 0.1019 g (0.23 mmol) of complex 28 and 50 ml of CH₂Cl₂ (freshly filtered through basic Al₂O₃ and degassed) were added to 0.1842 g (0.68 mmol) of 5-methyl-2,4-bis(trimethylsiloxy)pyrimidine with strict exclusion of oxygen and moisture and cooled in an ice bath to 0° C. with magnetic stirring. To this solution was added 0.70 ml (0.70 mmol) of SnCl₄ solution (1 M in heptane) within 1.5 h by means of a metering pump, the conversion was monitored by TLC (conditions see below), and then the contents of the flask were transferred into 100 ml of a vigorously stirred saturated aqueous NaHCO₃ solution with a transferring syringe. Subsequently, the solution was transferred to a separation funnel with 20 ml of CH₂Cl₂ and quickly washed with 100 ml of saturated aqueous NaCl solution. The rest of the processing was effected by analogy with that described in paragraph 7.8.3.1. After filtration from the desiccant and removal of the solvent under vacuum in a rotary evaporator, the residue was transferred into a flash column with strict exclusion of oxygen and subjected to chromatography under argon pressure (80 g of degassed silica gel, n-hexane/ethyl acetate=3+2). After drying in an oil-pump vacuum, 0.12379 (100%) of raw product was obtained in the form of a yellow foam which, as seen from ¹H NMR, was a mixture of complex 30 and its diastereomer in a ratio of 59:41. The separation of the diastereomeric by-product was effected by semi-preparative HPLC (conditions see below) to obtain 0.0619 g (50%) of complex 30 as a yellow congealed foam in an analytically pure form.

Complex E14:—Melting point: 74-77° C. (subl., CH₂Cl₂), yellow solid foam.—TLC: n-hexane/ethyl acetate=1+1, R_{f}=0.48.—analyt. HPLC: n-hexane/ethyl acetate=10+4.33; MN Nucleosil 50-10; 2 ml/min; refractometry; retention time: 2.85 min.—semiprep. HPLC: n-hexane/ethyl acetate=10+5; 10 ml/min; RI detection—angles of rotation: [α]₅₈₉²⁰=−176.5, [α]₅₇₈²⁰=−186.5, [α]₅₄₆²⁰=−219.8, [α]₄₃₆²⁰=−468.1, at 365 nm no transmission of light (c=0.9 in MeOH).—CD: Θ (λ)=ca. −9964 (241.5 nm), −12588 (257.0 nm), −8268 (278.0 nm), −22366 (305.0 nm), (c=0.005 in MeOH).—UV (MeOH): λₘₐₓ(ε)=266.0 nm (13387).—FT-IR (KBr): \tilde{ν}=3413, 3185 (w,w, each br., N—H), 3059 (w, unsat. C—H), 2959, 2867 (m,w, sat. C—H), 2057, 1987 (s, s and br., C≡O), 1691 (s and br., C═O), 1466, 1255, 1118, 832 (each m).—¹H NMR: (250 MHz, CDCl₃): δ=0.00 (dd, ²J_{2″anti,2″syn}=2.5 Hz, ³J_{2″anti,1″}=8.7 Hz, 1H, 2″-Hₐₙₜᵢ), 0.16+0.17 (s+s, each 3H, (CH₃)₂Si), 0.88-0.90 (m, 12H, (CH₃)₂C, (CH₃)₂CH), 1.65 (sept, J=6.9 Hz, 1H, (CH₃)₂CH), 1.79-1.83 (m, inter alia, with J=2.5 Hz, 2H, 2″-H_{syn}, 2′-H), 1.92 (d, ⁴J=1.2 Hz, 3H, 5-CH₃), 3.84 (dd, ³J_{5′a,4′}=6.2 Hz, ²J_{5′a,5′b}=10.8 Hz, 1H, 5′-Ha), 3.98 (dd, ³J_{5′b,4′}=4.8 Hz, ²J_{5′b,5′a}=10.8 Hz, 1H, 5′-Hb), 4.92 (ψt, J=5.2 Hz, 1H, 4′-H), 5.64 (ψt, J=7.9 Hz, 1H, 1″-H), 6.09 (s, 1H, 1′-H), 7.35 (d, ⁴J=1.2 Hz, 1H, 6-H), 8.40 (broad s, 1H, D₂O exchangeable, N—H).—¹³C NMR: (62.90 MHz, CDCl₃): δ=−3.3 (q, (CH₃)₂Si), 12.7 (q, 5-CH₃), 18.5, 18.6 (each q, (CH₃)₂CH), 20.2, 20.4 (each q, (CH₃)₂C), 25.4 (s, Si—C), 34.0 (d, (CH₃)₂CH), 37.6 (t, C-2″), 57.5 (d, C-2′), 66.3 (t, C-5′), 76.2 (d, C-1″), 84.5 (d, C-4′), 90.8 (d, C-1′), 109.8 (s, C-3′), 110.3 (s, C-5), 135.5 (d, C-6), 150.3 (s, C-4), 163.5 (s, C-2), 209.5 (br., Fe(CO)₃).—MS: (MAT 8222, EI, 70 eV): m/z (%)=532 (3) [M⁺], 504 (1) [M⁺-CO], 448 (40) [M⁺-3×CO], 363 (50) [M⁺-3×CO, −thexyl].—Analysis: C₂₃H₃₂FeN₂O₇Si (532.45); calc.: C: 51.88%, H: 6.06%, N: 5.26%; found: C: 51.96%, H: 6.11%, N: 5.19%.

10. Preparation of exo-[2′,2″,3′,3″-η-(3′-(3″(E)-acrylic-acid-ethyl-ester-3-yl)-2′,3′-didehydro-2′,3′-dideoxy-5′-O-(dimethylthexylsilyl)-f3b-D-uridine)]tricarbonyliron (31)

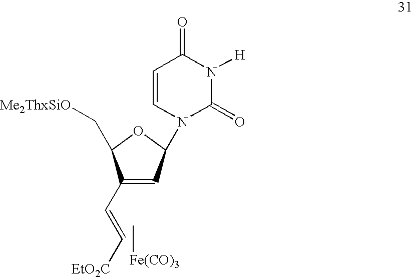

With strict exclusion of oxygen and moisture, 0.2495 g (0.97 mmol) of 2,4-bis(trimethylsiloxy)pyrimidine, 0.1774 g (0.35 mmol) of ester 28 and 80 ml of CH₂Cl₂ were heated to boil with magnetic stirring. By means of a metering pump, 1.20 ml (1.20 mmol) of SnCl₄ solution (1 M in heptane) was added within 2 hours. After the TLC reaction monitoring (conditions see below) showed that conversion was complete, processing was effected as described above. After filtration from the desiccant and removal of the solvent under vacuum in a rotary evaporator, the residue was transferred into a flash column with strict exclusion of oxygen and subjected to chromatography under argon pressure (80 g of degassed silica gel, n-hexane/ethyl acetate=1+1). After drying in an oil-pump vacuum, 0.1968 g (96%) of raw product was obtained in the form of a yellow foam which, as seen from ¹H NMR, was a mixture of complex 31 and its diastereomer in a ratio of 87:13. The separation of the diastereomeric by-product was effected by semi-preparative HPLC (conditions see below) to obtain 0.1497 g (73%) of the complex as a yellow congealed foam in an analytically pure form.

Melting point: 84-86° C. (CH₂Cl₂), yellow solid foam.—TLC: n-hexane/ethyl acetate=1+1, R_{f}=0.40.—analyt. HPLC: n-hexane/ethyl acetate=10+6.67; MN Nucleosil 50-10; 2 ml/min; refractometry; retention time: 3.75 min.—semiprep. HPLC: n-hexane/ethyl acetate=10+6; 10 ml/min; RI detection—angles of rotation: [α]₅₈₉²⁰=−182.0, [α]₅₇₈²⁰=−190.4, [α]₅₄₆²⁰=−217.4, at 436 and 365 nm no transmission of light (c=1.0 in MeOH).—CD: Θ (λ)=ca. −10310 (272.5 nm),−14553 (288.0 nm), −2003 (316.5 nm), −9848 (344.0 nm), +3878 (397.0 nm), (c=0.007 in MeOH).—UV (MeOH): λₘₐₓ(ε)=300.0 (3042).—FT-IR (KBr): \tilde{ν}=3413, 3203 (w,w, each br., N—H), 3062 (w, unsat. C—H), 2960, 2868 (m,w, sat. C—H), 2067, 2004 (s, s and br., C≡O), 1704 (s and br., C═O), 1465, 1378, 1268, 831 (each m).—¹H NMR: (270 MHz, CDCl₃): δ=0.15+0.17 (s+s, each 3H, (CH₃)₂Si), 0.73 (d, J³=7.6 Hz, 1H, 2″-H), 0.85-0.87 (m, 12H, (CH₃)₂C, (CH₃)₂CH), 1.26 (t, J=7.1 Hz, 3H, OCH₂CH₃), 1.62 (sept, J=6.8 Hz, 1H, (CH₃)₂CH), 2.10 (s, 1H, 2′-H), 3.99-4.04 (m, 2H, 5′-H), 4.06-4.25 (m, inter alia, with J=7.1 Hz, 2H, OCH₂CH₃), 4.98 (ψt, J=3.8 Hz, 1H, 4′-H), 5.69 (dd, J=2.3 Hz, ³J_{5,6}=8.1 Hz,1H, 5-H), 6.10 (dd, J=0.7 Hz, ³J_{3″,2″}=8.4 Hz, 1H, 3″-H), 6.11 (s, 1H, 1′-H), 7.85 (d, J=8.1 Hz, 1H, 6-H), 8.43 (broad s, 1H, D₂O exchangeable, N—H).—¹³C NMR: (62.90 MHz, CDCl₃): δ=−3.4, −3.2 (each q, (CH₃)₂Si), 14.1 (q, CH₃CH₂O), 18.4, 18.5 (each q, (CH₃)₂CH), 20.2, 20.3 (each q, (CH₃)₂C), 25.4 (s, Si—C), 33.9 (d, (CH₃)₂CH), 44.9 (d, C-1″), 58.6 (d, C-2′), 60.9 (t, CH₃CH₂O), 66.1 (t, C-5′), 76.5 (d, C-3″), 85.1 (d, C-4′), 90.4 (d, C-1′), 102.0 (d, C-5), 108.2 (s, C-3′), 140.1 (d, C-6), 150.1 (s, C-4), 162.6 (s, C-2), 171.5 (s, C-1″), 209.5 (br., Fe(CO)₃).—MS: (MAT 8222, EI, 70 eV): m/z (%)=590 (<1) [M⁺], 545 (3) [M⁺—OCH₂CH₃], 506 (100) [M⁺-3×CO], 421 (50) [M⁺-Fe(CO)₃, −OCH₂CH₃].—Analysis: C₂₅H₃₄FeN₂O₉Si (590.48); calc.: C: 50.85%, H: 5.80%, N: 4.74%; found: C: 50.64%, H: 5.86%, N: 4.70%.

11. Preparation of exo-[2′,2″,3′,3″-η-(3′-(3″(E)-acrylic-acid-ethyl-ester-3-yl)-2′,3′-didehydro-2′,3′-dideoxy-5′-O-(dimethylthexylsilyl)-f3b-D-thymidine)]tricarbonyliron (32)

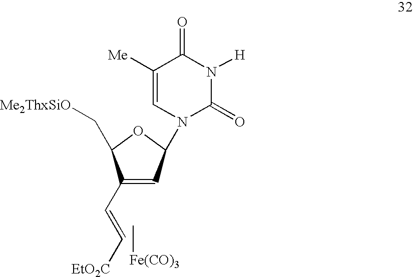

By analogy with the above protocols, 0.2988 g (1.10 mmol) of 5-methyl-2,4-bis(trimethylsiloxy)pyrimidine, 0.1464 g (0.29 mmol) of ester 28 and 70 ml of CH₂Cl₂ were heated to boil with magnetic stirring and with strict exclusion of oxygen and moisture, and 1.30 ml (1.30 mmol) of SnCl₄ solution (1 M in heptane) was added by means of a metering pump within 2 h. After the TLC reaction monitoring (conditions see below) showed that conversion was complete, processing was effected as described above. After filtration from the desiccant and removal of the solvent under vacuum in a rotary evaporator, the residue was transferred into a flash column with strict exclusion of oxygen and subjected to chromatography under argon pressure (80 g of degassed silica gel, n-hexane/ethyl acetate=1+1). After drying in an oil-pump vacuum, 0.1609 g (93%) of raw product was obtained in the form of a yellow foam which, as seen from ¹H NMR, was a mixture of complex 32 and a diastereomer in a ratio of 70:30. The separation of the diastereomeric by-product was effected by semi-preparative HPLC (conditions see below) to obtain 0.0983 g (57%) of complex 32 as a yellow congealed foam in an analytically pure form.

Melting point: 81-84° C. (CH₂Cl₂), yellow solid foam.—TLC: n-hexane/ethyl acetate=1+1, R_{f}=0.45.—semiprep. HPLC: n-hexane/ethyl acetate=160+5; 10 ml/min; RI detection—angles of rotation: [α]₅₈₉²⁰=−205.5, [α]₅₇₈²⁰=−215.1, [α]₅₄₆²⁰=−246.4, at 436 and 365 nm no transmission of light (c=0.9 in MeOH).—CD: Θ (λ)=−32856 (251.5 nm), −2643 (316.5 nm), −10309 (343.5 nm), +3991 (397.0 nm), (c=0.005 in MeOH).—UV (MeOH): λₘₐₓ(ε)=259.5 (15053), λ(ε)=305.0 (sh, 3032).—FT-IR (KBr): \tilde{ν}=3412, 3190 (w,w, each br., N—H), 3065 (w, unsat. C—H), 2959, 2868 (m,w, sat. C—H), 2067, 2004 (s, s and br., C≡O), 1698 (s and br., C═O), 1466, 1265, 1197, 832 (each m).—¹H NMR: (250 MHz, CDCl₃): δ=0.15+0.17 (s+s, each 3H, (CH₃)₂Si), 0.76 (d, J=7.7 Hz, 1H, 2″-H), 0.87 (s, 6H, (CH₃)₂C), 0.88 (d, J=5.4 Hz, (CH₃)₂CH), 1.26 (t, J=7.1 Hz, 3H, OCH₂CH₃), 1.64 (ψquin, J=6.8 Hz, 1H, (CH₃)₂CH), 1.92 (d, ⁴J=1.2 Hz, 3H, 5-CH₃), 2.14 (s, 1H, 2′-H), 3.90 (dd, ³J_{5′a,4′}=5.7 Hz, ²J_{5′a,5′b}=10.9 Hz, 1H, 5′-Ha), 4.01 (dd, ³J_{5′b,4′}=4.8 Hz, ²J_{5′b,5′a}=10.9 Hz, 1H, 5′-Hb), 4.06-4.23 (m, inter alia with J=7.1 Hz, 2H, OCH₂CH₃), 4.92 (ψt, J=5.3 Hz, 1H, 4′-H), 6.04 (s, 1H, 1′-H), 6.15 (d, J=8.3 Hz, 1H, 3″-H), 7.36 (d, ⁴J=1.2 Hz, 1H, 6-H), 8.41 (broad s, 1H, D₂O exchangeable, N—H).—¹³C NMR: (62.90 MHz, CDCl₃): δ=−3.3 (q, (CH₃)₂Si), 12.7 (q, 5-CH₃), 14.1 (q, CH₃CH₂O), 18.5, 18.6 (each q, (CH₃)₂CH), 20.2, 20.3 (each q, (CH₃)₂C), 25.4 (s, Si—C), 34.0 (d, (CH₃)₂CH), 45.0 (d, C-2″), 58.1 (d, C-2′), 60.8 (t, CH₃CH₂O), 66.1 (t, C-5′), 77.1 (d, C-3″), 84.7 (d, C-4′), 90.9 (d, C-1′), 108.3 (s, C-5), 110.4 (s, C-3′), 135.4 (d, C-6), 150.3 (s, C-4), 163.4 (s, C-2), 171.5 (s, C-1″), 209 (br., Fe(CO)₃).—MS: (MAT 8222, DCI negative, NH₃): m/z (%)=604 (20) [M⁺], 576 (15) [M⁺—CO], 548 (80) [M⁺-2×CO], 265 (100) [M⁺-Fe(CO)₃,—thymine-1-yl,—CO₂Et, - H].—Analysis: C₂₆H₃₆FeN₂O₉Si (604.51); calc.: C: 51.66%, H: 6.00%, N: 4.63%; found: C: 51.82%, H: 6.07%, N: 4.54%.

12. Preparation of exo-[2′,2″,3′,3″-η-(3′-(3″-(E)-acrylic-acid-ethyl-ester-3″-yl)-2′,3′-didehydro-2′,3′-dideoxy-5′-O-(dimethylthexylsilyl)-β-D-cytidine)]tricarbonyliron (6)

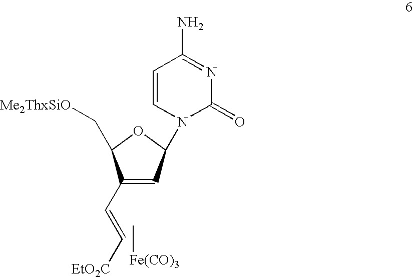

In a 100 ml three-necked flask with a reflux condenser, magnetic stirring bar and suction cock, 0.570 g (1.12 mmol) of complex 28 and 0.672 mg (2.63 mmol) of N,O-bis(trimethylsilyl)cytosine were dissolved in 40 ml of abs. dichloromethane under an argon atmosphere and heated to reflux. Over a period of four hours, a solution of 1.00 ml of TMSOTf (1.24 g, 5.6 mmol) in 8 ml of abs. dichloromethane was metered thereto, and the reaction mixture was subsequently heated under reflux for another hour. After cooling, the contents of the flask were poured onto ice, and saturated NaHCO₃ solution was added. After the phases had separated, the organic phase was washed with water, and the aqueous phases were re-extracted twice with dichloromethane. The combined organic phases were dried over sodium sulfate. After filtering off the desiccant, the solvent was removed in a rotary evaporator under an argon atmosphere. The residue was subjected to chromatography under an argon atmosphere on degassed silica gel with a degassed mixture of solvents of ethyl acetate: methanol=8:1. The product 6 was obtained in a yield of 171 mg (0.29 mmol, 260%) as a congealed yellowish-white foam.—TLC: ethyl acetate:methanol=30:1, R_{f}=0.094.—¹H NMR: (300 MHz, CDCl₃): δ=0.12 (s, 3H, Si(CH₃)), 0.14 (s, 3H, Si(CH₃)), 0.68 (d, 1H, 2″-H, ³J_{2″-H,3″-H}=7.9 Hz), 0.82 (s, 3H, SiC(CH₃)), 0.83 (s, 3H, SiC(CH₃)), 0.84 (_d, 6H, C(CH₃)₂H, ³J_{C(CH3)2H, C(CH3)2H}=6.9 Hz), 1.22 (ψt, 3H, CH₃CH₂, ³J_{CH3CH2, CH3CHH2}=7.2 Hz), 1.59 (ψsept, 1H, C(CH₃)₂H, ³J_{C(CH3)2H, C(CH3)2H}=6.9 Hz), 2.32 (s, 1H, 2′-H), 3.96 (ψd, 2H, 5′-H, ³J_{4′-H, 5′-H}=4.3 Hz), 4.06 (m, 1H, CH₃CHaHb), 4.14 (m, 1H, CH₃CHaHb), 4.95 (ψt, 1H, 4′-H, ³J_{4′-H, 5′-H}=4.3 Hz), 5.64 (d, 1H, 5-H, ³J_{5-H, 6-H}=7.4 Hz), 6.00 (s, very broad, 2H, NH₂), 6.04 (d, 1H, 3″-H, ³J_{2″-H, 3″-H}=7.9 Hz), 6.06 (s, 1H, 1′-H), 7.83 (d, 1H, 6-H, ³H_{5-H, 6-H=}7.4 Hz)—¹³C NMR: (70 MHz, CDCl₃): −3.4 and −3.2 (Si(CH₃)₃), 14.1 (CH₃CH₂), 18.5 and 18.6 (C(CH₃)₂H), 20.2 and 20.3 (SiC(CH₃)₂), 25.4 (SiC(CH₃)₂), 34.0 (C(CH₃)₂H), 44.6 (2″-C), 60.4 (2′-C), 60.7 (CH₃CH₂), 66.2 (5′-C), 76.3 (3″-C), 85.1 (4′-C), 91.8 (1′-C), 93.6 (5-C), 107.9 (3′-C), 141.3 (6-C), 155.8 (2-C), 156.7 (4-C), 171.8 (1″-C).

13. Preparation of exo-[1″,2″,2′,3′-η-(2′,3′-didehydro-2′,3′-dideoxy-3′-s-cis-vinyl-β-D-uridine)]tricarbonyliron (33)

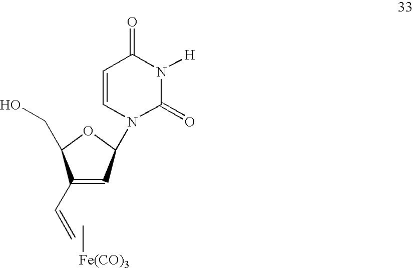

In a 100 ml Schlenk flask with an Hg bubbler headpiece, 0.2515 g (0.48 mmol) of complex 29 in 50 ml of abs. THF was charged under protective gasand cooled to 0° C. with an ice bath. To the magnetically stirred solution, 2.5 ml (2.5 mmol) of TBAF solution (1 M in THF) was injected under an argon countercurrent, and the reaction was allowed to continue for 2.5 h, whereupon TLC reaction monitoring (conditions see below) showed that conversion was complete. For processing, the solution was transferred into a separation funnel with 100 ml of ethyl acetate, and 100 ml of H₂O was added. After the phases had separated, the organic phase was washed with 100 ml of saturated aqueous NaCl solution, the aqueous phases were extracted with 100 ml of ethyl acetate, and the combined organic phases were dried over Na₂SO₄. To avoid possible oxidative decomplexing, the solution was immediately degassed by briefly applying a slight oil-pump vacuum, and then vacuum was broken with argon. After filtration, the solution was concentrated under vacuum in a rotary evaporator and briefly dried in an oil-pump vacuum. Subsequently, the yellow residue was transferred into a flash column with strict exclusion of oxygen and subjected to chromatography under argon pressure (90 g of degassed silica gel, n-hexane/acetone=9+11). After drying in a high vacuum, 0.1812 g (99%) of the desilylated complex 33 was obtained in the form of a yellow solid, which was subsequently recrystallized from CH₂Cl₂ at −20° C.—Melting point: 118-120° C. (CH₂Cl₂), yellow solid.—TLC: n-hexane/acetone=1+1, R_{f}=0.23.—CD: Θ (λ)=−5451 (274.0 nm), −20501 (306.0 nm), (c=0.006 in MeOH). UV (MeOH): λₘₐₓ(ε)=258.5 nm (13326), λ (ε)=300.0 nm (sh, 2384).—FT-IR (KBr): \tilde{ν}=3394, 3198 (m,w, each br., N—H), 3060 (w, unsat. C—H), 2934 (w, sat. C—H), 2057, 1976 (s, s and br., C≡O), 1707, 1683 (each s and br., C═O), 1471, 1258, 1096, 966 (each m).—¹H NMR: (270 MHz, acetone-d⁶): δ=0.32 (dd, ²J_{2″anti,2″syn}=2.0 Hz, ³J_{2″anti,1″}=8.8 Hz, 1H, 2″-Hₐₙₜᵢ), 1.91 (dd, ²J_{2″syn,2″anti}=2.0 Hz, ^{3l J}_{2″syn,1″}=6.7 Hz, 1H2″-H_{syn}), 2.32 (s, 1H, 2′-H), 3.94 (ψdd, ³J_{5′a,4′}=4.3 Hz, ²J_{5′a,5′b}=11.9 Hz, 1H, 5′-Ha), 4.06 (ψdd, ³J_{5′b,4′}=4.1 Hz, ²J_{5′b,5′a}=11.9 Hz, 1H, 5′-Hb), 4.57 (ψt, J=4.9 Hz, non-integratable because in part already exchanged, D₂0 exchangeable, O—H), 5.03 (ψt, J=3.8 Hz, 1H, 4′-H), 5.54 (d, J=8.1 Hz, 1H, 5-H), 6.01 (ψt, J=7.9 Hz, 1H, 1″-H), 6.31 (s, 1H, 1′-H), 8.05 (d, J=8.1 Hz, 1H, 6-H), 10.0 (broad s, non-integratable because in part already exchanged, D₂O exchangeable, N—H).—MS: (MAT 8222, EI, 70 eV): m/z (%)=376 (1) [M⁺], 348 (1) [M⁺-CO], 320 (1) [M⁺-2×CO], 292 (1) [M⁺-3×CO], 262 (35) [M⁺-3×CO, —CH₂×OH, +H], 236 (10) [M⁺-ura., —CO, —H], 208 (18) [M⁺-ura., -2×CO, —H], 180 (40) [M⁺-ura., -3×CO, −H].—HRMS: (MAT 8222, 70 eV): calc.: 375.9994; found: 375.9977; C₁₄H₁₂FeN₂O₇ (376.11).

14. Preparation of 2′,3′-didehydro-2′,3′-dideoxy-3′-vinyl-β-D-uridine (34)

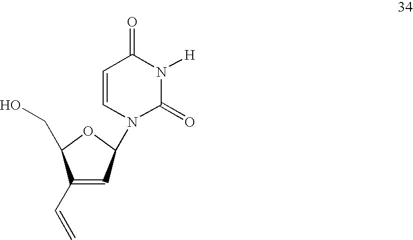

In a 100 ml flask, 0. 1719 g (0.46 mmol) of complex 33 was dissolved in 30 ml of EtOH, and 20 ml of H₂O was added. To the magnetically stirred solution, which was cooled to 0° C., was added 2.10 g (3.8 mmol) of Ce(NH₄)₂(NO₃)₆, and the reaction was allowed to continue for 45 min (immediate evolution of CO). For processing, the reaction solution was transferred into a separation funnel with 80 ml of ethyl acetate, and 100 ml of H₂O was added. After the phases had separated, the organic phase was washed with 100 ml of satuirated aqueous NaCl solution, the aqueous phases were extracted with 2×80 ml of ethyl acetate, and the combined organic phases were dried over Na₂SO₄. After filtration from the desiccant, the solution was concentrated under vacuum in a rotary evaporator and subsequently dried in an oil-pump vacuum to obtain 0.056 g (52%) of the product 34 as a colorless solid in a pure form.—Melting. point: 151° C. (ethyl acetate, decomposition.).—FT-IR (KBr): \tilde{ν}=3442 (m and br., O—H, N—H), 3094 (w, unsat. C—H), 2927 (w, sat. C—H), 1704 (m, C═O), 1467, 1396, 1249 (each m), 1127 (s, C—O).—¹H NMR: (270 MHz, CD₃OD): δ=3.79 (ψd, J=12.5 Hz, 1H, 5′-Ha), 3.88 (ψd, J=12.2 Hz, 1H, 5′-Hb), 5.00 (s, 1H, 4′-H), 5.36 (d, J=11.1 Hz, 1H, 2″-H(E)), 5.47 (d, J=17.9 Hz, 1H, 2″-H(Z)), 5.60 (d, J=7.8 Hz, 1H, 5-H), 5.83 (s, 1H, 2′-H), 6.55 (dd, ³J_{1″,2″}=11.3 Hz, ³J_{1″,2″(Z)}=17.9 Hz, 1H, 1″-H), 6.84 (s, 1H, 1′-H), 7.91 (d, J=7.7 Hz, 1H, 6-H). C₁₁H₁₂N₂O₄ (236.23).

15. Preparation of (5RS,8RS)-8-allyloxy-2-trimethylsilyl-7-oxabicyclo-[3.3.0]oct-1-ene-3-one (rac-38).

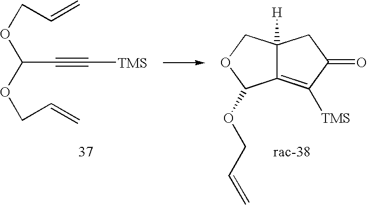

To a stirred mixture of octacarbonyidicobalt (25 g, 69 mmol) and 4 Å molecular sieve (108 g) in dry degassed CH₂Cl₂ (1.5 l), (13.52 g, 60 mmol) of 3,3-di-allyloxy-1-propinyltrimethylsilane (37) was added under argon at room temperature. After stirring at room temperature for 2 h, the reaction mixture was cooled down to <0° C. (ice-NaCl bath), and azeotropically dried trimethylamine-N-oxide (42 g, 560 mmol) was added. After the addition, the flask was left open, and air was passed through the reaction solution for 5-10 min. Subsequently, the solution was allowed to come to room temperature, and stirring was continued for 15 h in the air. The reaction mixture, which was filtered through some silica gel, was concentrated under vacuum, and the residue was purified by flash chromatography (EtOAc/CyHex=1+4) to obtain rac-38 as a pale-yellow oil in a yield of 11.56 g (76%). (diastereoselectivity>24:1).

¹H NMR (250 MHz, CDCl₃): δ=5.93 (tdd, J₁=5.4, J₂=17.2, J₃=10.3, 1H, CH═CH₂); 5.58 (s, 1H, H-8); 5.31 (tdd, H₁=1.6, J₂=17.2, J₃=1.6, 1H, CH═CH-Hₜᵣₐₙₛ); 5.22 (tdd, J₁=1.2, J₂=10.3, J₃=1.8, 1H, CH═CH—-H_{cis}); 4.38 (dd, J₁=6.6, J₂=7.2, 1H, H-6); 4.29 (tdd, J₁=1.4, J₂=12.2, J₃=5.6, 1H, CH₂—CH═CH₂); 4.10 (tdd, J₂=1.3, J₃=12.5, J₄=6.4, 1H, CH₂—CH═CH₂); 3.46 (m, 1H, H-5); 3.42 (dd, J₁=6.7, J₂=6.2, 1H, H-6); 2.66 (dd, J₁=17.6, J₂=6.4, 1H, H-4); 2.10 (dd, J₁=17.6, J₂=3.6, 1H, H-4); 0.21 (s, 9H, SiCH₃); ¹³C NMR (63 MHz, CDCl₃): δ=213.4 (C3), 184.9 (C1), 137.6 (C2), 133.8 (CH═CH₂), 118.2 (CH═CH₂), 96.7 (C8), 71.0 (C6), 68.8 (CH₂CH═CH₂), 42.6 (C5), 41.8 (C4), −1.5 (SiC); FT-IR (ATR): 2954 (m, CH), 2896 (m, CH), 1703 (s, C═O), 1640 (s, C═C), 1410 (m), 1247 (s, C—O), 1126 (s), 1073 (s), 997 (s), 838 (s), 762 (s); MS (EI, 70 eV): m/z (%): 253 (1) [M+1]⁺, 237 (2), 211 (15), 195 (20), 181 (21), 167 (8), 151 (20), 137 (7), 123 (32), 109 (9), 93 (17), 75 (44), 73 (100), 59 (11), 41 (21); HRMS (EI) C₁₂H₁₇O₃Si: calc. 237.095, [M−15]⁺; found 237.095.

16. Preparation of (3RS,5RS,8RS)-8-allyloxy-2-trimethylsilyl-7-oxabicyclo[3.3.0]oct-1-ene-3-ol (rac-39).

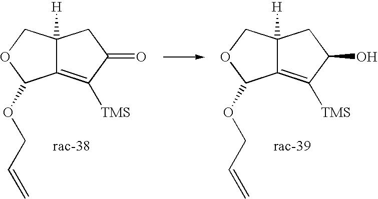

To an ice-cooled solution (ca. 0° C.) of rac-38 (2.52 g, 10 mmol) and CeCl₃.7H₂O (3.54 g, 9.5 mmol) in MeOH (200 ml), NaBH₄ (1.48 g, 38 mmol) was added in small portions, the solution was allowed to come to room temperature, and stirring was continued for 0.5 h before the reaction was quenched by the addition of water (100 ml). After 1 h, the solution was partitioned between MTBE (100 ml) and 10% NaHCO₃ (aq.) (100 ml), and the aqueous phase was again extracted with MTBE (3×100 ml). The combined organic phases were washed with 10% NaHCO₃ (aq.) (100 ml) and sat. NaCl (aq.) (100 ml), dried with MgSO₄, filtered, and the solvent was removed under vacuum to obtain rac-39 as a colorless oil (2.46 g, 98%) which, as seen from NMR, did not require further purification and thus was directly reacted further. ¹H NMR (250 MHz, CDCl₃): δ=5.90 (tdd, J₁=5.4, J₂=17.2, J₃=10.3, 1H, CH═CH₂); 5.38 (s, 1H, H-8); 5.26 (tdd, J₁=1.3, J₂=17.2, J₃=1.5, 1H, CH═CH—Hₜᵣₐₙₛ); 5.17+5.16 (m+tdd, J₁=1.1, J₂=10.3, J₃=1.8, 2H, H-3+CH═CH—Hₕᵢₛ); 4.20 (dd, J₁=J₂=8.2, 1H, H-6); 4.19 (tdd, J₁=1.4, J₂=12.4, J₃=5.3, 1H, CH₂—CH═CH₂); 4.00 (tdd, J₁=1.3, J₂=12.4, J₃=6.4, 1H, CH₂—CH═CH₂); 3.42 (dd, J₁=J₂=7.9, 1H, H-6); 3.20 (m, 1H, H-5); 2.69 (ddd, J₁=12.1, J₂=6.5, J₃=6.0, 1H, H-4); 1.64 (ψs, 1H, OH); 1.22 (ddd, J₁=12.0, J₂=J₃=8.6 Hz, 1H, H-4); 0.21 (s, 9H, SiCH₃); ¹³C NMR (63 MHz, CDCl₃): δ=156.8 (C1), 140.3 (C2), 134.3 (CH═CH₂), 117.7 (CH═CH₂), 97.3 (C8), 87.61 (C3), 72.1 (C6), 68.3 (CH₂CH═CH₂), 46.4 (C5), 43.9 (C4), −0.6 (SiC); FT-R (ATR): 3437 (m, OH), 2952 (s, CH), 2886 (m, CH), 1657 (m, C═C), 1408 (m), 1324 (s), 1245 (s, C—O), 1107 (s), 1064 (s), 989 (s), 834 (s), 753 (s); MS (EI, 70 eV): m/z (%)=253 (1) [M−1]⁺, 210 (3), 197 (22), 183 (20), 167 (17), 151 (19), 135 (11), 123 (20), 115 (7), 107 (18), 95 (16), 75 (46), 73 (100), 59 (10), 41 (21); HRMS (EI) C₁₂H₁₉O₃Si: calc. 239.114 [M−15]⁺; found 239.110.

17. Preparation of (3RS,5RS,8RS)-8-allyloxy-7-oxa-bicyclo[3.3.0]oct-1-ene-3-ol (rac-39′)

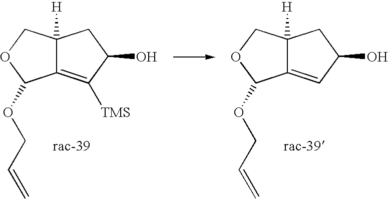

To a stirred mixture of t-BuOK (1.14 g) in DMSO/H₂O (1:19 v/v; ca. 20 ml), rac-39 (2.54 g, 10 mmol) was added at room temperature. The reaction mixture, which was now brown, was heated under reflux for 2 h. After cooling down to room temperature, water (100 ml) was added, and the solution w as extracted with ethyl acetate (4×80 ml). The combined organic phases were washed with water (2×100 ml) and sat. NaCl solution (100 ml), dried with MgSO₄, and the filtered solution was concentrated under vacuum. The product obtained as a pale-yellow oil (rac-39′) (1.59 g, 87%) proved to be sufficiently pure to be directly reacted further. Nevertheless, an analytical sample was further purified by flash chromatography (EtOAc/CyHex=1+9).

¹H NMR (250 MHz, CDCl₃): δ=5.85 (tdd, J₁=5.4, J₂=17.2, J₃=10.3, 1H, CH═CH₂); 5.70 (m, 1H, H-2); 5.36 (s, 1H, H-8); 5.22 (tdd, J₁=1.2, J₂=17.2, J₃=1.6, 1H, CH═CH—Hₜᵣₐₙₛ); 5.13+5.12 (m+tdd, J₁=1.2, J₂=10.3, J₃=1.6, 2H, H-3+CH═CH—H_{cis}); 4.18 (dd, J₁=J₂=8.2, 1H, H-6); 4.12 (tdd, J₁=1.6, J₂=12.6, J₃=5.3, 1H, CH₂—CH═CH₂); 3.96 (tdd, J₁=1.6, J₂=12.6, J₃=6.1, 1H, CH₂—CH═CH₂); 3.38 (dd, J₁=J₃=7.1, 1H, H-6); 3.20 (m, 1H, H-5); 2.66 (ddd, J₁=12.4, J₂=6.3, J₃=6.1, 1H, H-4); 2.11 (ψs, 1H, OH); 1.29 (ddd, J₁=12.3, J₂=J₃=8.0, 1H, H-4); ¹³C NMR (63 MHz, CDCl₃): δ=147.9 (C1), 134.0 (CH═CH₂), 126.8 (C2), 117.3 (CH═CH₂), 96.8 (C8), 82.2 (C3), 72.7 (C6), 67.8 (CH₂CH═CH₂), 44.9 (C5), 42.9 (C4); FT-IR (ATR): 3396 (w, OH), 2920 (s, C—H), 2851 (m, C—H), 1646 (w, C═C), 1458 (m), 1326 (m), 1295 (m, C—O), 1149 (m), 1033 (s), 985 (s), 823 (s), 775 (m); MS (EI, 70 eV): m/z (%): 183 (4) [M+1]⁺, 152 (20), 125 (13), 110 (40), 95 (82), 83 (64), 69 (60), 66 (98), 55 (100).

18. Preparation of (3RS,5RS,8RS)-3-acetoxy-8-allyloxy-7-oxabicyclo-[3.3.0]oct-1-ene (rac-40).

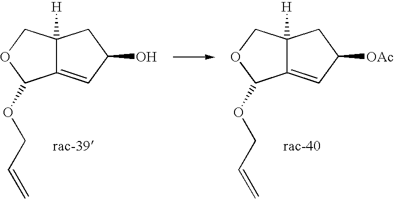

To a mixture of rac-39′ (2.38 g, 13 mmol), Et₃N (2.3 ml, 15.6 mmol) and DMAP (196 mg, 1.56 mmol) in CH₂Cl₂ (50 ml) stirred under argon, acetic anhydride (2.7 ml, 28.6 mmol) was added at room temperature. After 3 h of stirring, sat. NaHCO₃ solution (aq.) (50 ml) was added to the reaction mixture; and the water phase was extracted with CH₂Cl₂ (3×80 ml). The organic phases were washed with sat. solutions of NaHCO₃ (aq.) (2×100 ml) and NaCl (100 ml), dried with MgSO₄, and the solvent was removed under vacuum. The brown residue was purified by flash chromatography (EtOAc/CyHex 1+4) to obtain rac-40 as a colorless oil (2.89 g, 99%).

¹H NMR (250 MHz, CDCl₃): δ=5.95 (m, 1H, H-3); 5.87 (tdd, J₁=5.4, J₂=17.2, J₃=10.3, 1H, CH═CH₂); 5.73 (m, 1H, H-2); 5.41 (s, 1H, H-8); 5.26 (tdd, J₁=1.2, J₂=17.2, J₁=1.6, 1H, CH═CH—Hₜᵣₐₙₛ); 5.17 (tdd, J₁=1.3, J₂=10.3, J₃=1.6, 1H, CH═CH—H_{cis}); 4.25 (dd, J₁=J₂=8.0, 1H, H-6); 4.18 (tdd, J1=1.6, J₂=12.4, J₃=5.4, 1H, CH₂—CH═CH₂); 3.96 (tdd, J₁=1.4, J₂=12.4, J₃=6.1, 1H, CH₂—CH═CH₂); 3.44 (dd, J₁=J₂=7.91, 1H, H-6); 3.32 (m, 1H, H-5); 2.77 (ddd, J₁=12.7, J₂=J₃=6.8, 1H, H-4); 2.03 (s, 1H, CH₃COO); 1.50 (ddd, J₁=12.7, J₂=J₃=7.7, 1H, H-4); ¹³C NMR (63 MHz, CDCl₃): δ=170.0 (C═O), 150.2 (C1), 134.2 (CH═CH₂), 122.6 (C2), 117.4 (CH═CH₂), 96.8 (C8), 84.1 (C3), 72.6 (C6), 68.0 (CH₂CH═CH₂), 45.0 (C5), 39.0 (C4), 21.1 (CH₃COO); FT-IR (ATR): 2973 (m, C—H), 2932 (m, C—H), 2887(m, C—H), 1732 (s, C═O), 1444 (w), 1426 (w), 1362 (s), 1295 (m, C—O), 1232 (s, C—O), 1152 (s), 1065 (s), 1023 (s), 991 (s), 891 (s), 826 (m), 782 (w); MS (EI, 70 eV): m/z (%)=225 (2) [M+H]⁺, 182 (5), 167 (42), 150 (8), 142 (13), 134 (27), 124 (32), 111 (25), 95 (52), 94 (100), 93 (26), 83 (18), 79 (38), 67 (45), 66 (74), 57 (12), 55 (42), 53 (18).

19. General Protocol for the Pd-catalyzed Introduction of Pyrimidine Nucleobases

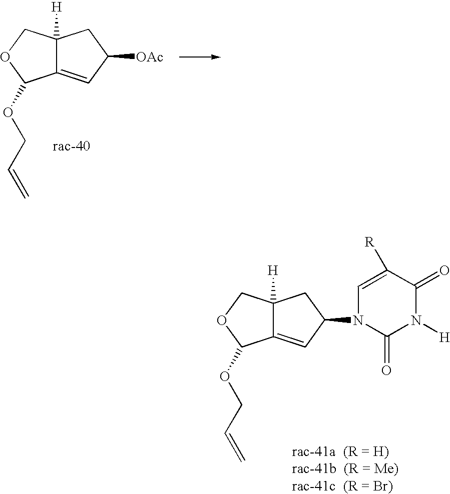

A stirred suspension of a pyrimidine nucleobase (1.65 mmol) and NaH (60 mg, ˜60% dispersion in mineral oil) in dry degassed DMSO (10 ml) is heated at 70° C. under argon for 30 min to form an almost clear solution. After cooling to room temperature, Pd(PPh₃)₄ (58 mg, ca. 5 mol %), PPh₃ (29 mg, 11 mol %) and a solution of the allyl acetate rac-40 (224 mg, 1 mmol) in dry THF (2 ml) are added. Subsequently, the solution is heated at 70° C. under argon for 15 h. The mixture, after having cooled down to room temperature, is then filtered through some silica gel and diluted with CH₂Cl₂ (20 ml). After washing with sat. aqueous NaCl (4×20 ml), drying is performed with MgSO₄, and the solvent is removed under vacuum. The raw product is purified by flash chromatography (EtO-Ac/CyHex=1:9 to 2:1).

20. Preparation of (1RS,3aRS,5RS)-1-(1-allyloxy-3,3a,4,5-tetrahydro-1H-cyclopenta[c]furan-5-yl)-1H-pyrimidine-2,4-dione (rac-41a).

An experiment on a 1 mmol scale yielded rac-41a as a white-yellow solid (188 mg, 66%). The substance contained little impurities (<30%) in the form of Ph₃P═O.

¹H NMR (250 MHz, CDCl₃): δ=10.09 (ψs, 1H, NH); 7.17 (d, J=8.0, 1H, H-6′); 6.04 (dd, J₁=7.2, J₂=8.7, 1H, H-3); 5.86 (tdd, J₁=5.4, J₁=17.2, J₁H, CH═CH₂); 5.70 (d, J=8.0, 1H, H-5′); 5.57 (m, 1H, H-2); 5.43 (s, 1H, H-8); 5.23 (tdd, J₁=1.2, J₂=17.2, J₃=1.6, 1H, CH═CH—Hₜᵣₐₙₛ); 5.13 (tdd, J₁=1.1, J₂=10.3, J₃=1.6, 1H, CH═CH—H_{cis}); 4.24 (dd, J₁=J₂=7.4, 1H, H-6); 4.05 (tdd, J₁=1.6, J₂=12.7, J₁=5.4, 1H, CH₂—CH═CH₂); 4.04 (tdd, J₁=1.3, J₂=12.6, J₃=6.1, 1H, CH₂—CH═CH₂); 3.47 (dd, J₁=J₂=7.6, 1H, H-6); 3.44 (m, 1H, H-5); 2.84 (ddd, J₁=12.5, J₂=7.0, J₁=6.8, 1H, H-4); 1.37 (ddd, J₁=12.5, J₂=8.3, J₃=7.6, 1H, H-4); ¹³C NMR (63 MHz, CDCl₃): δ=163.5 (C4′), 152.4 (C1), 150.8 (C2′), 140.4 (C6′), 133.8 (CH═CH₂), 120.6 (C2), 117.5 (CH═CH₂), 103.0 (C5′), 96.2 (C8), 71.8 (C6), 67.9 (CH₂CH═CH₂), 65.5 (C3), 45.4 (C5), 40.4 (C4); FT-IR (ATR): 3180 (w, N—H), 3051 (w, N—H), 2970 (w, C—H), 2886 (w, C—H), 1681 (m, C═O), 1626 (w, C═C), 1456 (m), 1375 (m), 1296 (w), 1242 (m, C—O), 1060 (m), 989 (m), 812 (w), 760 (w); MS (EI, 70 eV): m/z (%): 277 (8) [M+1]⁺, 276 (5) [M]⁺, 247 (12), 235 (9), 219 (78), 205 (100), 189 (6), 177 (7), 164 (13), 162 (38), 134 (52), 119 (37), 113 (23), 91 (13), 79 (57), 67 (15), 53 (11).

21. Preparation of (1RS,3aRS,5RS)-1-(1-allyloxy-3,3a,4,5-tetrahydro-1H-cyclopenta(c]furan-5-yl)-5-methyl-1H-pyrimidine-2,4-dione (rac-41b).

An experiment on a 2 mmol scale yielded rac-41b in 63% yield.

Melting point=164-166° C. (EtOAc/Hex); ¹H NMR (250 MHz, CDCl₃): δ=8.63 (ψs, 1H, NH); 6.96 (dq, J1=1.3, J₂=1.0, 1H, H-6′); 6.04 (dddd, J₁=2.2, J₂=1.3, J₃=6.8, J₄=8.5, 1H, H-3); 5.91 (tdd, J₁=5.4, J₂=17.2, J₃=10.3, 1H, CH═CH₂); 5.60 (m, 1H, H-2); 5.50 (s, 1H, H-8); 5.29 (tdd, J₁=1.2, J₂=17.2, J₁=1.6, 1H, CH═CH—Hₜᵣₐₙₛ); 5.20 (tdd, J₁=1.3, J₂=10.3, J₃=1.7, 1H, CH═CH—H_{cis}); 4.30 (dd, J₁=J₂=8.1, 1H, H-6); 4.20 (tdd, J₁=1.6, J₂=12.6, J₃=5.4, 1H, CH₂CH═CH₂); 4.04 (tdd, J₁=1.3, J₂=12.6, J₃=6.1, 1H, CH₂—CH═CH₂); 3.53 (dd, J₁=J₂=7.8, 1H, H-6); 3.45 (m, 1H, H-5); 2.86 (ddd, J₁=12.4, J₂=7.0, J₃=6.8, 1H, H-4); 1.91 (d, J=1.0, 1H, CH₃); 1.42 (ddd, J₁=12.4, J₂=J₃=7.7, 1H, H-4); ¹³C NMR (63 MHz, CDCl₃): δ=163.4 (C4′), 152.3 (C1), 150.5 (C2′), 136.1 (C6′), 134.0 (CH═CH2), 120.9 (C2), 117.7 (CH═CH₂), 111.7 (C5′), 96.4 (C8), 72.01 (C6), 68.1 (CH₂CH═CH₂), 65.4 (C3), 45.5 (C5), 40.5 (C4) 12.5 (CH₃); FT-IR (ATR): 3171 (w, N—H), 3052(w, N—H), 2923 (w, C—H), 2887 (w, C—H), 1682 (s, C═O), 1467 (m), 1364 (m), 1297 (m, C—O), 1246 (m, C—O), 1061 (m), 989 (m), 825 (m), 728 (w); MS (EI, 70 eV): m/z (%): 249 (1), 221 (2), 207 (20), 191 (5), 179 (4), 165 (7), 153 (8), 141 (6), 133 (10), 126 (17), 111 (14), 105 (18), 98 (21), 97 (42), 91 (23), 85 (17), 83 (35), 71 (23), 70 (28), 69 (48), 57 (62), 55 (100).

22. Preparation of (1RS,3aRS,5RS)-1-(1-allyloxy-3,3a,4,5-tetrahydro-1H-cyclopenta[c]furan-5-yl)-5-bromo-1H-pyrimidine-2,4-dione (rac-41c).

An experiment on a 2 mmol scale yielded rac-41c in 83% yield.

Melting point 156-158° C.; ¹H NMR (250 MHz, CDCl₃): δ=8.81 (ψs, 1H, NH); 7.47 (s, 1H, H-6′); 6.03 (m, 1H, H-3); 5.89 (tdd, J₁=5.4, J₂=17.1, J₃=10.2, 1H, CH═CH₂); 5.60 (m, 1H, H-2); 5.51 (s, 1H, H-8); 5.28 (tdd, J₁=1.2, J₂=17.1, J₃=1.5, 1H, CH═CH—Hₜᵣₐₙₛ); 5.18 (tdd, J₁=1.2, J₂=10.2, J₂=1.5, 1H, CH═CH—H_{cis}); 4.29 (dd, J₁=J₂=7.8, 1H, H-6); 4.21 (tdd, J₁=1.5, J₂=12.7, J₃=5.4, 1H, CH₂—CH═CH₂); 4.18 (tdd, J₁=1.3, J₂=12.7, J₃=5.4, 1H, CH₂—CH═CH₂); 3.54 (dd, J₁=7.8, J₂=7.6, 1H, H-6); 3.45 (m, 1H, H-5); 2.90 (ddd, J₁=12.4, J₂=J₃=6.8, 1H, H-4); 1.42 (ddd, J₂=12.4, J₂=8.3, J₃=8.8, 1H, H-4); ¹³C NMR (63 MHz, CDCl₃): δ=159.0 (C4′), 152.6 (C1), 150.0 (C2′), 140.0 (C6′), 133.7 (CH═CH₂), 120.2 (C2), 117.3 (CH═CH₂), 97.1 (C5′), 96.1 (C8), 71.6 (C6), 67.7 (CH₂CH═CH₂), 65.8 (C3), 45.3 (C5), 40.4 (C4); FT-IR (ATR): 3167 (w, N—H), 3046 (w, N—H), 2975 (w, C—H), 2885 (w, C—H), 2829 (w, CH), 1691(s, C═O), 1615 (m, C═C), 1441 (m), 1338 (w), 1296 (w, C—O), 1242 (m, C—O), 1061 (w), 990 (m), 820 (w), 750 (m); MS (EI, 70 eV): m/z (%): 356(4) [⁸¹BrM]⁺, 354 (4) [⁷⁹BrM]⁺, 327 (5), 325 (6), 299 (40), 297 (41), 285 (13), 283 (12), 277 (12), 254 (7), 242 (9), 240 (12), 228 (4), 226 (7), 214 (10), 212 (10), 193 (13), 191 (14), 165 (11), 149 (5), 147 (8), 135 (55), 119 (51), 107 (29), 95 (14), 93 (21), 91 (23), 81 (31), 79 (100), 77 (34), 67 (17), 66 (18), 65 (18), 57 (4), 55 (8), 53 (13); HRMS (EI) C₁₄H₁₅BrN₂O₅: calc. 354.022 [⁷⁹BrM]⁺; found: 354.021.

23. Preparation of (1RS,3aRS,5RS)-9-(1-allyloxy-3,3a,4,5-tetrahydro-1H-cyclopenta[c]furan-5-yl)-9H-purine-6-ylamine (rac-41d).

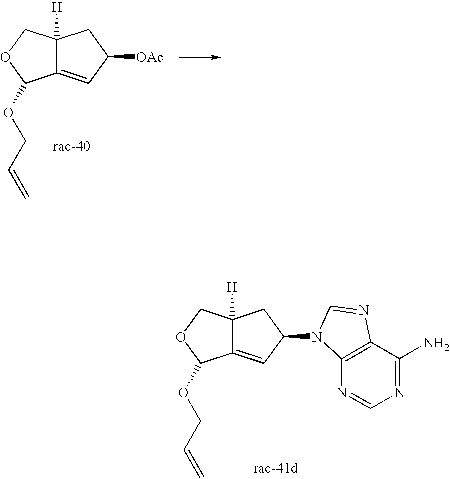

A suspension of adenine (1.022 g, 7.5 mmol) and anhydrous Cs₂CO₃ (2.47 g, 7.5 mmol) in dry degassed DMSO (25 ml) was stirred under argon at 50° C. for 45 min. After cooling to room temperature, a solution of rac-40 (1.12 g, 5 mmol) in DMSO (5 ml) as well as. Ph₃P (145 mg, 11 mol %) and Pd(PPh₃)₄ (290 mg, 5 mole %) were added, and the mixture obtained was stirred at 50° C. for 15 h. After cooling to room temperature, the reaction mixture was filtered with CH₂Cl₂ through some silica gel. The filtrate was washed with sat. NaCl solution (4×100 ml), dried with MgSO₄ and concentrated under vacuum. The residue was purified by flash chromatography (EtOAc/MeOH=9+1) to obtain pure rac-41d as a yellow wax in 73% yield.

¹H NMR (250 MHz, CDCl₃): δ=8.29 (s, 1H, H-2′); 7.47 (s, 1H, H-8′); 6.42 (ψs, 2H, NH₂); 6.04 (tdd, J₁=2.0, J₂32 7.3, J₃=8.6, 1H, H-3); 5.90 (dddd, J₁=5.4, J₂=6.1, J₃=17.1, J₄=10.2, 1H, CH═CH₂); 5.83 (m, 1H, H-2); 5.51 (s, 1H, H-8); 5.26 (tdd, J₁=1.4, J₂=17.1, J₃=1.8, 1H, CH═CH—Hₜᵣₐₙₛ); 5.17 (tdd, J₁=1.2, J₂=10.2, J₃=1.8, 1H, CH═CH—H_{cis}); 4.29 (dd, J₁=J₂=7.1, 1H, H-6); 4.19 (tdd, J₁=1.5, J₂=12.6, J₃=5.4, 1H, CH₂—CH═CH₂); 4.03 (tdd, J₁=1.2, J₂=12.6, J₃=6.1, 1H, CH₂—CH═CH₂); 3.56 (dd, J₁=7.1, J₂=7.5, 1H, H-6); 3.51 (m, 1H, H-5); 2.99 (ddd, J₁=12.4, J₂=7.1, J₃=6.6, 1H, H-4); 1.75 (ddd, J₁=12.4, J₂=8.6, J₃=9.1, 1H, H-4); ¹³C NMR (63 MHz, CDCl₃): δ=155.8 (C6′), 152.9 (C2′), 151.9 (C1), 149.5 (C4′), 138.2 (C8′), 133.9 (CH═CH₂), 121.3 (C2), 119.6 (C5′), 117.6 (CH═CH₂), 96.5 (C8), 72.0. (C6), 68.1 (CH₂CH═CH₂), 64.1 (C3), 45.9 (C5), 41.9 (C4); FT-IR (ATR): 3315 (m, N—H), 3162 (m, N—H), 2970 (w, C—H), 2918 (w, C—H), 2887 (w, C—H), 1644 (s, C═C), 1596 (s), 1571 (m), 1471 (m), 1413 (m), 1327 (m), 1299 (m), 1247 (m, C—O), 1061 (m), 989 (m), 831 (w), 797 (w), 722 (m); MS (EI, 70 eV): m/z (%): 299 (3) [M]⁺, 258 (61), 242 (57), 228 (9), 212 (16), 200 (15), 164 (63), 136 (100), 135 (63), 123 (17), 108 (21), 95 (18), 79 (39), 67 (18), 55 (18), HRMS (EI) C₁₅H₁₇N₅O₂: calc. 299.138 [M]⁺, found: 299.139.

25. General Protocol for the Hydrolysis and Protection of the Products of Type 41′.

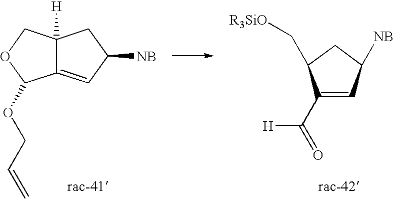

A solution of rac-41′ (1 mmol) and PyH⁺TsO⁻ (76 mg, 0.3 mmol) in wet acetone (10 ml) is heated to reflux for 3 h. Then, the solvent is removed under vacuum, and the flask is flushed with argon. The residue is dissolved in dry pyridine (3 ml), and a chlorotrialkylsilane (1.5 mmol) is added. After stirring for 15 h at room temperature, the solution is partitioned between sat. NaHCO₃ (aq.) (20 ml) and EtOAc (10 ml) (stirring at room temperature for 30 min). Then, the water phase is extracted with EtOAc (2×30 ml), and the combined organic phases are washed with sat. aqueous solutions of NaHCO₃ (40 ml) and NaCl (40 ml). After drying (MgSO₄), the solution is concentrated under vacuum, and the raw product (rac-42′) is purified by flash chromatography.

26. Preparation of (3RS,5RS)-5-[dimethyl-(1,1,2-trimethylpropyl)silanyloxymethyl]-3-(2,4-dioxo-3,4-dihydro-2H-pyrimidin-1-yl)cyclopent-1-enecarbaldehyde (rac-42a).

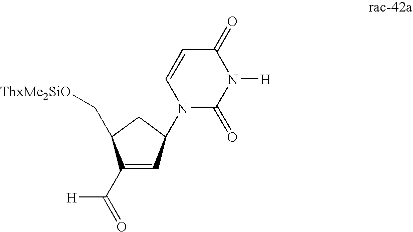

An experiment on a 0.5 mmol scale yielded rac-42a in 80% yield as a yellowish-white solid, starting from rac-41a and thexyldimethylsilyl chloride (according to protocol 25).

Melting point: 191° C. (dec.); ¹H NMR (250 MHz, CDCl₃): δ=9.85 (s, 1H, HC═O), 8.78 (ψs, 1H, NH), 7.47 (d, J₁=8.0, 1H, H-6′), 5.56 (dd, J₁=J₂=2.2, 1H, H-2), 5.90 (dddd, J₁=J₂=2.2, J₃=9.5, J₄=7.1, 1H, H-3), 5.71 (dd, J₁=8.0, J₂=2.4, 1H, H-5′), 4.27 (dd, J₁=10.0, J₂=3.0, 1H, SiOCHₐ), 3.58 (dd, J₁=10.0, J₂=2.5, 1H, SiOCH_{b}), 3.18 (m, 1H, H-5), 2.79 (ddd, J₁=14.2, J₂=J₃=9.5, 1H, H-4), 1.82 (ddd, J₁=14.2, J₂=J₃=7.5, 1H, H-4), 1.56 (septet, J=6.8, 1H, Me₂CH), 0.81 (d, J=6.8 Hz, 6H, (CH₃)₂CH), 0.788+0.786 (ψs, 6H, C(CH₃)₂), 0.042+0.036 (ψs, 6H, SiCH₃); ¹³C NMR (63 MHz, CDCl₃): δ=188.9 (CH═O), 163.2 (C4′), 150.8 (C2′), 150.3 (C1), 147.8 (C2), 141.4 (C6′), 103.2 (C5′), 62.2 (CH₂OSi); 58.9 (C3), 44.3 (C5), 34.0 Me₂CH), 33.3 (C4), 25.4 (Me₂CSi), 20.3 and 20.2 ((CH₃)₂CH), 18.5 and 18.4 ((CH₃)₂CSi), −3.5 and −3.6 ((CH₃)₂Si); FT-IR (ATR): 3038 (w, N—H), 2955 (m, C—H), 2865 (w, C—H), 1697 (s, C═O), 1680 (s, C═O), 1649 (s, C═C), 1473 (w), 1445 (w), 1425 (w), 1383 (m), 1276 (m), 1248 (m, C—O), 1186 (m), 1111 (m), 1063 (w), 1019 (w), 998 (w), 979 (w), 849 (m), 828 (m), 804 (m), 779 (m), 761 (m); MS (EI, 70 eV): m/z (%): 295 (7), 294 (17), 293 (100) [M−85]⁺, 275 (27), 250 (18), 201 (55), 181 (62), 169 (26), 151 (8), 137 (5), 99 (7), 91 (5), 89 (7), 85 (6), 77 (6), 75 (23), 73 (18), 69 (6), 59 (8), 57 (5); HRMS (EI) C₁₃H₁₇N₂O₄Si [M−85]⁺: calc. 293.096, found: 293.096.

27. Preparation of (3RS,5RS)-5-[dimethyl-(1,1,2-trimethylpropyl)-silanyloxymethyl]-3-(5-methyl-2,4-dioxo-3,4-dihydro-2H-pyrimidin-1-yl)cyclopent-1-enecarbaldehyde (rac-42b).

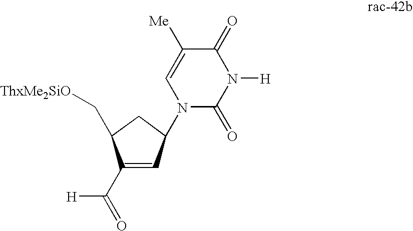

An experiment on a 0.7 mmol scale yielded rac-42b in 65% yield as a yellowish solid, starting from rac-41b and thexyldimethylsilyl chloride (according to protocol 25).

Melting point: 187° C.; ¹H NMR (250 MHz, CDCl₃): δ=10.06 (ψs, 1H, NH), 9.81 (s, 1H, HC═O), 6.99 (q, J=1.2, 1H, H-6′), 6.58 (m, 1H, H-2), 5.83 (dddd, J₁=J₂=2.4, J₃=J₄=8.6,1H, H-3), 4.18 (dd, J₁=10.0, J₂=3.4, 1H, SiOCHₐ), 3.53 (dd, J₁=10.0, J₂=2.4, 1H, SiOCH_{b}), 3.10 (m, 1H, H-5), 2.65 (ddd, J₁=13.4, J₂=J₃=8.8, 1H, H-4), 1.85 (d, J=1.2, 3H, CH₃), 1.80 (ddd, J₁=13.4, J₂=J₃=8.4, 1H, H-4), 1.51 (Sept., J=7.1, 1H, Me₂CH), 0.83 (d, J=7.1, 6H, (CH₃)₂CH], 0.793+0.790 (ψs, 6H, C(CH₃)₂), 0.058+0.057 (ψs, 6H, SiCH₃); ¹³C NMR (63 MHz, CDCl₃): δ=188.9 (CH═O), 164.1 (C4′), 151.1 (C2′), 149.8 (C1), 148.4 (C2), 136.2 (C6′), 111.8 (C5′), 61.4 (CH₂OSi), 59.0 (C3), 44.2 (C5), 34.0 (Me₂CH), 33.4 (C4), 25.1 (Me₂CSi), 20.2 and 20.2 ((CH₃)2CH), 18.4 and 18.4 ((CH₃)₂CSi), 12.3 (CH₃), −3.5 and −3.7 (SiCH₃); FT-IR (ATR): 3177 (w, N—H), 3050 (w, N—H), 2954 (m, C—H), 2864 (m, C—H), 1683 (s, C═O), 1464 (m), 1376 (m), 1279 (m), 1249 (m, C—O), 1153 (w), 1113 (m), 1057 (w), 1017 (w), 982 (w), 874 (m), 830 (m), 778(m), 722 (w); MS (EI, 70 eV): m/z (%): 393 (4) [M+1]⁺, 365 (13), 341 (5), 307 (100), 289 (27), 264 (10), 215 (52), 183 (31), 181 (73), 167 (12), 151 (7), 137 (5), 113 (4), 89 (7), 75 (15), 59 (4); HRMS (ESI) C₂₀H₃₃N₂O₄Si [M+1]⁺: calc. 393.2209, found 393.2209.

28. Preparation of (3RS,5RS)-3-(5-bromo-2,4-dioxo-3,4-dihydro-2H-pyrimidin-1-yl)-5-[dimethyl-(1,1,2-trimethylpropyl)silanyloxymethyl]-cyclopent-1-enecarbaldehyde (rac-42c).

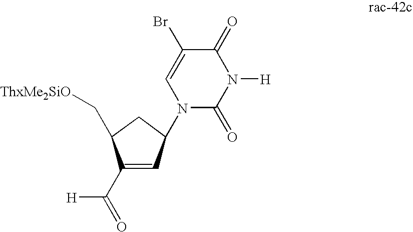

An experiment on a 1 mmol scale yielded rac-42c in 83% yield as a colorless solid, starting from rac-41c and thexyldimethylsilyl chloride (according to protocol 25).

Melting point: 163° C. (dec.); ¹H NMR (250 MHz, CDCl₃): δ=9.87 (s, 1H, HC═O), 8.52 (ψs, 1H, NH), 7.54 (s, 1H, H-6′), 6.57 (m, 1H, H-2), 5.85 (dddd, J₁=J₂=2.3, J₃=8.7, J₄=7.6, 1H, H-3), 4.25 (dd, J₁=10.0, J₂=3.2, 1H, SiOCHₐ), 3.52 (dd, J₁=10.0, J₂=2.3, 1H, SiOCH_{b}), 3.17 (m, 1H, H-5), 2.75 (ddd, J₁=13.7, J₂=J₃=8.8, 1H, H-4), 1.85 (ddd, J₁=13.7, J₂=J₃=7.7, 1H, H-4), 1.55 (septet, J=6.8, 1H, Me₂CH), 0.83 (d, J=6.8, 6H, (CH₃)₂CH), 0.810+0.805 (ψs, 6H, C(CH₃)₂), 0.06 (s, 6H, SiCH₃), ¹³C NMR (63 MHz, CDCl₃): δ=188.9 (CH═O), 159.2 (C4′), 150.5 (C2′), 150.4 (C1), 147.3 (C2), 140.2 (C6′), 97.7 (C5′), 61.5 (CH₂OSi), 59.9 (C3), 44.3 (C5), 34.0 (Me₂CH), 33.5 (C4), 25.3 (Me₂CSi), 20.4 and 20.3 ((CH₃)₂CH), 18.5 and 18.4 ((CH₃)₂CSi), −3.5 and −3.7 (CH₃Si); FT-IR (ATR): 3177 (w, N—H), 3046 (w, N—H), 2954 (m, C—H), 2862 (w, C—H), 2823 (w), 1704 (s, C═O), 1684 (s, C═O), 1617 (m, C═C), 1444 (m), 1368 (w), 1276 (m), 1249 (m, C—O), 1150 (w), 1109 (m), 1038 (w), 982 (w), 847 (m), 830 (m); MS (EI, 70 eV): m/z (%)=374 (22), 373 (95), 372 (28), 371 (100) [M−85]⁺, 355 (34), 353 (32), 339 (7), 328 (13), 297 (3), 281 (92), 279 (89), 251 (10), 249 (47), 226 (3), 224 (5), 184 (3), 183 (11), 182 (58), 181 (90), 167 (28), 152 (3), 151 (12), 137 (10), 116 (5), 91 (7), 89 (14), 85 (10), 75 (31), 73 (18), 59 (8); HRMS (EI) C₁₃H₁₆BrN₂O₄Si [M−85]⁺: calc. 371.006, found 371.005.

29. Preparation of (3RS,5RS)-3-(5-bromo-2,4-dioxo-3,4-dihydro-2H-pyrimidin-1-yl)-5-[tert-butyidiphenyisilanyloxymethyl]cyclopent-1-ene-carbaldehyde (rac-42d).

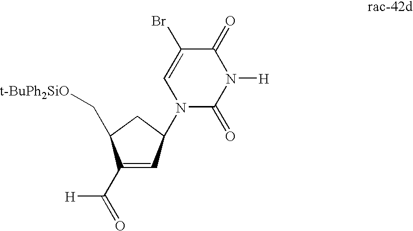

An experiment on a 1 mmol scale yielded rac-42d in 76% yield as a colorless solid, starting from rac-41c and tert.-butyldiphenylsilyl chloride (according to protocol 25).

Melting point: 164° C.; ¹H NMR (250 MHz, CDCl₃): δ=10.24 (ψs, 1H, NH), 9.86 (s, 1H, HC═O), 7.75-7.32 (m, 10H, 2×Ph), 7.48 (s, 1H, H-6′), 6.65 (dd, J₁=J₂=2.0, 1H, H-2), 5.80 (dddd, J₁=J₂=2.1, J₃=J₄=8.6, 1H, H-3), 4.21 (dd, J₁=10.3, J₂=4.4, 1H, SiOCHₐ), 3.52 (dd, J₁=10.3, J2=2.7, 1H, SiOCH_{b}), 3.15 (m, 1H, H-5), 2.72 (ddd, J₁=13.7, J₂J₃=8.6, 1H, H-4ₐ), 1.85 (ddd, J₁=13.7, J₂=J₃=8.4, 1H, H-4_{b}), 1.07+1.06 (ψs, 9H, (CH₃)₃C); ¹³C NMR (250 MHz, CDCl₃) δ=188.8 (CH═O), 159.3 (C4′), 150.4 (C2′), 150.4 (C1), 147.1 (C2), 140.0 (C6′), (136.0+135.5) and (133.0+132.8) and (129.8+129.4) and (127.7+127.6) (all of Ph), 97.8 (5′), 62.5 (CH₂OSi), 60.1 (C3), 44.3 (C5), 34.0 (C4), 26.9 and 26.8 and 26.5 ((CH₃)₃CSi), 19.3 ((CH₃)₃CSi); FT-IR (cm⁻¹, ATR): 3177 (w, N—H), 3067 (w, N—H), 2928 (m, C—H), 2854 (m, C—H), 1707 (s, C═O), 1683 (s, C═O), 1619 (m, C═C), 1588 (w), 1469 (w), 1443 (m), 1426 (m), 1362 (w), 1276 (m), 1261 (m), 1242 (m,C—O), 1151 (w), 1110 (m), 1060 (w), 1037 (w), 997 (w), 983 (w), 839 (m), 821 (m), 741 (m), 701 (s).

30. Preparation of (3RS,5RS)-3-(6-aminopurin-9-yl)-5-[dimethyl-(1,1,2-trimethylpropyl)silanyloxymethyl]cyclopent-1-enecarbaldehyde (rac-42e).

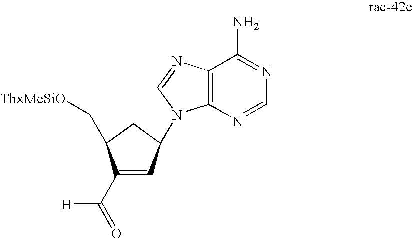

An experiment on a 1 mmol scale yielded rac-42e in 45% yield as a yellow-orange solid, starting from rac-41d and thexyldimethylsilyl chloride (according to protocol 25, but using 2 equiv. of PyH+TsO (1st step) and 3.3 equiv. of thexMe₂SiCl, 3.3 equiv. of Et₃N, 0.3 equiv. of DMAP in 5 ml of CH₂Cl₂ (2nd step).

Melting point: 144° C. (dec.); ¹H NMR (250 MHz, CDCl₃): δ=9.86 (s, 1H, HC═O), 8.30 (s, 1H, H-2′), 7.93 (s, 1H, H-8′), 6.75 (dd, J₁=J₂=2.2, 1H, H-2), 6.40 (s, 2H, NH₂), 5.84 (dddd, J₁=J₂=2.2, J₃=9.1, J₄=6.8, 1H, H-3), 4.14 (dd, J₁=10.0, J₂=3.8, 1H, SiOCHₐ), 3.66 (dd, J₁=10.0, J₂=2.4, 1H, SiOCH_{b}), 3.24 (m, 1H, H-5), 2.89 (ddd, J₁=14.0, J₂=J₃=9.1, 1H, H-4), 2.10 (ddd, J₁=14.0, J₂=J₃=6.8, 1H, H-4), 1.53 (septet, J=6.8, 1H, Me₂CH), 0.78 (d, J=6.8, 6H, (CH₃)₂CH), 0.77 (s, 6H, C(CH₃)₂), 0.02 (s, 6H, CH₃Si), ¹³C NMR (63 MHz, CDCl₃): δ=189.2 (CH═O), 155.7 (C4′), 152.8 (C2′), 149.5 (C1), 149.5 (C6′), 147.4 (C2), 138.5 (C8′), 119.2 (C5′), 62.2 (CH₂OSi), 57.5 (C3), 44.7 (C5), 35.1 (C4); 34.0 (Me₂CH), 25.3 (Me₂CSi), 20.3 and 20.2 ((CH₃)₂CH), 18.4 and 18.4 ((CH₃)₂CSi), −3.6 (CH₃Si); FT-IR (ATR):. 3318 (w, N—H), 3168 (w, N—H), 2953 (m, C—H), 2864 (w, C—H), 1683 (s, C═O), 1645 (s, C═C), 1598 (s), 1575 (m), 1468 (m), 1413 (m), 1327 (m), 1297 (m), 1250 (s, C—O), 1149 (w), 1109 (m), 1085 (m), 1085 (m), 1015 (w), 979 (w), 830 (m), 777 (m), 649. (m); MS (EI, 70 eV): m/z (%): 401(2) [M]⁺, 318 (8), 317 (24), 316 (100), 302 (7), 242 (4), 224 (5), 212 (4), 192 (5), 182 (11), 181 (30), 167 (8), 151 (7), 137 (4), 136 (12), 135 (10), 108 (3), 85 (4), 75 (17), 59 (4); HRMS (ESI) C₂₀H₃₁N₅O₂Si: calc. 402.2325, found 402.2327.

31. General Protocol for the Conversion of Aldehydes of Type 42′ into Esters of Type 43′ by Wittig Reaction.

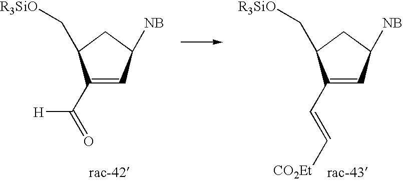

To a solution of the required Wittig ylide (202 mg, 1.1 eq.) in dry THF (2 ml), a solution of the aldehyde rac-42′ (500 μmol, 1 eq.) in dry THF (4 ml) is added at 50° C. After stirring at 50° C. for 16 h, the reaction mixture is cooled down to room temperature, slightly concentrated under vacuum, and the product rac-42′ is purified by chromatography on silica gel (EtOAc/CyHex=1+2).

32. Preparation of (3RS,5RS)-3-[5-[dimethyl-(1,1,2-trimethylpropyl)-silanyloxymethyl]-3-(2,4-dioxo-3,4-dihydro-2H-pyrimidine-1-yl)cyclo-pent-1-enyl]acrylic acid ethyl ester (rac-43a).

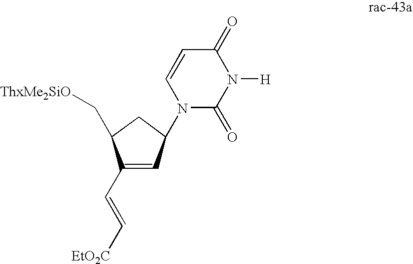

An experiment on a 0.25 mmol scale yielded rac-43a in 96% yield as a pale-yellow solid, starting from rac-42a (according to protocol 31).

Melting point 92-96° C. (EtOAc/Hex); ¹H NMR (250 MHz, CDCl₃): δ=8.45 (s, 1H, NH), 7.47 (d, J_{cis}=8.0, 1H, H-5″), 7.38 (d, Jₜᵣₐₙₛ=16.2, 1H, H-2), 6.06 (d, Jₜᵣₐₙₛ=16.2, 1H, H-3), 5.91 (s, 1H, H-2′), 5.71 (m, 1H, H-3′), 5.66 (d, J_{cis}=8.0, 1H, H-6″), 4.22 (q, J=7.2, 2H, OCH₂CH₃), 3.96 (dd, J₁=10.4, J₂=3.8, 1H, SiOCHₐ), 3.63 (dd, J₁=10.5, J₂=2.7, 1H, SiOCH_{b}), 3.08 (m, 1H, H-5′), 2.79 (ddd, J₁=14.3, J₂=J₃=9.5, 1H, H-4′), 1.73 (ddd, J₁=14.5, J₂=J₃=5.5, 1H, H-4′), 1.55 (septet, J_{=6.8, 1}H, Me₂CH), 1.29 (t, J=7.1, 3H, OCH₂CH₃), 0.81 (d, J=6.8, 6H, (CH₃)₂CH), 0.79 (s, 6H; C(CH₃)₂), 0.04 (s, 6H, CH₃Si); ¹³C NMR (63 MHz, CDCl₃): δ=166.4 (C1), 163.0 (C4″), 150.8 (C2″), 146.7 (C1′), 141.7 (C6″), 137.9 (C3), 134.7 (C2′), 121.7 (C2), 102.5 (C5″), 63.2 (CH₂OSi), 60.7 OCH₂CH₃), 59.4 (C3′), 45.8 (C5′), 34.0 (Me₂CH), 33.7 (C4′), 25.4 (Me₂CSi), 20.4 and 20.2 ((CH₃)₂CH), 18.5 and 18.3 ((CH₃)₂CSi), 14.3 (OCH₂CH₃), −3.5 (CH₃Si); FT-IR (ATR): 3184 (w, N—H), 3051(w, N—H), 2955 (m, C—H), 2865 (w, C—H), 1703 (s, C═O), 1687 (s, C═O), 1638 (m, C═C), 1605 (w), 1461 (m), 1378 (m), 1305 (m), 1265 (s, C—O), 1249 (s, C—O), 1174 (s), 1111 (m), 1094 (m), 1064 (w), 1034 (w), 986 (w), 873 (w), 830 (s), 778 (m); MS (EI, 70 eV): m/z (%): 449 (1) [M+1]⁺, 403 (45), 379 (13), 363 (15) [M−85 ]⁺, 253 (8), 252 (25), 251 (100), 205 (37), 188 (7), 187 (41), 177 (33), 169 (58), 149 (9), 133 (5), 132 (8), 131 (48), 104 (13), 103 (44), 99 (18), 89 (17), 77 (7), 75 (35), 74 (9), 73 (38), 59(10), 58 (21); HRMS (EI) C₁₇H₂₃N₂O₅Si (M⁺−85): calc. 363.138, found 363.138.

33. Preparation of (3RS,5RS)-3-[5-[dimethyl-(1,1,2-trimethylpropyl)-silanyloxymethyl]-3-(5-methyl-2,4-dioxo-3,4-dihydro-2H-pyrimidin-1-yl)cyclopent-1-enyl]acrylic acid ethyl ester (rac-43b).

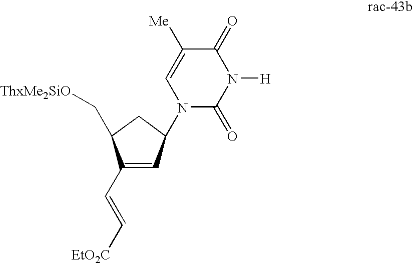

An experiment on a 0.251 mmol scale yielded rac-43b in 95% yield as a pale-yellow solid, starting from rac-42b (according to protocol 31).

Melting point: 132-134° C. (EtOAc/Hex); ¹H NMR (250 MHz, CDCl₃): δ=8.50 (s, 1H, NH), 7.38 (d, Jₜᵣₐₙₛ=16.2, 1H, H-2), 7.05 (s, 1H, H-6″), 6.08 (d, Jₜᵣₐₙₛ=16.2, 1H, H-3), 5.93 (s, 1H, H-2′), 5.65 (m, 1H, H-3′), 4.22 (q, J=7.1, 2H, OCH₂CH₃), 3.87 (dd, J₁=10.4, J₂=4.4, 1H, SiOCHₐ), 3.63 (dd, J₁=10.4, J₂=3.3, 1H, SiOCH_{b}), 3.06 (m, 1H, H-5′), 2.72 (ddd, J₁=13.9, J₂=J₃=8.9, 1H, H-4′), 1.88 (s, 3H, CH-5″), 1.72 (ddd, J₁=13.6, J₂=J₃=6.7, 1H, H-4′), 1.56 (septet, J=6.8, 1H, Me₂CH), 1.29 (t, J=7.1, 3H, OCH₂CH₃), 0.82 (d, J=6.9, 6H, (CH₃)₂CH), 0.79 (s, 6H, C(CH)₂), 0.04 (s, 6H, CH₃Si); ¹³C NMR (63 MHz, CDCl₃): δ=166.4 (C1), 163.6 (C4″), 150.8 (C2″), 146.7 (C1′), 138.1 (C6″), 136.7 (C3), 134.6 (C2′), 121.5 (C2), 111.0 (C5″), 63.0 (CH₂OSi), 60.7 OCH₂CH₃), 59.5 (C3′), 45.9 (C5′), 34.0 (Me₂CH), 33.9 (C4′), 25.3 (Me₂CSi), 20.34 and 20.27 ((CH₃)₂CH), 18.48 and 18.44 ((CH₃)₂CSi), 14.2 (OCH₂CH₃), 12.5 (5″-CH₃), −3.5 (CH₃Si); FT-IR (ATR): 3176 (w, N—H), 3043 (w, N—H), 2954 (m, C—H), 2864 (w, C—H), 1684 (s, C═O), 1639 (s, C═C), 1603 (w), 1464 (m), 1387 (m), 1377 (w), 1364(m), 1305 (m), 1266 (s, C—O), 1249 (s, C-a), 1173 (s), 1159(s), 1112 (m), 1094 (m), 1065 (w), 1034 (w), 985 (w), 873 (w); 830 (s); MS (EI, 70 eV): m/z (%): 449 (1) [M+1]⁺, 403 (45) 379 (13), 363 (15), [M−85]⁺, 253 (8), 252 (25), 251 (100), 205 (37), 188 (7), 187 (41), 177 (33), 169 (58), 149 (9), 133 (5), 132 (8), 131 (48), 104 (13), 103 (44), 99 (18), 89 (17), 77 (7), 75 (35), 74 (9), 73 (38), 59(10), 58 (21); HRMS (EI) C₁₈H₂₅N₂O₅Si (M⁺−85): calc. 377.153, found 377.153.

34. Preparation of (3RS,5RS)-3-[3-(5-bromo-2,4-dioxo-3,4-dihydro-2H-pyrimidin-1-yl)-5-[dimethyl-(1,1,2-trimethylpropyl)silanyloxymethyl]-cyclopent-1-enyl]acrylic acid ethyl ester (rac-43c).

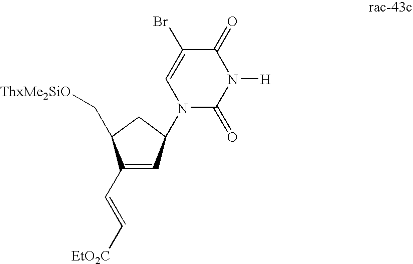

An experiment on a 0.5 mmol scale yielded rac-43c in 82% yield as a colorless solid, starting from rac-42c (according to protocol 31).

Melting point: 164-165° C. (EtOAc/CyHex); ¹H NMR (250 MHz, CDCl₃): δ=10.17 (s, 1H, NH), 7.51 (s, 1H, H-6″), 7.35 (d, Jₜᵣₐₙₛ=16.4, 1H, H-2), 6.04 (d, Jₜᵣₐₙₛ=16.0, 1H, H-3), 5.92 (s, 1H, H-2′), 5.62 (m, 1H, H-3′), 4.17 (q, J=7.1, 2H, OCH₂CH₃), 3.84 (dd, J₁=10.4, J₂=4.2, 1H, SiOCHₐ), 3.57 (dd, J=10.2, J₂=3.2, 1H, SiOCH_{b}), 3.03 (m, 1H, H-5′), 2.72 (ddd, J₁=14.1, J₂=J₃=9.0, 1H, H-4′), 1.68 (ddd, J₁=13.9, J₂=J₃=6.4, 1H, H-4′), 1.51 (sept., J=6.8, 1H, Me₂CH), 1.24 (t, J=7.0, 3H, OCH₂CH₃), 0.77 (d, J=6.8, 6H, (CH₃)₂CH), 0.74 (s, 6H, C(CH₃)₂), 0.01+−0.01 (2×s, 6H, CHSi); ¹³C NMR (125 MHz, CDCl₃): δ=166.2 (C1), 159.3 (C4″), 150.6 (C2″), 147.3 (C1′), 140.3 (C6″), 137.8 (C3), 133.7 (C2′), 121.7 (C2), 96.9 (C5″), 62.8 (CH₂OSi), 60.5 OCH₂CH₃), 60.3 (C3′), 45.7 (C5′), 33.9 (Me₂CH), 33.8 (C4′), 25.2 (Me₂CSi), 20.28 and 20.13 ((CH₃)₂CH), 18.35 and 18.26 ((CH₃)₂CSi), 14.1 (OCH2CH₃), −3.56 and −3.64 ((CH₃)₂Si); FT-R (ATR): 3178 (w, N—H), 3053 (w, N—H), 2954 (m, C—H), 2864 (w, C—H), 1705 (s, C═O), 1637 (m, C═C), 1617 (m), 1442 (m), 1389 (w), 1366 (w), 1337(w), 1306 (m), 1265 (m, C—O), 1249 (m, C—O), 1174 (m), 1112 (m), 1066 (w), 1034 (m), 985 (w), 874 (w), 830 (m), 777 (m).

35. Preparation of (3RS,5RS)-3-[3-(5-bromo-2,4-dioxo-3,4-dihydro-2H-pyrimidin-1yl)-5-(tert-butyldiphenylsilanyloxymethyl)cyclopent-1-enyl]acrylic acid ethyl ester (rac-43d).

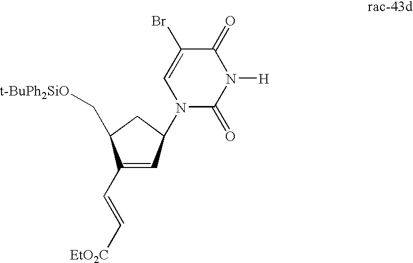

An experiment on a 0.2 mmol scale yielded rac-43d in 72% yield as a colorless oil, starting from rac-42c (according to protocol 31).

¹H NMR (250 MHz, CDCl₃): δ=9.61 (s, 1H, NH), 7.44-7.32 (m, 12H, H-6″, H-3, 2×Ph), 5.94 (d, Jₜᵣₐₙₛ=16.1, 1H, H-3), 5.93 (s, 1H, H-2′), 5.61 (m, 1H, H-3′), 4.22 (q, J=7.1, 2H, OCH₂CH₃), 3.74 (m, 2H, SiOCH₂), 3.08 (m, 1H, H-5′), 2.77 (ddd, J₁=14.2, J₂=J₃=8.8, 1H, H-4′), 1.79 (ddd, J₁=14.1, J₂=J₃=6.3, 1H, H-4′), 1.30 (t, J=7.0, 3H, OCH₂CH₃), 1.04 (s, 9H, C(CH₃)₃); ¹³C NMR (63 MHz, CDCl₃): δ=166.2 (C1), 159.1 (C4″), 150.3 (C2″), 147.7 (C1′), 140.0 (C6″), 137.6 (C3), (135.5+135.4) and (132.9+132.7) and (129.9+129.5) and (127.8+127.7) (all of Ph), 133.4 (C2′), 122.2 (C2), 97.1 (C5″), 64.2 (CH₂OSi), 60.7 (OCH₂CH₃), 60.4 (C3′), 46.0 (C5′), 34.3 (C4′), 26.9 ((CH₃)₃C), 19.2 ((CH₃)₃C), 14.2 (OCH₂CH₃); FT-IR (ATR): 3472 (w), 3178 (w, N—H), 3066 (w, N—H), 2953 (w, C—H), 2854 (w, C—H), 1703 (s, C═O), 1638 (m, C═C), 1617 (m), 1442 (m), 1426 (m), 1367 (w), 1306 (w), 1266 (m, C—O), 1240 (m,C—O), 1175 (m), 1110 (s), 1034 (w), 985 (w), 821 (m), 741 (m), 702 (s).

36. General Protocol for the Fe(CO)₃ Complexing of Dienes of Type 43′.

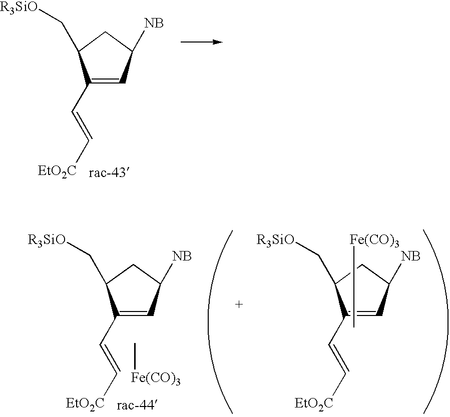

To a stirred suspension of Fe₂(CO)₉ (91 mg, 0.25 mmol) in abs. Et₂O (5 ml), a solution of the diene rac-43′ (0.1 mmol) in abs. Et₂O (1 ml) is added under argon, and the mixture is heated under reflux for 24 h. After cooling down to room temperature, the solvent is removed under vacuum, and the residue is purified by flash chromatography (EtOAc/CyHex=1:4 to =1:2). In addition to the main product rac-44′, some amounts of the diastereomeric complexing product are always obtained as well (separately).

37. Preparation of (3SR,5RS)-3-[5-[dimethyl-(1,1,2-trimethylpropyl)-silanyloxymethyl]-3-(2,4-dioxo-3,4-dihydro-2H-pyrimidin-1-yl)cyclopent-1-enyl]acrylic-acid-ethyl-estertricarbonyliron (rac-44a).

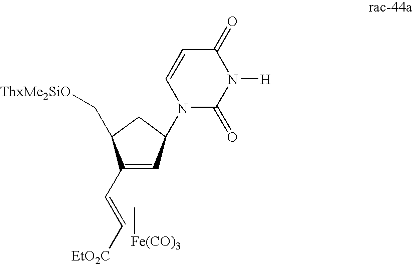

An experiment on a 0.1 mmol scale yielded rac-44a in 37% yield as an orange-red oil, starting from rac-43a (according to protocol 36).

¹H NMR (250 MHz, CDCl₃): δ=8.99 (s, 1H, NH), 7.59 (d, J_{cis}=7.7, 1H, H-5″), 6.06 (d, J=7.9, 1H, H-3), 5.68 (d, J_{cis}=8.0, 1H, H-6″), 5.16 (dd, J₁=10.0, J₂=3.4, 1H, H-3′), 4.10 (m, 2H, OCH₂CH₃), 3.98 (dd, J₁=10.1, J₂=4.9, 1H, SiOCHₐ), 3.78 (dd, J₁=10.2, J₂=5.9, 1H, SiOCH_{b}), 3.14 (m, 1H, H-5′), 2.66 (ddd, J₁=15.5, J₂=J₃=9.5, 1H, H4ₐ′), 1.87 (s, 1H, H-2′), 1.76 (ddd, J₁=15.2, 15.3, J₂=J₃=3.5, 1H, H-4_{b}′), 1.62 (Sept., J=6.8, 1H, Me₂CH), 1.22 (t, J=7.1, 3H, OCH₂CH₃), 0.86 (d, J=6.8, 6H, (CH₃)₂CH), 0.85 (s, 6H, C(CH₃)₂), 0.70 (d, J=7.6, 1H, H-2), 0.14+0.12 (2×s, 6H, CH₃Si); ¹³C NMR (63 MHz, CDCl₃): δ=171.8 (C1), 163.0 (C4″), 150.5 (C2″), 141.2 (C6″), 113.1 (C1′), 102.7 (C5″), 78.2 (C3), 67.3 (CH₂OSi), 64.2 (C2′), 60.7 (OCH₂CH₃), 59.4 (C3′), 49.6 (C2), 45.4 (C5′), 34.0 (Me₂CH), 33.9 (C4′), 25.5 (Me₂CSi), 20.5 and 20.3 ((CH₃)₂CH), 18.5 and 18.4 ((CH₃)₂CSi), 14.1 (OCH₂CH₃), −3.2 and −3.3 (CH₃Si); FT-IR (ATR): 3189 (w, N—H), 3053 (w, N—H), 2956 (m, C—H), 2865 (w, C—H), 2055 (s, C≡O), 1992 (s, C≡O), 1974 (s, C≡O), 1701 and 1697 and 1692 and 1681 (s, C═O), 1630 (m, C═C), 1495 (w), 1461 (m), 1427 (m), 1375 (m), 1275 (m, C—O), 1250 (m, C—O), 1177 (m), 1098 (m), 1033 (m), 873 (w), 830 (m), 778 (m), 615 (m), 607 (m).

38. Preparation of (3RS,5RS)-3-[5-[dimethyl-(1,1,2-trimethylpropyl)-silanyloxymethyl]-3-( 5-methyl-2,4-dioxo-3,4-dihydro-2H-pyrimidin-1-yl)cyclopent-1-enyl]acrylic-acid-ethyl-estertricarbonyliron (rac-44b).

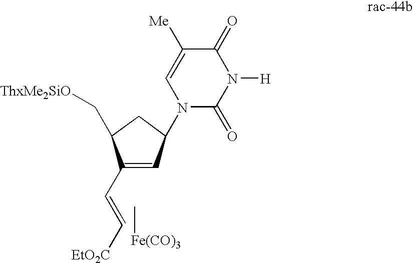

An experiment on a 0.1 mmol scale yielded rac-44b in 40% yield as an orange-red oil, starting from rac-43b (according to protocol 36). ¹H NMR (250 MHz, CDCl₃):δ=8.56 (s, 1H, NH), 7.10 (s, 1H, H-6″), 6.12 (d, J=7.9, 1H, H-3), 5.07 (dd, J_{1=9.4}, J₂=3.6, 1H, H-3′), 4.11 (m, 2H, OCH ₂CH₃), 3.84 (dd, J₁=10.2, J₂=6.3, 1H, SiOCHₐ), 3.73 (dd, J₁=10.1, J₂=7.6, 1H, SiOCH_{b}), 3.12 (m, 1H, H-5′), 2.60 (ddd, J₁=15.5, J₂=J₃ 9.1, 1H, H-4ₐ′), 1.89 (s, 4H, H-2′+CH₃), 1.73-1.56 (m, 2H, H-4_{b}′+Me₂CH), 1.23 (t, J=7.2, 3H, OCH₂CH₃), 0.88 (d, J=6.5, 6H, (CH₃)₂CH), 0.87 (s, 6H, C(CH₃)₂), 0.72 (d, J=7.8, 1H, H-2), 0.13 (s, 6H, CH₃Si); ¹³C NMR (63 MHz, CDCl₃): δ=171.7 (C1), 163.3 (C4″), 150.4 (C2″), 136.3 (C6″), 112.9 (C1′), 111.2 (C5″), 78.9 (C3), 66.9 (CH₂OSi), 63.4 (C2′), 60.6 (OCH₂CH₃), 60.0 (C3′), 46.6 (C2), 45.5 (C5′), 34.1 (Me₂CH), 33.8 (C4′), 25.4 (Me₂CSi), 20.4 and 20.3 ((CH₃)₂CH), 18.6 and 18.5 ((CH₃)₂CSi), 14.1 (OCH₂CH₃), 12.6 (5″-CH₃), −3.3 (CH₃Si); FT-IR (ART): 3178 (w, N—H), 3047 (w, N—H),2955 (m, C—H), 2864 (w, C—H), 2094 (w, C≡O), 2056 (s, C≡O), 2023 (s, C≡O), 1991 (s, C≡O), 1701 and 1698 and 1692 (s, C═O), 1465 (m), 1450 (w), 1426 (w), 1389(w), 1376 (w), 1366 (w), 1278 (m, C—O), 1250 (m, C—O), 1220 (w), 1175 (m), 1111 (m), 1094 (m), 1035 (w), 873 (w), 830 (m), 777 (m), 621 (m), 610 (m).

39. Preparation of (3RS,5RS)-3-[3-(5-bromo-2,4-dioxo-3,4-dihydro-2H-pyrimidin-1-yl)-5-[dimethyl-(1,1,2-trimethylpropyl)silanyloxymethyl]-cyclopent-1-enyl]acrylic-acid-ethyl-estertricarbonyliron (rac-35=rac-44c).

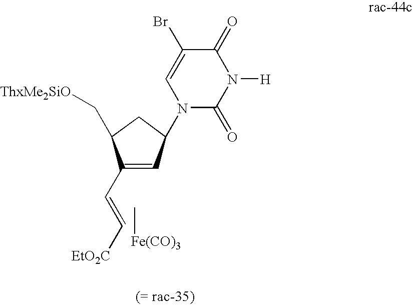

An experiment on a 0.1 mmol scale, starting from rac-43c, yielded rac-44c in 73% yield as a yellow oil which crystallized from cyclohexane (according to protocol 36).

Melting point: 100-104° C. (CyHex); ¹H NMR (250 MHz, CDCl₃): δ=8.79 (s, 1H, NH), 7.64 (s, 1H, H-6″), 6.13 (d, J=7.9, 1H, H-3), 5.09 (dd, J₁=9.6, J ₂=3.6, 1H, H-3′), 4.12 (m, 2H, OCH₂CH₃), 3.87 (dd, J₁=10.1, J₂=6.2, 1H, SiOCHₐ), 3.73 (dd, J₁=10.1, J₂=7.2, 1H, SiOCH_{b}), 3.14 (m, 1H, H-5′), 2.63 (ddd, J₁=15.3, J₂=J₃=9.4, 1H, H-b 4ₐ′), 1.85 (s, 1H, H-2′), 1.72 (ddd, J₁=15.6, J₂=J₃3.9, 1H, H-4_{b}′), 1.65 (Sept., J=6.9, 1H, Me₂CH), 1.23 (t, J=7.1, 3H, OCH₂CH₃), 0.88 (d, J=6.6, 6H, (CH₃)₂CH), 0.88 (s, 6H, C(CH₃)₂), 0.76 (d, J=7.7, 1H, H-2), 0.16 +0.15 (2×s, 6H, CH₃Si); ¹³C NMR(63 MHz, CDCl₃): δ=171.6 (C1), 158.6 (C4″), 149.7 (C2″), 140.0 (C6″), 112.9 (C1′), 97.0 (C5″), 79.0 (C3), 67.0 (CH₂OSi), 62.4 (C2′), 60.73 (OCH₂CH₃), 60.69 (C3′), 46.5 (C2), 45.7 (C5′), 34.1 (Me₂CH), 33.9 (C4′), 25.5 (Me₂CSi), 20.5 and 20.4 ((CH₃)₂CH), 18.6 and 18.5 ((CH₃)₂CSi), 14.1 (OCH₂CH₃), −3.22 and −3.24 ((CH₃)₂Si); FT-R (ATR): 3179 (w, N—H), 3055 (w, N—H), 2954 (m, C—H), 2926 (m, C—H), 2863(w, C—H), 2058′(s, C≡O), 1996 (s, C≡O), 1978 (s, C≡O), 1701 (s, C═O), 1617 (w), 1445 (w), 1427 (w), 1376 (w), 1271 (m, C—O), 1252 (m, C—O), 1177 (m), 1104 (m), 1035 (w), 871 (w), 831 (m), 778 (w), 613 (m);

40. Preparation of (3RS,5RS)-3-[3-(5-bromo-2,4-dioxo-3,4-dihydro-2H-pyrimidin-1-yl)-5-(tert-butyldiphenylsilanylokymethylcyclopent-1enyl]acrylic-acid-ethyl-estertricarbonyliron (rac-44d).

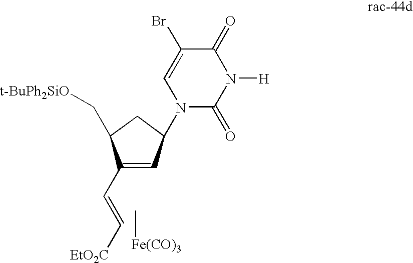

An experiment on a 0.91 mmol scale yielded rac-44d in 49% yield as a yellow-orange oil, starting from rac-43d (according to protocol 36).

¹H NMR (250 MHz, CDCl₃): δ=8.58 (s, 1H, NH), 7.67-7.63 (m, 5H, H-6″+Ph), 7.44-7.35 (m, 6H, Ph), 6.10 (d, J=8.0, 1H, H-3), 5.02 (dd, J₁=9.4, J₂=3.2, 1H, H-3′), 4.12 (m, 2H, OCH₂CH₃), 3.81 (d, J=7.0, 2H,SiOCH₂), 3.14 (m, 1H, H-5′), 2.60 (ddd, J₁=15.7, J₂=J₃=9.1; 1H, H-4ₐ′), 1.76 (s, 1H, H-2′), 1.63 (ddd, J₁=15.9, J₂=J₃=3.6, 1H, H-4_{b}′), 1.26 (t, J=7.1, 3H, OCH₂CH₃), 1.11 (s, 9H, (CH₃)₃C), 0.67 (d, J=7.8, 1H, H-2); FT-R (ATR): 3184 (w, N—H), 3068 (w, N—H), 2928 (w, C—H), 2855 (w, C—H), 2058 (s, C≡O), 1997 (s, C≡O), 1979 (s, C≡O), 1698 (s, C═O), 1617 (w), 1444 (w), 1426 (m), 1375 (w), 1270 (m, C—O); 1238 (m, C—O), 1178 (m), 1111 (m), 1034 (w), 822 (w), 740 (w), 701 (mr), 625 (w)i 613 (m), 609 (m);

41. Preparation of 5-O-(trityl)-D-ribono-1,4-lactone (45′)

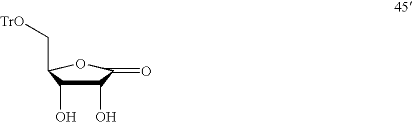

20.1 g of (+)-D-ribonic acid γ-lactone (0.136 mol) was dissolved in 350 ml of pyridine. For the substantial removal of water, about 50 ml of the solvent was distilled off in a rotary evaporator. Then, 41.7 g of chlorotriphenylmethane (0.149 mmol) was added, and a drying tube (CaCl₂) was applied. Stirring was performed at 55° C. over night. The solvent was substantially removed in a rotary evaporator. The residue was transferred into a separation funnel with EtOAc and 0.1% HCl. The organic phase was washed with sat. NaCl solution, and the aqueous phases were re-extracted twice with EtOAc. The combined organic phases were dried over Na₂SO₄, the solvent was removed in a rotary evaporator, and the product was recrystallized from chloroform. The product 45′ crystallized together with one equivalent of chloroform as fine white needles (44.0 g, 63%). Concentrating the mother liquor yielded another 490 mg (4%).

[α]_{D}²⁰=+33.5 (c=1.0 in benzene); ¹H NMR (250 MHz, C₆D₆): δ=2.40 (d, J=1.5 Hz, 1H, HO—C4, exchangeable against D₂O), 2.58 (d, J=4.4 Hz, 1H, HO—C3, exchangeable against D₂O), 2.68 (dd, J=2.6, 10.9 Hz, 1H, CHHO), 3.23 (dd, J=3.2, 10.9 Hz, 1H, CHHO), 3.73 (dd, J=1.5, 5.4 Hz, 1H, H—C4), 4.10 (ψt, 1H, H—C5), 4.63 (dd, J=4.4, 5.4 Hz, 1H, H—C3), 6.95-7.07 and 7.30-7.34 (m, 15H, ArH); ¹³C NMR (62.5 MHz, CDCl₃): δ=63.3 (CH₂O), 69.8 (C-4), 70.6 (C-3), 84.5 (C-5), 88.0 (CAr₃), 127.6, 128.3, 128.4, 143.6 (Ar), 177.2 (C-2); FT-IR: 3416, 3055, 2931, 2872, 1777, 1595, 1488, 1447, 1220, 1181, 1139, 1090, 1031, 1022, 993, 947, 766, 745, 703; MS (EI, 70 eV): 390 (18) [M]⁺, 313 (20), 243 (100), 183 (19), 165 (47); HR-MS (EI, 70 eV): calc. for C₂₄H₂₂O₅: 390.147; found: 390.146; melting point: 104.6° C.

42. Preparation of trifluoromethanesulfonic acid 2-oxo-5-trityloxy-methyl-2,5-dihydrofuran-3-yl ester (46)

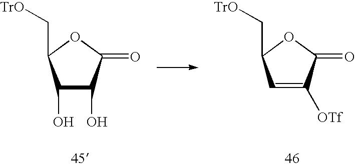

10.45 g of the lactone 45′ (20.5 mmol) was dissolved in 100 ml of dry dichloro-methane and 8.6 ml of dry pyridine (0.107 mol) under a protective gas atmosphere and cooled down to −78° C. A solution of 9.5 ml of trifluoromethanesulfonic anhydride (56.3 mmol) in 35 ml of dry dichloromethane was added dropwise to the mixture. After one hour, the solution was allowed to come to −15° C. and stirred for another hour. After cooling to −78° C., 100 ml of diethyl ether was added, and the solution was filtered through silica gel with another 500 ml of diethyl ether. The solvents were removed in a rotary evaporator, the raw product was purified by column chromatography on silica gel with CyHex/EtOAC (8/1). The product 46 (8.84 g, 85%) was obtained as a white solid, which was recrystallized from hexane/MTBE.

[α]_{D}²⁰=−43.9 (c=1.0 in CHCl₃), ¹H NMR (250 MHz, CDCl₃): δ=3.44 (dd, J_{AB}=10.5 Hz, J=4.3 Hz, 1H, CHHO), 3.55. (dd, J_{AB}=10.5 Hz, J=4.7 Hz, 1H, CHHO), 5.06 (m, J=4.5, 1.9 Hz, 1H, H—C5), 7.11 (d, J=1.9, 1H, H—C4), 7.24-7.40 (m, 15H, ArH); ¹³C NMR (62.5 MHz, CDCl₃): δ=62.7 (CH₂O), 77.7 (C5), 87.4 (Ar₃CO), 118.5 (q, J_{CF}=319 Hz, CF₃), 127.5, 128.1, 128.5 (Ar), 136.1 (C4), 137.8 (C3), 142.8 (Ar), 163.6 (C2); melting point: 140.1° C. (with the color turning to yellow).

43. Preparation of 5-trityloxymethyl-3-vinyl-5H-furan-2-one (47a)

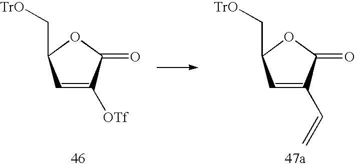

37 mg of Pd₂(dba)₃.CHCl₃ (0.040 mmol) and 86 mg of triphenylarsine (0.28 mmol) were dissolved in 10 ml of dry THF in an argon atmosphere. After the orange-red tetrakis(triphenylarsine)palladium complex had formed, 1.01 g of lithium chloride (24.1 mmol) and 2.55 g of tributylvinylstannane (8.04 mmol) were added. To this mixture was added dropwise a solution of 4.05 g of triflate 46 (8.04 mmol) in 15 ml of abs. THF over a period of 15 min. After 10 min, 2.43 g of cesium fluoride (16 mmol) was added. The mixture was stirred for 2 h, then transferred into a separation funnel with ethyl acetate and washed with 50 ml of half-concentrated sodium chloride solution. After the phases had separated, the organic phase was washed with saturated sodium chloride solution. The aqueous phases were re-extracted twice with ethyl acetate, and the combined organic phases were dried over sodium sulfate. The product was purified by column chromatography on silica gel with cyclohexane/ethyl acetate (12/1). As seen from NMR, the product (2.99 g) still contained 14% tributylchlorostannane (yield of 47a: 87%), but was complexed with nonacarbonyldiiron as described below without further purification. For characterization, an analytical sample of product 47a was recrystallized from cyclohexane/ethyl acetate (15/1).

[α]_{D}²⁰=−41.0 (c=1.0 in CHCl₃); ¹H NMR (250 MHz, CDCl₃): δ=3.35 (dd, J_{AB}=5.3 Hz, J=5.0 Hz, 1H, CH₂O), 4.98 (m, J=5.0, 1.7 Hz, 1H, H—C5), 5.48 (dd, J=2.0, 10.8 Hz, 1H, CHH═CH), 6.27 (dd, J=2.0, 17.7 Hz, 1H, CHH═CH), 6.42 (dd, J=10.8, 17.7 Hz, 1H, CH₂═CH), 7.09 (d, J=1.5 Hz, 1H, H—C4), 7.22-7.32 and 7.38-7.43 (m, 15H, ArH); ¹³C NMR (62.5 MHz, CDCl₃): δ=63.9 (CH₂O), 79.6 (C5), 87.0 (Ar₃CO), 121.6 (CH₂CH), 125.3 (CH₂CH), 127.3 128.0, 128.6 (Ar), 130.5 (C3), 143.3 (Ar), 146.0 (C4), 171.3 (C2); FT-IR 3056, 3020, 2918, 2868, 1963, 1757, 1594, 1488, 1447, 1407, 1345, 1272, 1218, 1152, 1103, 1074, 1031, 1013, 990, 933, 910, 870, 796, 767, 746, 731, 705; MS (EI, 70 eV): 382 (5) [M]⁺, 243 (100), 183 (9), 165 (57), 105 (31), 77(25), 67 (12), 53 (17); HR-MS (EI, 70 eV): calc. for C₂₆H₂₂O₃: 382.157; found: 382.157; melting point: 94.2° C.

44. Preparation of [(1″,2″,3,4-η)-5-trityloxymethyl-3-vinyl-5H-furan-2-one]tricarbonyliron (48a)

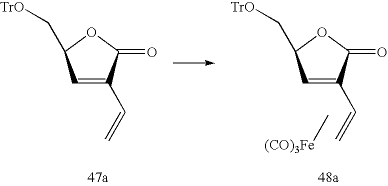

To a solution of 102 mg of 47a (0.267 mmol) in 5 ml of dry THF, 215 mg of Fe₂(CO)₉ (0.590 mmol) was added under an argon atmosphere. The mixture was stirred at room temperature for two hours and then heated under reflux for one hour. Then, 215 mg of Fe₂(CO)₉ was added again, and the mixture was stirred at room temperature for one hour and heated under reflux for another hour. Then, the mixture was filtered through silica gel in air, the solvent was removed in a rotary evaporator, and the residue was purified by column chromatography. Thus, the Fe₃(CO)₁₂ formed as a by-product was first eluted with cyclohexane (CyHex), and then the product was eluted with CyHex/EtOAc (20:1). After removing the solvent in a rotary evaporator, 50 mg of 48a (0.096 mmol, ³⁶%) was obtained as a yellow solid.

¹H NMR (250 MHz, CDCl₃): δ=0.11 (dd, J=2.2, 9.5 Hz, 1H, CHH═CH), 1.64 (dd, J=2.2, 6.8 Hz, 1H, CHH═CH), 2.05 (d, J=0.8 Hz, 1H, H—C4), 3.26 (dd, 4.1, 10.3 Hz, 1H, CHHO), 3.44 (dd, δ=4.1 Hz, 10.3 Hz, 1H, CHHO), 4.30 (ψt, 1H, J=4.1 Hz, 1H, H—C5), 6.24 (dd, J=6.8, 9.5 Hz, 1H, CHH═CH), 7.23 to 4.41 (m, 15H, ArH).

45. Preparation of [(1″,2″,3,4-η)-5-trityloxymethyl-3-vinyl-2,5-dihydrofuran-2-ol]tricarbonyliron (49a)

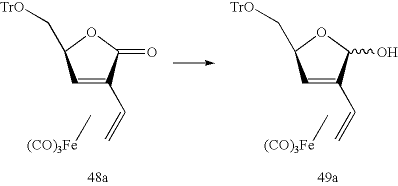

Under an argon atmosphere, 770 μl of 1 M DIBAL-H solution in toluene (0.77 mmol) was added dropwise at −78° C. to a solution of 332 mg of 48a (0.636 mmol) in 20 ml of absolute toluene. The yellow solution was stirred at this temperature for two and a half hours. Then, some drops of acetone were added, the mixture was poured onto 0.5 M tartaric acid solution in a separation funnel and extracted with EtOAc. The organic phases were washed with sat. NaCl solution and dried over Na₂SO₄. Column chromatography on silica gel with CyHex/EtOAc (15:1) yielded the product 49a in the form of a yellow oil as a mixture of diastereomers (NMR) in a yield of 235 mg (0.449 mmol, 71%). This mixture was directly employed for the transformation described below.

46. Preparation of [1′″,2′″,3′,4′-η-(5-methoxy-4-vinyl-2,5-dihydrofuran-2-yl) methanol]tricarbonyliron (50a)

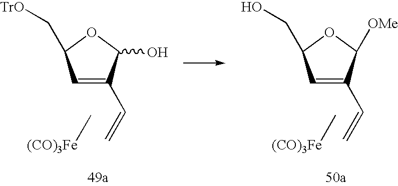

Under an argon atmosphere, 12 mg of p-toluenesulfonic acid monohydrate (0.061 mmol) and 130 μl of o-formic acid trimethyl ester (130 mg, 1.22 mmol) were added to a solution of 90 mg of 49a (0.305 mmol) in 15 ml of absolute MeOH, and the mixture was stirred at room temperature for five hours. Subsequently, two drops of pyridine were added, and the solvent was removed in a rotary evaporator. The residue was taken up in Hex:EtOAc (3.5:1) and filtered through silica gel. After removing the solvent, the product 50a was obtained as a yellow solid (89 mg, 99%).

¹H NMR (250 MHz, CDCl₃): δ=−0.01 (dd, J=2.2, 8.8 Hz, 1H, CHH═CH), 1.68 (dd, J=2.2, 6.6 Hz, 1H, CHH═CH), 1.73 (s, 1H, H—C4′), 2.83 (dd, J 2.5, 10.2 Hz, 1H, OH), 3.36 to 3.59 (m, 4H, CHHO and CH₃O), 3.75 (ψt, 2.6, 12.1 Hz, 1H, CHHO), 4.33 (ψt, 1H, J=2.9, 8.1 Hz, 1H, H—C5′), 5.53 (s, 1H, H—C 2′), 5.59 (dd, J=6.6, 8.8 Hz, 1H, CHH═CH).

47. Preparation of [1′″,2′″,3,4-η-5-(dimethyl-(1,1,2-trimethylpropyl)-silanyloxymethyl)-2-methoxy-3-vinyl-2,5-dihydrofurain]tricarbonyliron (51a)

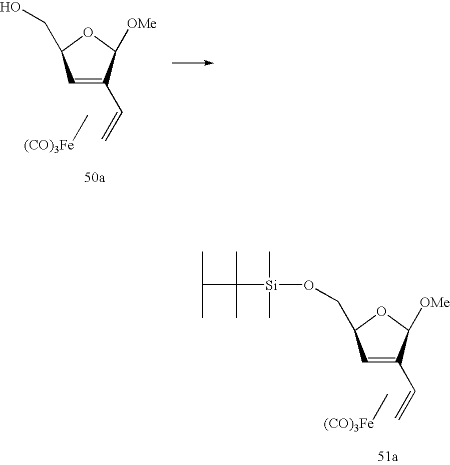

Under an argon atmosphere, 90 μl of thexyldimethylsilyl chloride (80 mg, 0.44 mmol) was added dropwise to a solution of 87 mg of 50a (0.294 mmol) and 100 mg of imidazole (1.47 mmol) in 3 ml of absolute dichloromethane. The yellow solution was stirred at room temperature over night. Then, the solution was diluted with dichloromethane, water was added, and stirring was continued for another hour. After the phases had separated, the aqueous phase was re-extracted twice with dichloromethane. After drying the combined organic phases over Na₂SO₄, the product 51a was obtained by chromatography on silica gel with CyHex:EtOAc (15:1) with a yield of 124 mg (0.283 mmol, 96%) as a yellow oil.

¹H NMR (250 MHz, CDCl₃): δ=−0.08 (dd, J=2.2, 8.8 Hz, 1H, CHH═CH), 0.08 and 0.09 (each s, 6H, Si(CH₃)₂), 0.81 (s, 6H, SiC(CH₃)₂), 0.82 (d, J=6.8 Hz, 6H, CH(CH₃)₂), 1.57 (sept, J=6.8 Hz, 1H, CH(CH₃)₂), 1.64 (dd, J=2.2, 6.6 Hz, 1H, CHH═CH), 1.99 (s, 1H, H—C4), 3.44 (dd, J=8.1 Hz, 9.7 Hz, 1H, CHHO), 3.74 (dd, 5.9, 9.7 Hz, 1H, CHHO), 4.09 (dd, 1H, J=5.9, 8.1 Hz, 1H, H—C5), 5.52 (s, 1H, H—C2), 5.59 (dd, J=6.6, 8.8 Hz, 1H, CHH═CH).

48. Preparation of [4-amino-1-{η⁴-5-[dimethyl-(1,1,2-trimethylpropyl)-silanyloxymethyl]-3-vinyl-2,5-dihydrofuran-2-yl}-3,4-dihydro-1H-pyrimidin-2-one]tricarbonyliron (52a)

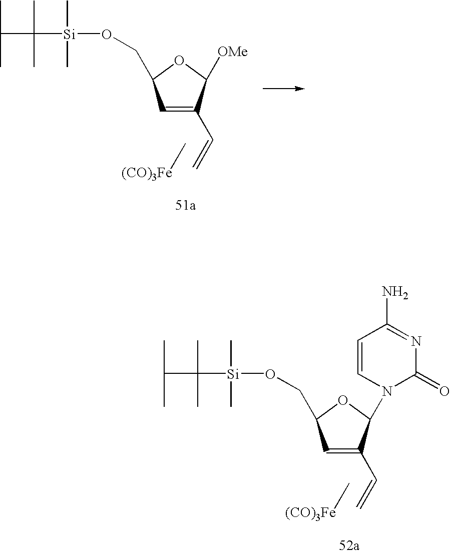

Under an argon atmosphere, 290 μl of trimethylsilyl triflate (356 mg, 1.60 mmol) was added dropwise to a solution of 117 mg of 51a (0.267 mmol) and 275 mg of 2,4-bis(trimethylsiloxy)cytosine (1.07 mmol) in 6 ml of dry dichloromethane. The yellow solution was stirred at room temperature for four hours and then added dropwise to 20 ml of sat. NaHCO₃ solution. After the phases had separated, the aqueous phase was extracted twice with dichloromethane, the combined organic phases were washed with sat. NaCl solution and dried over Na₂SO₄. The product (52a) was obtained by column chromatography on silica gel with EtOAc/MeOH (30:1) in a yield of 116 mg (0.224 mmol, 84%) as a light yellow solid.

¹H NMR (250 MHz, CDCl₃): δ=−0.16 (dd, J=2.2, 8.9 Hz, 1H,CHH═CH), 0.08 and 0.09 (each s, 6H, Si(CH₃)₂), 0.81 (s, 6H, SiC(CH₃)₂), 0.82 (d, J=6.8 Hz, 6H, CH(CH₃)₂), 1.57 (sept, J=6.8 Hz, 1H, CH(CH₃)₂), 1.68 (dd, J=2.2, 6.7 Hz, 1H, CHH═CH), 1.85 (s, 1H, H—C4′), 3.72 (dd, J_{AB}=14.1 Hz, J=4.5 Hz, 1H, CHHO), 3.78 (dd, J_{AB} 14.1 Hz, J=4.1 Hz, 1H, CHHO), 4.30 (ψt, 1H, H—C5′), 5.73 (d, J=7.4 Hz, 1H, HC5), 6.06 (ψt, 1H, CHH═H), 7.00 (s, 1H, H—C2′), 7.98 (d, J=7.4 Hz, 1H, H—C6); FT-IR 3343, 3104, 2955, 2046, 1968, 1660, 1632, 1484, 1400, 1276, 1250, 1170, 1108, 1079, 1033, 830, 775, 730; MS (ESI, 70 eV): 540 (100) [M—Na]⁺, 518 (41) [M—H]⁺, 407 (76), 276 (39); HR-MS (ESI; 70 eV): calc. for C₂₂H₃₁FeN₃O₆Si—H: 518.141; found: 518.140.

### References

Y. A. Hannun (1997), Apoptosis and the dilemma of cancer therapy, Blood 89, 1845-1853.

J. A. Hickman (1996), Apoptosis and chemotherapy resistance, Eur. J. Cancer 32A, 921-926.

M. Raisova, M. Bektas, T. Wieder, P. T. Daniel, J. Ebene, C. E. Orfanos, C. C. Geilen (2000), Resistance to CD95/Fas-induced and ceramide-mediated apoptosis of human melanoma cells is caused by a defective mitochondrial cytochrome c release, FEBS Lett. 473, 27-32.

G. M. Cohen (1997), Caspases: the executioners of apoptosis, Biochem. J. 326, 1-16.

F. Eβmann, T. Wieder, A. Otto, E.-C. Müller, B. Dörken, P. T. Daniel (2000), The GDP dissociation inhibitor, D4-GDI (Rho-GDI 2), but not the homologous Rho-GDI 1, is cleaved by caspase-3 during drug-induced apoptosis, Biochem. J. 346, 777-783.

E. Heβler, H.-G. Schmalz, G. Dürner (1994),° C.hiral Butadiene-Fe(CO)₃ Complexes for Organic Synthesis. Reactions of (η⁴-2-Alkoxy-4-vinyl-2,5-dihydrofuran)-Fe(CO)₃ Derivatives, Tetrahedron Lett. 35, 4547-4550.

N. Rehnberg, G. Magnusson (1990), Chiral Aldehydes by Ring Contraction of Pento- and Hexapyranoside Epoxides, J. Org. Chem. 55, 5467-5476.

T. Wieder, C. Perlitz, M. Wieprecht, R. T. C. Huang, C. C. Geilen, C. E. Orfanos (1995), Two new sphingomyelin analogues inhibit phosphatidylcholine biosynthesis by decreasing membranebound CTP: phosphocholine cytidylyltransferase levels in HaCaT cells, Biochem. J. 311, 873-879.

T. Wieder, F. Eβmann, A. Prokop, K. Schmeiz, K. Schulze-Osthoff, R. Beyaert, B. Dörken, P. T. Daniel (2001), Activation of caspase-8 in drug-induced apoptosis of B-lymphoid cells is independent of CD95/Fas receptor-ligand interaction and occurs downstream of caspase-3, Blood 97, 1378-1387.

Figures:

FIG. 1:

Pro-apoptotic effect of the nucleoside analogues N76 and N69 in malignant lymphoblastic cells. The preparation of N69 contained impurities in the form of an isomer.

FIG. 2:

N76-induced activation of caspase-3 in malignant lymphoblastic cells.

FIG. 3:

Pro-apoptotic effect of the nucleoside analogue N69 in primary cells of patients suffering from various malignant diseases of the hematopoetic system. The N69 preparation contained impurities in the form of an isomer.

FIG. 4:

Pro-apoptotic effect of the nucleoside analogues N69 and N76 in primary cells of patients as compared with established cytostatic agents.

FIG. 5:

Influence of the complexes iron on the antiproliferative and cell-death inducing effects of N69.

FIG. 6:

The carbocyclic nucleoside analogue JV-206-1 (structural formula 35) induces cell death in BJAB cells via the mitochondrial apoptosis signal pathway.

1. A medicament containing at least one compound of structural formulas 1a or 1b: 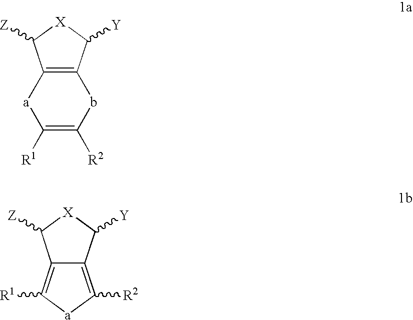
wherein
X=O, S, CH₂, NR, no bond, with the proviso that, if X=O, then the compound of formula 1a is associated with a metal-ligand complex according to formula 1′a;
Y=adenine, cytosine, guanine, uracil, thymine, bromouracil, purines or pyrimidines and their derivatives, nucleobases, heterocycles, aminoalkyl, aminoaryl or other residues which are capable of hydrogen bonding;
Z=alkyl, fluoroalkyl, aryl, fluoroaryl, —(CH₂)ₙOR′, with R′=H, SiR₃, alkyl, fluoroalkyl, aryl, fluoroaryl, acyl, and n=0 to 5, wherein preferably n=1;
a=no bond, a single bond or CH₂, in formula 1b also CH₂CH₂;
b=no bond, a single bond or CH₂; a and b being selected in such a way that the moiety comprising a and b includes a 1,3-diene;
R¹=—H, F, alkyl, fluoroalkyl, aryl, fluoroaryl, OR, —CO₂R, —SO₂OR, —CONR₂;
R²=—H, F, alkyl, fluoroalkyl, aryl, fluoroaryl, OR, —CO₂R, —SO₂OR, —CONR₂;
R=H, branched and linear alkyls, especially methyl, ethyl, propyl, thexyl, tert-butyl;
and/or salts thereof.

2. The medicament according to claim 1, characterized by having the structural formula 1′a or 1′b: 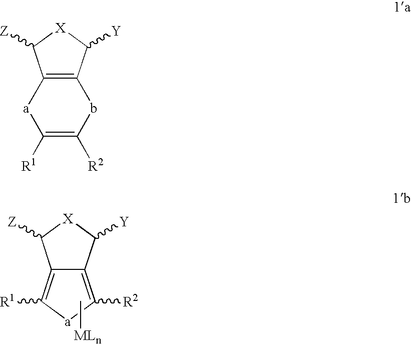
wherein:
n is an integer of from 2 to 4;
M=Mn, Fe, Co, Ru; and
L=CO, CN—R, CN, CR₂, COR, Hal, cyclopentadienyl (Cp) or a substituted Cp derivative; and
the same or different Ls may be bonded to one M.

3. The medicament according to claim 2, characterized in that said at least one compound of structural formula 1′a or 1′b has the structural formula 2, 3, 4a, 4b or 4c: 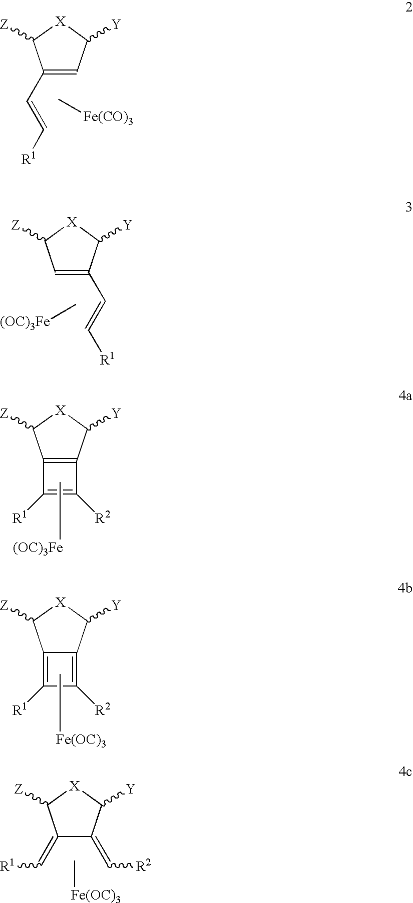
wherein
the Fe(CO)₃ unit is η⁴-bonded;
X=O, CH₂ or no bond; and
Y, Z, R¹ and R² are selected as in claim 1.

4. The medicament according to claim 3, characterized in that said at least one compound of structural formula 2 has the structural formula 5 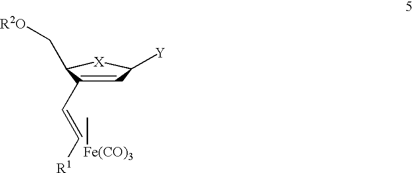
wherein
X=O, CH₂;
Y=adenine, cytosine, guanine, uracil, thymine, bromouracil, purines or pyrimidines and their derivatives, nucleobases, heterocycles;
R¹=—H, —CO₂R;
R²=H, SiR₃, alkyl, fluoroalkyl, aryl, fluoroaryl, acyl;
wherein R is selected as in claim 1.

5. Compounds of structural formula 5 as defined in claim 4, characterized in that:
R²=SiR₃, X=O;
Y and R¹ are selected as in claim 4, and R is selected as in claim 1, with the proviso that, if Y is bromouracil, uracil or methyluracil, then R² is not thexyl(CH₃)₂Si.

6. Compounds according to claim 5, selected from the group consisting of:

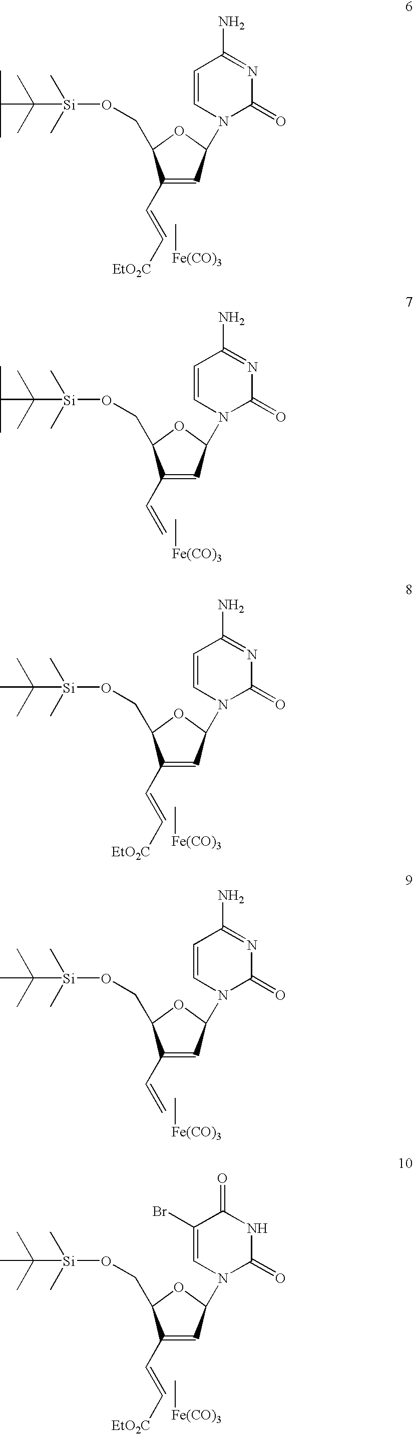

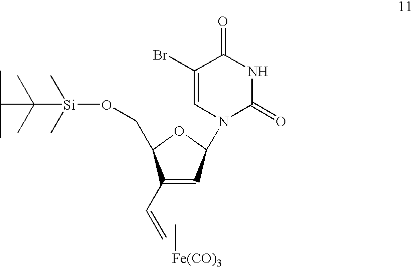

7. The compound of structural formula 5 as defined in claim 4, selected from the group consisting of:

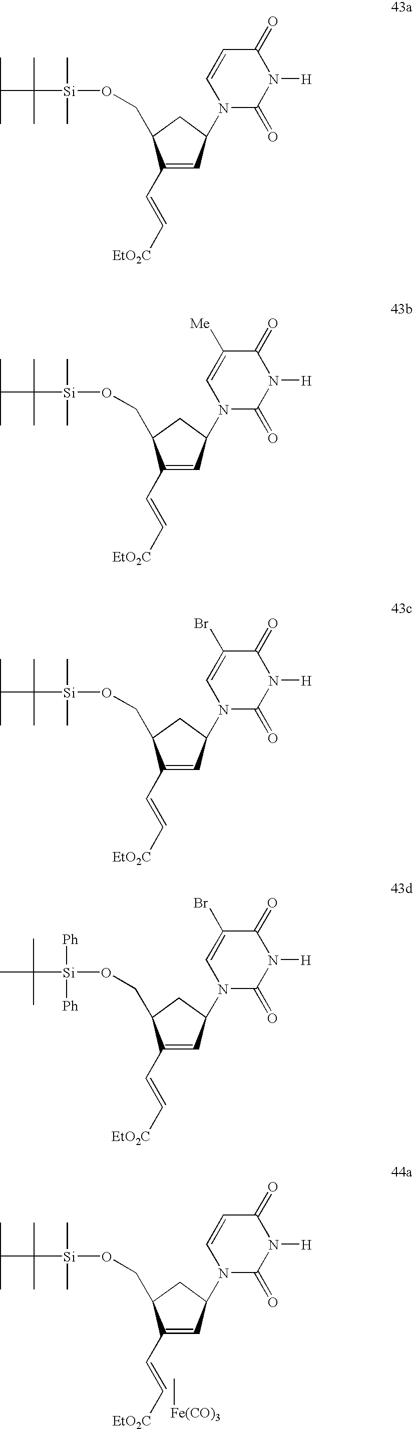

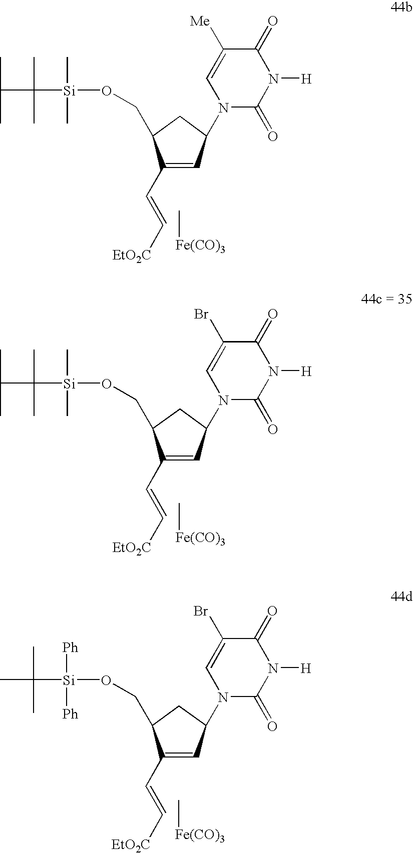

8. Use of compounds according to any of claims 1 to 7 and/or of compounds of structural formula 1a: 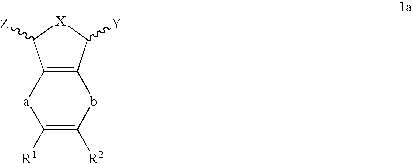
wherein
X=O, S, CH₂, NR or no bond; and
Y, Z, ar b, R¹ and R² are selected as in claim 1;
for preparing a medicament for the treatment of malignant diseases of the bone marrow or other hematopoetic organs, solid tumors, epithelial tumors, benign or semimalignant fast-proliferating tumors or skin diseases, especially psoriasis vulgaris, keloids and basaliomas, lymphomas, especially Hodgkin's and non-Hodgkin lymphomas, inflammatory, chronic inflammatory, bacterial and auto-immune diseases, and for antibacterial, antimycotic, antiprotozoan, antiplasmodium, antihelminthic or immunosuppressant therapies and/or for inducing apoptosis.

9. A process for the synthesis of compounds according to claim 5, 6 and/or 7, characterized by comprising the reaction step of a diastereoselective iron-supported nucleophilic substitution, preferably using silylated nucleobases in the presence of a Lewis acid, and that, in this reaction step, a starting substance which has an etherified or esterified hydroxy group at the Y position localized as in structural formula 5 is reacted to introduce a nucleobase in place of this hydroxy group.
